(19) United States
(12) Patent Application Publication
(10) Pub. No.: US 2023/0338363 A1
(43) Pub. Date: Oct. 26, 2023

(54) COMBINATION CHEMO-IMMUNOTHERAPY FOR PANCREATIC CANCER USING THE IMMUNOGENIC EFFECTS OF AN IRINOTECAN SILICASOME NANOCARRIER PLUS ANTI-PD-1

(71) Applicant: The Regents of the University of California, Oakland, CA (US)

(72) Inventors: Andre E. Nel, Sherman Oaks, CA (US); Huan Meng, Los Angeles, CA (US); Xiangsheng Liu, Los Angeles, CA (US)

(73) Assignee: The Regents of the University of California, Oakland, CA (US)

(21) Appl. No.: 18/019,459

(22) PCT Filed: Aug. 2, 2021
(86) PCT No.: PCT/US2021/044192

§ 371 Date: Feb. 2, 2023

Related U.S. Application Data
(60) Provisional application No. 62/706,308, filed on Aug. 7, 2020.

(51) Int. Cl. A61K 31/4745; C07K 16/28; A61K 31/4706; A61K 33/244; A61K 45/06; A61P 35/00

(52) U.S. Cl. CPC A61K 31/4745; A61K 31/4706; A61K 33/244; A61K 45/06; A61P 35/00; C07K 16/2818; C07K 16/2827; A61K 9/0019

(57) ABSTRACT

In various embodiments, methods of treating a cancer are provided. In certain embodiments, the methods comprise administering to a mammal in need thereof i) one or more checkpoint inhibitor(s); and ii) one or more camptothecin analogs, and, optionally, or one or more autophagy inhibitors wherein said camptothecin analog, and one or more autophagy inhibitors, when present, are provided inside a delivery vehicle where said delivery vehicle comprises: a nanoparticle comprising one or more cavities disposed within said nanoparticle and an outside surface where said one or more cavities are in fluid communication the outside surface of said nanoparticle; said one or more camptothecin analog, one or more autophagy inhibitors, when present, are disposed within said one or more cavities; and a lipid bilayer is disposed on the surface of said nanoparticle where said lipid bilayer fully encapsulates the nanoparticle.

### CROSS-REFERENCE TO RELATED APPLICATIONS

This application claims benefit of and priority to USSN 62/706,308, filed on Aug. 7, 2020, which is incorporated herein by reference in its entirety for all purposes.

### STATEMENT OF GOVERNMENTAL SUPPORT

This invention was made with government support under Grant Number CA198846, awarded by the National Institutes of Health. The government has certain rights in the invention.

### INCORPORATION BY REFERENCE OF SEQUENCE LISTING PROVIDED AS A TEXT FILE

[ Not Applicable ]

### BACKGROUND

Pancreatic ductal adenocarcinoma (PDAC) is a lethal disease with a 5-year survival rate of ~8%^{[1]}. According the guidelines of the American Cancer Society, the best available chemotherapy options for advanced disease are treatment with gemcitabine (GEM)/Nab-paclitaxel or a four-drug regimen, known as FOLFIRINOX (folinic acid, 5-fluorouracil, irinotecan, oxaliplatin) (FIG. 1, panel A)^{[2]}. The FOLFIRINOX regimen was modified in 2018 to allow resected PDAC patients to be treated with a reduced irinotecan dose (150 instead of 180 mg/m²) for 24 weeks^{[2d]}. The outcome was encouraging in the patients who had been resected, demonstrating a median overall survival was 54.4 months in the modified FOLFIRINOX group vs 35.0 months in the control arm (GEM monotherapy) (p < 0.01)^{[2a,} ^{2d]}. While more clinical validation studies are ongoing, it is suggested that the modified regimen is gaining support. Moreover, recent studies of liposomal irinotecan (ONIVYDE®) have led to its approval in metastatic pancreas cancer in patients having progressed on gemcitabine^{[3]}. This provided the first evidence that a liposomal formulation of irinotecan in pancreatic cancer has clinical utility.

In addition to new ways in which chemotherapy is being used, we are drawing on the game-changing advances that have been introduced immune checkpoint inhibitors (ICIs) to treat cancers such as melanoma, renal, and lung cancer^{[4]}. However, there has been little success in the use of immune checkpoint blocking antibodies in PDAC^{[5]}. While anti-PD-1 antibody (Keytruda®) was approved for PDAC patients with rare genetic mutations (i.e. microsatellite instability or mismatch repair deficiency), this treatment option impacts less than 1% of cases^{[6]}. Although the exact rate of PD-L1 expression in PDAC is controversial, several studies have suggested that this biomarker is expressed in only ~10% of cases^{[7]}. However, higher rates have also been reported ^{[8]}, which is indicative of the heterogeneous PDAC immune landscape as well as the lack of consensus in how to perform PD-L1 quantification^{[9]}. Nevertheless, it is generally agreed upon that the general lack of expression of immune checkpoint receptors is a key reason for the poor response to PD-1/PD-L1 blockade in this disease ^{[7a,} ^{9]}. Other factors, such as low tumor immunogenicity (“cold tumors”), low mutational load, poor drug access, accumulation of regulatory T-cells (Tregs), stroma-mediated immunosuppression, and regional expression of a host of additional immune escape pathways are also contributory to failed immunotherapy in PDAC^{[10]}.

In spite of the poor response to chemotherapy, it has become popular for many solid tumors, including PDAC, to address the immune-suppressive tumor microenvironment (TME) by introducing combination therapy in an attempt to augment the ICI responsiveness ^{[5a,} ^{11]}. A promising approach is to utilize the immunogenic properties of select chemotherapeutic agents, such as anthracyclines (e.g. doxorubicin, DOX) or oxaliplatin (OX), capable of increasing the recruitment of cytotoxic T cells (CTL) to the “cold” TME, i.e., switching its immune status to “hot”^{[12]}. This immunogenic effect is dependent on off-target effects of the chemo agents on cellular sites such as the endoplasmic reticulum (ER), where the generation of cell stress responses can trigger translocation of calreticulin (CRT) to the dying tumor cell surface; CRT serves as an “eat me” signal for cancer cell engulfment by antigen-presenting cells (APC) ^{[12e, 12f, 13]}. This allows APC display of endogenous tumor-associated antigens to naïve T-cells^{[12e, 12f, 13]}. In addition, disintegration of the nuclei of dying tumor cells, leads to the release of high-mobility group box 1 (HMGB 1) protein, which acts as an adjuvant by engaging TLR4 receptors on APC ^{[12e, 12f, 13]}. There is also a critical contribution to the immunogenic effects of above chemo agents through induction of autophagy and ATP release^{[12f, 14]}. The collective effect of CRT, HMGB1 and autophagy is to generate immunogenic cell death (ICD) responses by above chemo agents to provide endogenous vaccination effect that can be used to complement the chemotherapy response. Moreover, ICD leads to the activation and recruitment of cytotoxic T-cell-lymphocytes (CTL), the killing effect of which can be boosted by the use of checkpoint blocking antibodies^{[12c-e, 12g]}. In PDAC, for example, it has been demonstrated that oxaliplatin is capable of triggering immunogenic effects in human PANC-1 and murine Pan02 models ^{[12b]}. However, the deliberate implementation of chemotherapeutic agents to induce immune responses has not as yet been accomplished as a reproducible treatment option in the clinic because it is difficult to control the delivery of ICD stimuli, which is a particular challenge for PDAC in light of the restricted drug access to the tumor site as a result of the dysplastic stroma^{[15].}

### SUMMARY

There is an urgent need to develop new life-prolonging therapy for pancreatic ductal adenocarcinoma (PDAC). As described herein, we demonstrate that improved irinotecan delivery by a lipid bilayer coated mesoporous silica nanoparticle (MSNP), a.k.a. silicasome, can improve PDAC survival through the generation of a chemo-immunotherapy response in an orthotopic Kras-dependent pancreatic cancer (KPC) model. This discovery is premised, in part, on the weak-basic properties of irinotecan, which neutralizes the acidic lysosomal pH in PDAC cells. This effect triggers a linked downstream cascade of events that include autophagy inhibition, endoplasmic reticulum stress, immunogenic cell death (ICD) and PD-L1 expression. ICD was characterized by calreticulin expression and HMGB1 release in dying KPC cells, which was demonstrated in a vaccination experiment to prevent KPC tumor growth on the contralateral site. The improved delivery of irinotecan by silicasome at the tumor site was accompanied by robust anti-tumor immunity, which could be synergistically enhanced by anti-PD-1 antibody. Immunohistochemistry confirmed the expression of CRT, HMGB1, PD-L1 and an autophagy marker, LC3B, at the tumor site, in addition to the triggering of perforin and granzyme B release, and elevation of the CD8⁺/FoxP3⁺ cells ratio. Moreover, the chemo-immunotherapy response elicited by the silicasome was more robust than the response to free drug or an irinotecan-delivery liposome (ONIVYDE®). Animal survival was significantly enhanced by combination therapy with the silicasome plus anti-PD-1 and was far superior to the combination immunotherapy response to either free irinotecan or ONIVYDE®.

Accordingly, various embodiments contemplated herein may include, but need not be limited to, one or more of the following:

Embodiment 1: A method of treating a cancer in a mammal, said method comprising:

administering to said mammal, or causing to be administered to said mammal, an effective amount of:
i) one or more checkpoint inhibitor(s); and
ii) one or more camptothecin analogs, and, optionally, or one or more autophagy inhibitors wherein said camptothecin analog, and one or more autophagy inhibitors, when present, are provided inside a delivery vehicle where said delivery vehicle comprises:
   a nanoparticle comprising one or more cavities disposed within said nanoparticle and an outside surface where said one or more cavities are in fluid communication the outside surface of said nanoparticle;
   said one or more camptothecin analogs, and said one or more autophagy inhibitors, when present, are disposed within said one or more cavities; and
   a lipid bilayer is disposed on the surface of said nanoparticle where said lipid bilayer fully encapsulates the nanoparticle.

Embodiment 2: The method of embodiment 1, wherein said one or more checkpoint inhibitors and said one or more camptothecin analogs are administered to said subject simultaneously.

Embodiment 3: The method of embodiment 2, wherein said one or more checkpoint inhibitors and said one or more camptothecin analogs are administered in a combined formulation.

Embodiment 4. The method of embodiment 1, wherein said one or more checkpoint inhibitors and said one or more camptothecin analogs are administered at different times.

Embodiment 5: The method according to any one of embodiments 1-4, wherein said camptothecin analog comprises irinotecan.

Embodiment 6: The method according to any one of embodiments 1-5, wherein said camptothecin analog comprises a camptothecin analog other than irinotecan.

Embodiment 7: The method of embodiment 6, wherein said camptothecin analog comprises an analog selected from the group consisting of belotecan (CKD-602), topotecan, silatecan (db-67, ar-67), cositecan (bnp-1350), exatecan, lurtotecan, gimatecan (st1481), rubitecan, homocamptothecin, trastuzumab deruxtecan, Rubitecan, Beltecan, Exatecan, Lurtotecan, Gimatecan, Diflomotecan, Karenitecan, Silatecan, Namitecan, ZBH-1205, Elomotecan, DRF-1042, Delimotecan, NSC606985, Chimmitecan, Genz-644282, and non-CPT1.

Embodiment 8: The method according to any one of embodiments 6-7, wherein said camptothecin analog comprises a weakly basic analog.

Embodiment 9: The method according to any one of embodiments 6-7, wherein said camptothecin analog comprises a water soluble analog.

Embodiment 10: The method of embodiment 9, wherein said water soluble analog has a solubility of greater than 5 mg/mL in water, or greater than 8 mg/mL in water, or greater than 10 mg/mL in water, or greater than about 12 mg/mL in water or greater than about 15 mg/mL in water, or greater than about 20 mg/mL in water, or greater than about 22 mg/mL in water.

Embodiment 11: The method according to any one of embodiments 7-10, wherein said camptothecin analog comprises belotecan (CKD-602).

Embodiment 12: The method according to any one of embodiments 1-11, wherein said checkpoint inhibitor comprises one or more checkpoint inhibitors selected from the group consisting of a PD-L1 inhibitor, a PD-1 inhibitor, and a CTLA-4 inhibitor.

Embodiment 13: The method of embodiment 12, wherein said checkpoint inhibitor comprises one or more PD-L1 inhibitors.

Embodiment 14: The method of embodiment 13, wherein said checkpoint inhibitor comprises an anti-PD-L1 antibody.

Embodiment 15: The method of embodiment 14, wherein said checkpoint inhibitor comprises an anti-PD-L1 antibody selected from the group consisting of Atezolizumab, Avelumab, Durvalumab, BMS-936559, RG-7446. MPDL3280A, MEDI-4736, and MSB0010718C.

Embodiment 16: The method of embodiment 13, wherein said checkpoint inhibitor comprises a peptidic PD-L1 inhibitor.

Embodiment 17: The method of embodiment 16, wherein said PD-L1 inhibitor comprise a moiety selected from the group consisting of AUNP12, CA-170, and BMS-986189.

Embodiment 18: The method according to any one of embodiments 12-17, wherein said checkpoint inhibitor comprises a PD1 inhibitor.

Embodiment 19: The method of embodiment 18, wherein said checkpoint inhibitor comprises an anti-PD1 antibody.

Embodiment 20: The method of embodiment 19, wherein said checkpoint inhibitor comprises an anti-PD1 antibody selected from the group consisting of Nivolumab, Pembrolizumab, Cemiplimab, avelumab, durvalumab, and atezolizumab.

Embodiment 21: The method of embodiment 18, wherein said checkpoint inhibitor comprises an fc fusion with PD-L2.

Embodiment 22: The method of embodiment 21, wherein said checkpoint inhibitor comprises AMP224.

Embodiment 23: The method according to any one of embodiments 12-22, wherein said checkpoint inhibitor comprises CTLA-4 inhibitor.

Embodiment 24: The method of embodiment 23, wherein said CTLA-4 inhibitor comprises Ipilimumab.

Embodiment 25: The method according to any one of embodiments 1-11, wherein said checkpoint inhibitor comprises a bispecific antibody that binds to two checkpoint inhibitors, or an antibody that binds to a checkpoint inhibitor attached to a cytokine.

Embodiment 26: The method of embodiment 25, wherein said checkpoint inhibitor comprises a bispecific antibody that binds to two checkpoint inhibitors.

Embodiment 27: The method of embodiment 26, wherein said bispecific antibody comprises an antibody that binds to PD-1 attached to an antibody that binds to PD-L1, or an antibody that binds to PD-1 attached to an antibody that binds to CTLA4, or an antibody that binds to PD-L1 attached to an antibody that binds to CTLA4.

Embodiment 28: The method of embodiment 27, wherein said bispecific antibody comprises an antibody that binds to PD-1 attached to an antibody that binds to CTLA4.

Embodiment 29: The method of embodiment 25, wherein said checkpoint inhibitor comprises a cytokine attached to an antibody that binds to a checkpoint inhibitor.

Embodiment 30: The method of embodiment 29, wherein said checkpoint inhibitor comprises a cytokine attached to an antibody selected from the group consisting of anti-PD-1, anti-PD-L1, and CTLA4.

Embodiment 31: The method of embodiment 30, wherein said checkpoint inhibitor comprises cytokine attached to an anti-PD-1 antibody.

Embodiment 32: The method of embodiment 31, wherein said checkpoint inhibitor comprises an IL-7 attached to an anti-PD-1 antibody.

Embodiment 33: The method according to any one of embodiments 1-32, wherein said drug delivery vehicle contains one or more autophagy inhibitors.

Embodiment 34: The method of embodiment 33, wherein said one or more autophagy inhibitors comprises an agent selected from the group consisting of chloroquine, hydroxychloroquine, and a member of the bafilomycin family.

Embodiment 35: The method of embodiment 34, wherein said one or more autophagy inhibitors comprises chloroquine.

Embodiment 36: The method according to any one of embodiments 34-35, wherein said one or more autophagy inhibitors comprises hydroxychloroquine.

Embodiment 37: The method according to any one of embodiments 34-36, wherein said one or more autophagy inhibitors comprises a member of the bafilomycin family.

Embodiment 38: The method of embodiment 37 wherein said one or more autophagy inhibitors comprises a member of the bafilomycin family selected from the group consisting of bafilomycin A1, bafilomycin B1, bafilomycin B2, bafilomycin C1, bafilomycin C2, bafilomycin C1 amide, bafilomycin C2 amide, 9-hydroxybafilomycin D, 29-hydroxybafilomycin D, bafilomycin D, and bafilomycin E.

Embodiment 39: The method of embodiment 33, wherein said one or more autophagy inhibitors comprises one or more autophagy inhibitors shown in Table .

Embodiment 40: The method according to any one of embodiments 33-39, wherein said one or more autophagy inhibitors comprises said autophagy inhibitor comprises an autophagy-inhibiting nanoparticle (e.g., disposed inside the nanoparticle comprising said drug delivery vehicle).

Embodiment 41: The method of embodiment 40, wherein said autophagy-inhibiting nanoparticle comprises a metal or metal oxide, a rare earth or rare earth oxide, or silica.

Embodiment 42: The method of embodiment 41, wherein said autophagy-inhibiting nanoparticle comprises a metal or metal oxide.

Embodiment 43: The method of embodiment 41, wherein said autophagy-inhibiting nanoparticle comprises a metal.

Embodiment 44: The method of embodiment 43, wherein said autophagy-inhibiting nanoparticle comprise a metal selected from the group consisting of gold, silver, iron, copper, and titanium.

Embodiment 45: The method of embodiment 41, wherein said autophagy-inhibiting nanoparticle comprises a metal oxide.

Embodiment 46: The method of embodiment 45, wherein said autophagy-inhibiting nanoparticle comprises a metal oxide selected from the group consisting of zinc oxide, iron oxide, iron oxide/gold, copper oxide, titanium dioxide, and ferroferic oxide.

Embodiment 47: The method of embodiment 41, wherein said autophagy-inhibiting nanoparticle comprises a rare earth or rare earth oxide.

Embodiment 48: The method of embodiment 47, wherein said autophagy-inhibiting nanoparticle comprise a rare earth selected from the group consisting of scandium, yttrium, lanthanum, cerium, praseodymium, neodymium, promethium, samarium, europium, gadolinium, terbium, dysprosium, holmium, erbium, thulium, ytterbium, and lutetium.

Embodiment 49: The method of embodiment 47, wherein said autophagy-inhibiting nanoparticle comprise a rare earth oxide selected from the group consisting of cerium oxide, and europium hydroxide.

Embodiment 50: The method according to any one of embodiments 1-49,wherein said nanoparticle comprise a single cavity.

Embodiment 51: The method of embodiment 50, wherein said nanoparticle comprises a nanobowl.

Embodiment 52: The method according to any one of embodiments 1-49, wherein said nanoparticle comprises a plurality of cavities.

Embodiment 53: The method of embodiment 52, wherein said nanoparticle comprises a porous inorganic nanoparticle, a metal-organic framework nanoparticle, or a porous organic nanoparticle.

Embodiment 54: The method of embodiment 53, wherein said nanoparticle comprise a porous inorganic nanoparticle.

Embodiment 55: The method of embodiment 54, wherein said nanoparticle comprise a porous silica nanoparticle, a porous calcium carbonate nanoparticle, or a porous calcium phosphate nanoparticle.

Embodiment 56: The method of embodiment 55, wherein said nanoparticle comprises a porous silica nanoparticle.

Embodiment 57: The method of embodiment 56, wherein said nanoparticle comprises a mesoporous silica nanoparticle (MSN), a mesoporous organosilica nanoparticle (MONs), or a periodic mesoporous organosilica (PMO) nanoparticle.

Embodiment 58: The method of embodiment 57, wherein said nanoparticle comprises a mesoporous silica nanoparticle (MSN).

Embodiment 59: The method of embodiment 58, wherein said nanoparticle comprises undoped and unfunctionalized silica.

Embodiment 60: The method according to any one of embodiments 57-58, wherein said nanoparticle comprises a mesoporous silica /hydroxyapatite (MSNs/HAP) hybrid nanoparticle.

Embodiment 61: The method according to any one of embodiments 57-58, wherein said nanoparticle comprises a cleavable silsesquioxane, or a bridged silsesquioxane (BS).

Embodiment 62: The method according to any one of embodiments 57-58, wherein said nanoparticle comprises an inorganically doped silica.

Embodiment 63: The method of embodiment 62, wherein said nanoparticle comprises a calcium-, iron-, manganese-, or zirconium-doped silica.

Embodiment 64: The method according to any one of embodiments 57-58, wherein said nanoparticle comprises an imine-doped silica.

Embodiment 65: The method of embodiment 55, wherein said nanoparticle comprises a mesoporous calcium carbonate nanoparticle.

Embodiment 66: The method of embodiment 55, wherein said nanoparticle comprises a mesoporous calcium phosphate nanoparticle.

Embodiment 67: The method of embodiment 53, wherein said nanoparticle comprises a porous biocompatible polymer.

Embodiment 68: The method of embodiment 67, wherein said nanoparticle comprise a porous biocompatible polymer selected from the group consisting of polymers of the polyaryletherketone (PAEK) family (e.g., polyether ether ketone (PEEK), carbon reinforced PEEK, polyether ketone ketone (PEKK), PEKEKK (polyetherketoneetherketoneketone), polyaryletherketone (PAEK), polyetherketone (PEK), Polyetherketone Etherketone Ketone (PEKEKK), and the like), polycaprolactone (PCL), polylactic acid (PLA), polyglycolic acid (PGA), polyphenylene, self-reinforced polyphenylene, polyphenylsulphone, polysulphone, polyethylene terephthalate (PET), polyethylene, polyurethane, oligocarbonatedimethacrylate (OCM-2) porous polymer, carbonate- and phthalate-containing dimethacrylates, and the like.

Embodiment 69: The method of embodiment 67, wherein said nanoparticle comprises a hydrogel.

Embodiment 70: The method of embodiment 69, wherein said hydrogel comprises a hydrogel formed from one or more materials selected from the group consisting of poly(N-isopropylacrylamide) (PNIPA), poly(N-isopropylacrylamide-co-1-vinylimidazole) (PNIPA-VI), poly(acrylamide) (PAAm), poly(acrylamide), poly(N,N-dimethylacrylamide), poly(N,N-diethylacrylamide), poly(1-vinylimidazole), poly(sodium acrylate), poly(sodium methacrylate), poly(2-hydroxyethylmethacrylate) (HEMA), poly(N,N-dimethylaminoethyl methacrylate) (DMAEMA), poly(N-[tris(hydroxymethyl)methyl]acrylamide), poly(1-(3-methacryloxy)propylsulfonic acid) (sodium salt), poly(allylamine), poly(N-acryloxysuccinimide), poly(N-vinylcaprolactam), poly(1-vinyl-2-pyrrolidone), poly(2-acrylamido-2-methyl-1-propanesulfonic acid) (sodium salt), poly((3-acrylamidopropyl) trimethylammonium chloride), and poly(diallyldimethylammonium chloride).

Embodiment 71: The method of embodiment 53, wherein said nanoparticle comprises a metal organic framework (MOF).

Embodiment 72: The method of embodiment 71, wherein said nanoparticle comprises a metal organic framework selected from the group consisting of zeolitic imidazolate frameworks (ZIFs), Universitetet i Oslo (University of Oslo) frameworks (UiOs), and (Materials of Institut Lavoisier frameworks (MILs).

Embodiment 73: The method of embodiment 72, wherein said nanoparticle comprises a metal organic framework selected from the group consisting of ZIF-8, ZIF-67, ZIF-90, Fe-BTC, HKUST-1, and MIL-53, MIL-89, MIL-88A, MIL-100, UiO-66, UiO-66-NH₂, MOF-801, MOF-804, Fe-NDC-M, MOF-1201, MOF-1203, and Fe-NDC-O MOFs.

Embodiment 74: The method of embodiment 73, wherein said nanoparticle comprises a MIL-88A MOF.

Embodiment 75: The method of embodiment 73, wherein said nanoparticle comprises a ZIF-8 MOF.

Embodiment 76: The method of embodiment 73, wherein said nanoparticle comprises a UiO-66 MOF, or a UiO-66-NH₂ MOF.

Embodiment 77: The method according to any one of embodiments 1-76, wherein said drug delivery vehicles have an average hydrodynamic diameter ranging from about 30 nm or about 50 nm up to about 300 nm, or from about 30 nm or about 50 nm up to about 200 nm, or from about 30 nm or about 50 nm up to about 170 nm, or from about 30 nm or about 50 nm up to about 150 nm, or from about 30 nm or about 50 nm up to about 100 nm, or from about 30 nm or about 50 nm up to about 80 nm, or from about 30 nm or about 50 nm up to about 70 nm, or from about 60 nm up to about 70 nm by DLS.

Embodiment 78: The method of embodiment 77, wherein said drug delivery vehicles have an average hydrodynamic diameter ranging from about 145 nm up to about 165 nm by DLS or from about 150 nm up to about 161 nm by DLS.

Embodiment 79: The method according to any one of embodiments 1-78, wherein said nanoparticle has an average pore size that ranges from about 1 to about 20 nm, or from about 1 to about 10 nm, or from about 1 to about 5 nm, or from about 1 to about 4 nm, or from about 1 to about 3 nm, or from about 2 to about 3 nm.

Embodiment 80: The method according to any one of embodiments 1-79, wherein said lipid bilayer comprises a phospholipid, and cholesterol (CHOL) and/or a cholesterol derivative.

Embodiment 81: The method of embodiment 80, wherein said lipid bilayer comprises a phospholipid and cholesterol (CHOL).

Embodiment 82: The method according to any one of embodiments 80-81, wherein said phospholipid comprises a saturated fatty acid with a C14-C20 carbon chain, and/or an unsaturated fatty acid with a C14-C20 carbon chain, and/or a natural lipid comprising a mixture of fatty acids with C12-C20 carbon chains.

Embodiment 83: The method of embodiment 82, wherein said phospholipid comprises one or more phospholipids selected from the group consisting of distearoylphosphatidylcholine (DSPC), phosphatidylcholine (DPPC), 1,2-dimyristoleoyl-sn-glycero-3-phosphocholine (DMPC), 1,2-dioleoyl-sn-glycero-3-phosphocholine (DOPC), 1,2-distearoyl-sn-glycero-3-phospho-rac-glycerol (DSPG), 1,2-dipalmitoyl-sn-glycero-3-phosphoglycerol (DPPG), 1,2-dieicosenoyl-sn-glycero-3-phosphocholine, and diactylphosphatidylcholine (DAPC), 1, 2-Distearoyl-sn-glycero-3-phosphoethanolamine (DSPE), and dipalmitoyl phosphatidylethanolamine.

Embodiment 84: The method of embodiment 82, wherein said phospholipid comprises a natural lipid selected from the group consisting of egg phosphatidylcholine (egg PC), and soy phosphatidylcholine (soy PC).

Embodiment 85: The method of embodiment 82, wherein said phospholipid comprises distearoylphosphatidylcholine (DSPC).

Embodiment 86: The method according to any one of embodiments 80-85, wherein said lipid bilayer comprises an mPEG phospholipid with a phospholipid C14-C18 carbon chain, and a PEG molecular weight ranging from about 350 Da to 5000 Da.

Embodiment 87: The method of embodiment 86, wherein said lipid bilayer comprises 1, 2-Distearoyl-sn-glycero-3-phosphoethanolamine-Poly(ethylene glycol) (DSP-PEG), or dipalmitoyl phosphatidylethanolamine grafted poly(ethylene glycol) (PE-PEG).

Embodiment 88: The method of embodiment 87, wherein said PE-PEG comprises DS PE-PEG_{2K}.

Embodiment 89: The method of embodiment 87, wherein said PE-PEG comprises DSPE-PEG_{5K}.

Embodiment 90: The method according to any one of embodiments 85-89, wherein said lipid bilayer comprises DPSC, cholesterol, and DSPE-PEG.

Embodiment 91: The method of embodiment 90, wherein the molar ratio of DPSC : cholesterol : PE-PEG ranges from 20-90% DSPC : 10%-50% Chol : 1%-10% DS PE-PEG.

Embodiment 92: The method of embodiment 91, wherein the molar ratio of DSPC : Chol :DS PE-PEG is about 3 : 2 : 0.15.

Embodiment 93: The method according to any one of embodiments 80-92, wherein said nanoparticles have a particle (e.g., MSNP):lipid ratio of 1:1.25 (w/w) or greater.

Embodiment 94: The method according to any one of embodiments 80-92, wherein said lipid bilayer comprises a cholesterol derivative selected from the group consisting of cholesterol hemisuccinate (CHEMS), lysine-based cholesterol (CHLYS), and PEGylated cholesterol (Chol-PEG).

Embodiment 95: The method of embodiment 94, wherein said cholesterol derivative is in place of said cholesterol.

Embodiment 96: The method according to any one of embodiments 94-95, wherein said lipid bilayer comprises CHEMS.

Embodiment 97: The method of embodiment 96, wherein said lipid bilayer comprises CHEMS ranging from about 5% (mol percent) up to about 30% total lipid.

Embodiment 98: The method of embodiment 97, wherein said lipid bilayer comprises about 10% or about 20% CHEMS or about 30% CHEMS or about 40% CHEMS.

Embodiment 99: The method according to any one of embodiments 1-98, wherein said one or more camptothecin analogs, and/or one or more autophagy inhibitors are loaded into said nanoparticle with a cargo trapping agent (e.g., protonating agent).

Embodiment 100: The method of embodiment 99, wherein said cargo trapping agent before reaction with the one or more camptothecin analogs, and/or one or more autophagy inhibitors is selected from the group consisting of triethylammonium sucrose octasulfate (TEA₈SOS), citric acid, (NH₄)₂SO₄, an ammonium salt, a trimethylammonium salt, and a triethylammonium salt.

Embodiment 101: The method according to any one of embodiments 1-100, wherein said drug delivery vehicle is conjugated to a moiety selected from the group consisting of a targeting moiety, a fusogenic peptide, and a transport peptide.

Embodiment 102: The method of embodiment 101, wherein said drug delivery vehicle is conjugated to a peptide that binds a receptor on a cancer cell or tumor blood vessel.

Embodiment 103: The method of embodiment 102, wherein said drug delivery vehicle is conjugated to an iRGD peptide.

Embodiment 104: The method of embodiment 102, wherein said drug delivery vehicle is conjugated to a targeting ligand shown in Table 4.

Embodiment 105: The method according to any one of embodiments 101-104, wherein said drug delivery vehicle is conjugated to transferrin, and/or ApoE, and/or folate.

Embodiment 106: The method according to any one of embodiments 101-105, wherein said drug delivery vehicle is conjugated to a targeting moiety that comprises an antibody that binds to a cancer marker.

Embodiment 107: The method of embodiment 106, wherein said drug delivery vehicle is conjugated to a targeting moiety that comprises an antibody that binds a cancer marker shown in Table 3.

Embodiment 108: The method according to any one of embodiments 106-107, wherein said antibody is selected from the group consisting of an intact immunoglobulin, an F(ab)′₂, a Fab, a single chain antibody, a diabody, an affibody, a unibody, and a nanobody.

Embodiment 109: The method according to any one of embodiments 1-108, wherein said drug delivery vehicles in suspension are stable for at least 1 month, or at least 2 months, or at least 3 months, or at least 4 months, or at least 5 months, or at least 6 months when stored at 4° C.

Embodiment 110: The method according to any one of embodiments 1-109, wherein said drug delivery vehicles form a stable suspension on rehydration after lyophilization.

Embodiment 111: The method according to any one of embodiments 1-110, wherein said methods, show reduced drug toxicity as compared to free one or more camptothecin analogs alone or in combination with said one or more autophagy inhibitors.

Embodiment 112: The method according to any one of embodiments 1-111, wherein said drug delivery vehicles have colloidal stability in physiological fluids with pH 7.4 and remains monodisperse to allow systemic biodistribution and are capable of entering a disease site by vascular leakage (EPR effect) or transcytosis.

Embodiment 113: The method drug carrier according to any one of embodiments 1-112, wherein said drug delivery vehicles are colloidally stable.

Embodiment 114: The method according to any one of embodiments 1-113, wherein said method comprises a component of a primary therapy in a chemotherapeutic regimen.

Embodiment 115: The method according to any one of embodiments 1-113, wherein said method comprises an adjunct therapy in a treatment regime that additionally comprises chemotherapy using another chemotherapeutic agent, and/or surgical resection of a tumor mass, and/or radiotherapy.

Embodiment 116: The method according to any one of embodiments 1-115, wherein said cancer comprises a solid tumor.

Embodiment 117: The method of embodiment 116, wherein said cancer comprises a cancer selected from the group consisting of pancreatic cancer, gastric cancer, hepatocellular carcinoma, head and neck squamous cell carcinoma, urothelial carcinoma, cervical cancer, non-small cell lung cancer, and broadly for non-respectable solid tumors with high microsatellite instability (MSI-H) or DNA mismatch repair deficiency.

Embodiment 118: The method according to any one of embodiments 114-117, wherein said cancer comprises pancreatic cancer.

Embodiment 119: The method according to any one of embodiments 114-117, wherein said cancer comprises colorectal cancer.

Embodiment 120: The method according to any one of embodiments 114-117, wherein said cancer comprises lung cancer.

Embodiment 121: The method according to any one of embodiments 114-117, wherein said cancer is a cancer selected from the group consisting of breast cancer, lung cancer, melanoma, pancreas cancer, liver cancer, acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm’s tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm’s tumor.

Embodiment 122: The method according to any one of embodiments 1-121, wherein administration of said one or more checkpoint inhibitor(s), and/or said one or more camptothecin analogs, and/or said one or more autophagy inhibitors is via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

Embodiment 123: The method according to any one of embodiments 1-121, wherein administration of said one or more checkpoint inhibitor(s), and/or said one or more camptothecin analogs, and/or said one or more autophagy inhibitors comprises systemic administration via injection or cannula.

Embodiment 124: The method according to any one of embodiments 1-121, wherein administration of said one or more checkpoint inhibitor(s), and/or said one or more camptothecin analogs, and/or said one or more autophagy inhibitors comprises administration to an intra-tumoral or peri-tumoral site.

Embodiment 125: The method according to any one of embodiments 1-124, wherein said mammal is a human.

Embodiment 126: The method according to any one of embodiments 1-124, wherein said mammal is a non-human mammal.

Embodiment 127: A pharmaceutical formulation comprising:
nanoparticle drug carrier according to any one of embodiments 1-113;
a checkpoint inhibitor; and
a pharmaceutically acceptable carrier.

Embodiment 128: The pharmaceutical formulation of embodiment 127, wherein said formulation is an emulsion, dispersion, or suspension.

Embodiment 129: The pharmaceutical formulation of embodiment 128, wherein said suspension, emulsion, or dispersion is stable for at least 1 month, or at least 2 months, or at least 3 months, or at least 4 months, or at least 5 months, or at least 6 months when stored at 4° C.

Embodiment 130: The pharmaceutical formulation according to any one of embodiments 127-129, wherein the nanovesicle drug carriers, and/or the a nanoparticle drug carriers, and/or the a nanomaterial carriers in said formulation show a substantially unimodal size distribution; and/or show a PDI less than about 0.2, or less than about 0.1.

Embodiment 131: The pharmaceutical formulation according to any one of embodiments 127-130, wherein said formulation is formulated for administration via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

Embodiment 132: The pharmaceutical formulation according to any one of embodiments 127-130, wherein said formulation is a sterile injectable.

Embodiment 133: The pharmaceutical formulation according to any one of embodiments 127-132, wherein said formulation is a unit dosage formulation.

### DEFINITIONS

The terms “subject,” “individual,” and “patient” may be used interchangeably and refer to humans, as well as non-human mammals (e.g., non-human primates, rodentia, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for a cancer as described herein. Thus, for example, in certain embodiments the subject is a subject with a cancer (e.g., pancreatic ductal adenocarcinoma (PDAC), breast cancer (e.g., drug-resistant breast cancer), colon cancer, brain cancer, and the like). In certain embodiments the methods described herein are prophylactic and the subject is one in whom a cancer is to be inhibited or prevented. In certain embodiments the subject for prophylaxis is one with a family history of cancer and/or a risk factor for a cancer (e.g., a genetic risk factor, an environmental exposure, and the like).

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term “treat” can refer to prophylactic treatment which includes a delay in the onset or the prevention of the onset of a pathology or disease.

The terms “coadministration” or “administration in conjunction with” or “cotreatment” when used in reference to the coadministration of a first compound (or component) (e.g., a camptothecin analog) and a second compound (or component) (e.g., a checkpoint inhibitor) indicates that the first compound (or component) and the second compound (or component) are administered so that there is at least some chronological overlap in the biological activity of first compound and the second compound in the organism to which they are administered. Coadministration can include simultaneous administration or sequential administration. In sequential administration there may even be some substantial delay (e.g., minutes or even hours) between administration of the first compound and the second compound as long as their biological activities overlap. In certain embodiments, the coadminstration is over a time frame that permits the first compound and second compound to produce an enhanced therapeutic or prophylactic effect on the organism. In certain embodiments the enhanced effect is a synergistic effect. Where reference is made to administering a first drug and a second drug it will be understood that the first drug and second drug are administered “in conjunction” with each other as defined above.

The terms “nanocarrier” and “nanoparticle drug carrier” are used interchangeably and refer to a nanostructure having a one or a plurality of cavities, e.g., a porous interior. In various embodiments, the cavities contain a cargo that is to be delivered, e.g., to a target cell. In certain embodiments the nanoparticle is a porous silica nanoparticle (e.g., mesoporous silica nanoparticle or “MSNP”), or other porous particle. In certain other embodiments, the nanoparticle is a solid nanoparticle and the cargo can be disposed within (e.g., intermixed with) the material forming the nanoparticle or adsorbed to, or covalently or ionically bound to, the nanoparticle surface). In certain embodiments the nanocarrier comprises a lipid bilayer encasing (or surrounding or enveloping) the particle core. In certain embodiments the nanocarrier is a liposome and the cargo can be disposed within the liposome.

As used herein, the term “lipid” refers to conventional lipids, phospholipids, cholesterol, chemically functionalized lipids for attachment of PEG, pharmaceutically active ingredients, ligands, etc.

As used herein, the terms “lipid bilayer” or “LB” refers to any double layer of oriented amphipathic lipid molecules in which the hydrocarbon tails face inward to form a continuous non-polar phase.

As used herein, the term “selective targeting” or “specific binding” refers to use of targeting ligands on the surface of a drug delivery nanocarrier (e.g., a LB-coated nanoparticle or a liposome). In certain embodiments the targeting ligand(s) are on the surface of a lipid bilayer or LB-coated nanoparticle. Typically, the ligands interact specifically/selectively with receptors or other biomolecular components expressed on the target, e.g., a cell surface of interest. The targeting ligands can include such molecules and/or materials as peptides, antibodies, aptamers, targeting peptides, polysaccharides, and the like.

A coated mesoporous silica nanoparticle, having targeting ligands can be referred to as a targeted nanoparticle or a targeted drug delivery nanocarrier (e.g., LB-coated nanoparticle or a liposome).

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. For example, “about” can mean within 1 or more than 1 standard deviation, per the practice in the art. Alternatively, “about” can mean a range of up to 20%, preferably up to 10%, more preferably up to 5% and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

A “pharmaceutically acceptable carrier” as used herein is defined as any of the standard pharmaceutically acceptable carriers. The pharmaceutical compositions of the subject invention can be formulated according to known methods for preparing pharmaceutically useful compositions. The pharmaceutically acceptable carrier can include diluents, adjuvants, and vehicles, as well as carriers, and inert, non-toxic solid or liquid fillers, diluents, or encapsulating material that does not react with the active ingredients of the invention. Examples include, but are not limited to: phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington’s Pharmaceutical Sciences (Martin E W (1995) Easton Pa., Mack Publishing Company, 19th ed.) describes formulations that can be used in connection with the drug delivery nanocarrier(s) (e.g., liposomes or nanoparticles encapsulated with a lipid bilayer) described herein.

As used herein, an “antibody” refers to a protein consisting of one or more polypeptides substantially encoded by immunoglobulin genes or fragments of immunoglobulin genes or derived therefrom that is capable of binding (e.g., specifically binding) to a target (e.g., to a target polypeptide). The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W.E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Certain preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H-}V_{L} heterodimer which may be expressed from a nucleic acid including V_{H}- and V_{L}- encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv’s (scFv), however, alternative expression strategies have also been successful. For example Fab^{I} molecules can be displayed on a phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No: 5733743). The scFv antibodies and a number of other structures converting the naturally aggregated, but chemically separated light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778). In certain embodiments antibodies should include all that have been displayed on phage (e.g., scFv, Fv, Fab and disulfide linked Fv (see, e.g., Reiter et al. (1995) Protein Eng. 8: 1323-1331) as well as affibodies, unibodies, and the like.

The term “specifically binds”, as used herein, when referring to a biomolecule (e.g., protein, nucleic acid, antibody, etc.), refers to a binding reaction that is determinative of the presence of a biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The phrase “cause to be administered” refers to the actions taken by a medical professional (e.g., a physician), or a person prescribing and/or controlling medical care of a subject, that control and/or determine, and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

### BRIEF DESCRIPTION OF THE DRAWINGS

FIG. 1, panels A-F, shows that the alkalizing effect of free IRIN leads to autophagy inhibition and upregulation of PD-L1 expression in KPC cells. Panel A) IRIN, a major PDAC cancer drug, is a weak base that can be protonated in an acidic environment. Panel (B) Confocal microscopy to demonstrate the localization of the amphiphilic drug, in organelles close to the surface membrane of KPC cells, exposed to 300 µM IRIN for 24 h. The drug exhibits blue fluorescence at an excitation wavelength of 405 nm. The cell membrane was stained by ALEXA FLUOR® 594 conjugated WGA (red). Bar:10 µm. Panel (C) Representative confocal microscopy to demonstrate that IRIN (300 µM, 24 h) could neutralize the acidic pH of lysosomes that were stained by the red fluorescent acidotropic dye, DND 99 Lysotracker. Alkalization of these organelles by IRIN resulted in a sharp reduction of DND 99 fluorescence, which is overtaken by the blue fluorescence of the drug in the same compartment. Co-staining with Hoechst 33342 showed the presence of nuclear condensation in IRIN-treated cells. Bar: 10 µm. Panel D) Dose- and time-dependent study of the lysosomal alkalization effect of free IRIN at the indicated concentrations (left) and incubation time periods (right). Image J software analysis was used to quantify the change in DND 99 fluorescence intensity. Data represents mean ± SD, n=3. *, p<0.05; ***, p<0.001 (1-way ANOVA followed by a Tukey’s test). The corresponding confocal images appear in FIG. 9, panel E) IF staining of LC3B, p62 and PD-L1 in KPC cells exposed to IRIN (300 µM), CQ (32 µM), RAP (100 nM) or IFN-γ (10 ng/mL) for 24 h. Bar is 10 µm. Panel F) Immunoblotting of LC3 and PD-L1 in KPC lysates following cellular treatment with IRIN at the indicated concentrations for 24 h. Densitometric analysis was performed by ImageJ software and the fold of intensity was normalized to vinculin.

FIG. 2, panels A-D, shows an assessment of ER stress responses induced by free IRIN in KPC cells. Panel A) Left: Simplified schematic to show the unfolded protein stress response in the ER. Right: immunoblotting to show the expression of the ER stress response marker CHOP and cleaved caspase 3 (CC-3) in KPC cells treated with IRIN for 24 h. Panel B) CRT expression was assessed by flow cytometer (left panel), while HMGB1 release was determined by ELSLA (right panel) in KPC cells exposed to OX (500 µM), IRIN (300 µM), DOX (20 µM) and PTX (12 µM) for 24 h. Data are expressed as mean ± SD. n =3. *, p<0.05; **, p<0.01; ***, p<0.001 compared to PBS control (Student’s t-test). Panel C) Vaccination experiment in a PDAC mouse model. Left: The schematic shows execution of the vaccination study through subcutaneous injection of dying KPC cells treated with IRIN or OX, followed by re-challenge with untreated KPC cells. As a negative control in the vaccination experiment, mice were treated with PBS only, without cellular debris. Right: Tumor growth curves of normal KPC cells, injected in the opposite flank of the vaccinated animals. Data are expressed as mean ± SEM. n=6. Insert: Photography of the harvested tumors collected for each group. Note that there was one tumor free animal in the IRIN group (labeled as “Ⓧ”). Bar: 2 cm. Panel D) Quantitative assessment of CD8⁺/FoxP3⁺ cell ratios by IHC analysis. *, p<0.05; **, p<0.01 (1-way ANOVA followed by a Tukey’s test).

FIG. 3, panels A-C, illustrates silicasome synthesis and assessment of lysosomal pH, ER stress and autophagy in KPC cells, using encapsulated IRIN. Panel A) Schematic to explain large batch synthesis and characterization of the silicasome nanocarrier in this study. The carrier is comprised of a MSNP core, which contains a large packaging space for IRIN loading, and encapsulation by a lipid bilayer (comprised of DSPC: Cholesterol: PE-PEG_{2K} at 3:2:0.15 molar ratio). The fully synthesized carrier was dispensed into vials containing 50 mg IRIN/container. CryoEM was undertaken to show particle morphology, in addition to characterization of size, charge and IRIN loading capacity, as shown. Panel B) KPC cells were treated with IRIN silicasome at indicated concentrations for 24 hrs. Empty silicasomes (equivalent to 300 µM IRIN) was included as control. The lysosome alkalizing effect was studied with the DND99 dye, similar to FIG. 1, panel C. Panel C) KPC cells were treated by free IRIN and IRIN silicasome at drug concentration of 300 µM for 24 hrs. Empty. Silicasomes were used as control. The treated cells were used for further analysis by LC3-II/I, p62 and PD-L1 immunoblotting, as described in FIG. 1, panel F. Image J software was used to quantify the fluorescent intensity and band density. Data represent mean ± SD, n=3. ***, p<0.001 (1-way ANOVA followed by a Tukey’s test).

FIG. 4, panels A-B, shows results of an animal survival study in an orthotopic KPC model, treated with an IRIN silicasome plus anti-PD-1 antibody. Panel A) Explanation of the KPC model, including orthotopic implant in the pancreas and technical development of the primary tumor and metastases that can be followed by IVIS imaging. Animals were sacrificed according to the moribund criteria shown. Panel B). Details of the survival experiment in tumor-bearing mice (n = 5-7), which were treated with free IRIN or the silicasome at an IRIN dose equivalent of 40 mg/kg IV every 3- or 4-days, with or without IP administration of 100 µg anti-PD-1 antibody, for a total of six administrations. Please notice that the antibody was administered two days after IRIN injection. Saline and anti-PD-1 antibody alone were used as controls. Kaplan-Meier plots were used to display the survival rate of the different animal groups (*p<0.05, Log Rank test).

FIG. 5, panels A-D, shows the results of an efficacy study in the KPC model to demonstrate the generation of ICD markers by the IRIN silicasome. Panel A) This experiment was undertaken to demonstrate the immunogenic effect of IRIN in orthotopic tumor-bearing mice received 3 IV injections of either free IRIN or the silicasome at a drug dose of 40 mg/kg, followed by animal sacrifice 72 h after the last treatment. IVIS imaging was performed on explanted organs to obtain the bioluminescence intensity in the region of the primary tumor as well as the metastases, which were quantitatively displayed by IVIS software in the left panel as normalized value. Panels B and C) IHC analysis to determine CRT (panel B) and LC3B (panel C) expression at the orthotopic tumor site. Imaging intensity was quantitatively expressed as fold increase that was normalized to the saline group. Representative IHC images are shown on the right. Bar is 50 µm. Panel D) Quantitative assessment of HMGB 1 release. The IHC images were analyzed by Aperio ImageScope software to determine protein released from the damaged nuclei, as described in FIG. 22. Data are expressed as mean ± SEM, n=3. *, p<0.05; **, p<0.01; ***, p<0.001 (1-way ANOVA followed by a Tukey’s test).

FIG. 6, panels A-E, shows an analysis of cognate immunity in the efficacy experiment in FIG. 5. Additional IHC analysis was undertaken using the tumor samples collected in FIG. 5, panel A. Quantitative assessment of: Panel A) the CD8⁺/ FoxP3⁺ cell ratio; Panel B), perforin; Panel C) granzyme B; Panel D) IFN-γ production; and Panel E) PD-L1 expression. Representative IHC images appear in FIG. 18, panels A-C. Data are expressed as mean ± SEM, n=3. *, p<0.05; **, p<0.01; ***, p<0.001 (1-way ANOVA followed by a Tukey’s test).

FIG. 7 shows the results of a comparative anti-tumor immune response for IRIN delivery by the silicasome vs ONIVYDE®, w/wo anti-PD-1. The treatment schedule is outlined in the upper panel. Orthotopic KPC tumor-bearing mice (n = 6) were IV injected with ONIVYDE® or the silicasome at an IRIN dose of 40 mg/kg every 3~4 days, w/wo anti-PD-1 (100 µg/mouse) IP, two days later. The controls included saline and anti-PD-1 antibody alone. Kaplan-Meier plots were used to display the differential survival rate of the different treatment groups (*p<0.05, Log Rank test).

FIG. 8 provides a schematic to show the synthesis steps towards the production of a large batch of IRIN-loaded silicasome nanoparticles. These steps include: (1) synthesis of bare mesoporous silica nanoparticles (MSNP) synthesis at 20 L scale, using a sol-gel reaction as previously described¹; (2) lipid coating after soaking in the trapping agent triethylammonium sucrose octasulfate (TEA₈SOS), through the use of continuous flow cell sonication procedure; and (3) remote loading of irinotecan, followed by column purification and sterilization. Box 1: An in-house equipment setup of the flow cell sonication system. Box 2: Schematic to show the mechanism of irinotecan remote loading. Silicasome particles containing the trapping agent were incubated in an IRIN solution, allowing the amphipathic drug to diffuse across the lipid bilayer. Proton release from the trapping agent converted the encapsulated IRIN to a hydrophilic derivative that cannot back-diffuse across the LB. The protonated drug interacts with negatively charged SOS⁸⁻ to form a drug precipitate (Liu et al. (2019) ACS Nano, 13: 38-53; Liu et al. (2016) ACS Nano, 10: 2702-2715).

FIG. 9, panels A-D, illustrates a dose- and time-dependent study on IRIN-induced lysosomal alkalization effect in KPC cells. Panel A) Dose-dependence. KPC cells were treated with indicated free IRIN dose for 24 hours, followed by DND 99 and Hoechst dye co-staining, similar to FIG. 1, panel C. We were able to discern a major signal drop at 20 µM. IRIN accumulation in the KPC cells is indicated by white arrows. Panel B) Time-dependence. KPC cells were treated using 300 µM free IRIN for indicated time period, followed by DND 99/Hoechst co-staining, similar to panel A. We were able to observe a major DND 99 signal drop as early as 1 h post incubation. Quantification of DND 99 fluorescence intensity in panels A and B higher presented in FIG. 1, panel D. Panel C) Similar to the experiment described in FIG. 1, panel C, KPC cells were exposed to IRIN (300 µM) or chloroquine (CQ, 32 µM) for 24 h. The disappearance of the red fluorescence staining in the acidifying organellar environment in IRIN- and CQ-treated cells is as a result of the alkalization effect of these weak-base molecules. Bars are 10 µm. Panel D) The Henderson-Hasselbalch (H—H) equation allows calculation of % protonated drug at different pH values. Although 83.3% of IRIN molecules are protonated at pH 7.4, the equilibrium shifts to ~99% of the molecules at a lysosomal relevant pH ~4.5-5.5. This equilibrium shift is still sufficient for depleting acidic the available protons in the lysosome, allowing alkalization to occur. It is possible to achieve this effect with an IRIN dose as low as ~20 µM (A).

FIG. 10, panels A-B, shows a dose- and time-dependent study on IRIN induced changes on LC3B, p62 and PD-L1 in KPC cells. Panel A) KPC cells were treated with indicated free IRIN doses for 24 hours, followed by immunofluorescence (IF) staining, similar to FIG. 1, panel E. Panel B) KPC cells were treated using 300 µM free IRIN for indicated time, followed by IF staining of LC3B, p62 and PD-L1. Bars represent 10 µm. Signal intensity was quantified by Image J software. At least three representative images were analyzed for each treatment. Data represents mean ± SD, n = 3. *p<0.05, **p<0.01, ***p<0.001 (1-way ANOVA followed by a Tukey’s test).

FIG. 11 shows that IRIN treatment leads to the phosphorylation of the p65 NF-κB subunit in KPC cells. KPC cells were treated by free IRIN (300 µM) for 24 hrs, followed by immunoblotting assay for total and phosphorylated p65 NF-κB subunit. Vinculin was included to normalize the intensity of p-p65 abundance.

FIG. 12 shows that irinotecan, but not oxaliplatin, inhibited autophagy flux in KPC cells. KPC cells were treated with IRIN (300 µM) or oxaliplatin (OX, 500 µM) for 24 h. This was followed by a Western blotting analysis for expression of the autophagy markers, LC3 and p62. Densitometric analysis was performed by Image J software and the fold-intensity was normalized to that of housekeeping protein, β-Actin. Data represents mean ± SD, n=3. *p<0.05 compared to PBS group, ^{#}p<0.05 compared to OX group (1-way ANOVA followed by a Tukey’s test).

FIG. 13 shows that irinotecan, but not oxaliplatin, induced PD-L1 upregulation in KPC cells. KPC cells were treated with IRIN (300 µM) or OX (500 µM) for 24 h. PBS treatment was used as a negative control. PD-L1 expression was assayed using immunoblotting. Densitometric analysis was performed and normalized according to the expression levels of the housekeeping protein, vinculin. Data represents mean ± SD, n=3. *p<0.05 compared to PBS group, ^{#}p<0.05 compared to OX group (1-way ANOVA followed by a Tukey’s test).

FIG. 14 shows the results of fluorescence microscopy to demonstrate the generation of reactive oxygen species (ROS) in KPC cells. Intracellular ROS production was determined by a fluorescence-based ROS Kit (Abcam, ab186027) in KPC cells. This assay measures total ROS species according to vendor’s instructions. This assay is frequently used in cell biology to show the contribution of ROS in the context of ER stress and autophagy³. Briefly, KPC cells were treated with IRIN (300 µM), OX (500 µM) or TUN (10 µM) for 24 h. Images were captured using a fluorescence microscope. Signal intensity was quantified by Image J software. At least three representative images were analyzed for each treatment. Data represents mean ± SD, n=3. *p<0.05, ***p<0.001, compared to PBS group (1-way ANOVA followed by a Tukey’s test). Bar is 50 µm.

FIG. 15 shows the results of fluorescence microscopy to demonstrate intracellular Ca²⁺ release in KPC cells. Intracellular Ca²⁺ flux was assessed by a Fluo 4 AM assay in KPC cells. The cells were treated with IRIN (300 µM), OX (500 µM) or TUN (10 µM) for 24 h. Images were captured using a fluorescence microscope, followed by data analysis using the Image J software. At least three representative images were analyzed for each treatment. Data represents mean ± SD, n=3. **p<0.01, ***p<0.001, compared to PBS group (1-way ANOVA followed by a Tukey’s test). Bar is 50 µm.

FIG. 16, panels A-B, illustrates an assessment of ecto-CRT expression in KPC cells. Panel A) Dose-dependent CRT expression in KPC cells treated by IRIN at different concentrations for 24 h. CRT expression was determined by flow cytometry as we described in the method section. Data represents mean ± SD (n = 3). *p<0.05, ** p<0.001 compared to PBS group (1-way ANOVA followed by a Tukey’s test). Panel B) Confocal microscopy showing the appearance of CRT on the KPC cell surface of the cells treated with IRIN (300 µM) for 24 h. Bar is 20 µm. Green: CRT; Blue: Nuclear.

FIG. 17 shows the results of IHC staining of CD8⁺ and FoxP3⁺ T cells in the vaccination experiment, as we described in the FIG. 2, panel D. Left panel: Quantification of CD8⁺ cytotoxic T cells and FoxP3⁺ regulatory T cells (Treg) from IHC staining. Data represents mean ± SEM (n=6), *p<0.05, **p<0.01, ***p<0.001 (1-way ANOVA followed by a Tukey’s test). Right panel: Representative IHC images were shown. Bars are 50 µm. While there was no significant increase in CD8⁺ staining number, IRIN significantly (p<0.01) decreased FoxP3⁺ expression, which led to a significant increase of CD8⁺/ FoxP3⁺ ratio as reflected in FIG. 2, panel D.

FIG. 18 shows the results of IF staining of LC3B, p62 and PD-L1 in KPC cells treated with the silicasome w/wo IRIN. KPC cells were treated with IRIN silicasome at drug concentration of 300 µM for 24 hours. Empty silicasome (500 µg/mL, equivalent to drug dose of 300 µM) and PBS were used as control. IF staining of LC3B, p62 and PD-L1 was performed similar to FIG. 10. Bar is 10 µm.

FIG. 19, panels A-C shows that IRIN silicasome induced ER stress in KPC cells. KPC cells were treated with IRIN silicasome at drug dose of 75 µM and 300 µM for 24 hours. PBS and empty silicasome (that is equivalent to drug dose of 300 µM) were included as control. Ca²⁺ efflux (panel A) and total ROS (panel B) measurements were performed similar to FIGS. 14 and 15. Data represents mean ± SD, n=3. *p<0.05, **p<0.001, n.s., not significant (1-way ANOVA followed by a Tukey’s test). Panel C) In a separate experiment, KPC cells were seeded in to a 6-well plate, followed by the treatment using silicasome w/wo IRIN (300 µM) for 48 hours. ER marker, CHOP protein, was assayed by western blotting similar to FIG. 2, panel A

FIG. 20, panels A-C, shows the results of confirmative cellular studies in PANC-1 cells. To confirm the effect of IRIN, another PDAC cell line, i.e. PANC-1 cells, were used to study IRIN induced alkalization effect (panel A), autophagy inhibition (panel B) and PD-L1 induction (panel C). The treatments included free IRIN, IRIN silicasome and empty silicasome at equivalent drug concentration of 300 µM for 24 hrs. PBS served as a negative control. Bars: 10 µm. Signal intensity was quantified by Image J. At least three representative images were analyzed for each treatment. Data represents mean ± SD, n = 3. *p<0.05, **p<0.01, ***p<0.001 (1-way ANOVA followed by a Tukey’s test). Consistent to the KPC data, both free and encapsulated IRIN were capable of neutralizing lysosomal pH, triggered LC-3B generation and increase PD-L1 expression in PANC-1 cells. No significant effect was observed when empty silicasome was tested.

FIG. 21 illustrates tumor weight measurement for the efficacy study shown in FIG. 5, panel A. The animal treatment is described. In addition to the IVIS imaging and ROI analysis that were described, the excised tumor tissues were weighed at the time of sacrifice. Data represents mean ± SEM (n=3), *p<0.05.

FIG. 22, panels A-B, shows HMGB1 staining and software assisted quantification of the amount of HMGB 1 released from the nucleus. Panel A) Use of Aperio ImageScope software to quantify HMGB1 release in tumor tissues receiving different treatments. High resolution HMGB1 IHC pictures were scanned, followed by a software mediated imaging analysis process, which can discern “pixel density” in the picture. While the strong positive pixel density comes from the nuclear region (non-released HMGB1), the weak- or mid-positive regions come from the released HMGB1. The % of HMGB1 release was calculated by [(weak-positive + mid-positive pixel counts) / (total positive pixel counts)] x 100%. Panel B) Representative IHC images of HMGB1 staining in each treatment groups (FIG. 5, panel D). Bar is 50 µm.

FIG. 23, shows the results of IHC staining of CD8⁺ and FoxP3⁺ T cells in the efficacy experiment presented in the FIG. 5. Left panel: Quantification of CD8⁺ cytotoxic T cells and FoxP3⁺ Treg cells from the IHC staining. Data represents mean ± SEM (n=3), **p<0.01 (1-way ANOVA followed by a Tukey’s test). Right panel: Representative IHC images were shown. Bars are 50 µm. While there was marginal effect of increasing CD8⁺ number in response to IRIN silicasome, this treatment significantly decreased FoxP3⁺ expression (p <0.01), which led to significant increase of CD8⁺/ FoxP3⁺ ratio as reflected in FIG. 6. panel A.

FIG. 24, panels A-C, shows representative images of IHC staining of panel A) perforin and granzyme, panel B) IFN-γ and panel C) PD-L1 in orthotopic KPC tumors in the efficacy experiment (FIGS. 5 and 6). Bars are 50 µm.

FIG. 25 shows drug content at the orthotopic KPC tumor site after 24 h in animals receiving an IV injection of 40 mg/kg free irinotecan or IRIN silicasome. The irinotecan concentration was measured by LC-MS (Waters LCT Premier ESI). Data represent mean ± SD, N=3. ***p<0.001 by Student’s t-test.

FIG. 26 shows IRIN release from silicasome at different pHs under abiotic conditions. IRIN laden silicasomes (100 µg/mL IRIN) were suspended in a 10 mM phosphonate buffer at pH 7.4 or pH 4.5, respectively. The suspension was incubated at 37° C. with shaking. At the indicated time period, 400 µL the particles suspension was centrifugated at 15 K rpm for 10 min, following which the released drug in the supernatant was analyzed through UV absorption at 360 nm. Data represent mean ± SD, n=3.

### DETAILED DESCRIPTION

As described in Example 1, the use of lipid bilayer coated nanoparticle drug delivery vehicles (e.g., lipid bilayer coated mesoporous silica nanoparticles (MSNP)⁴⁵⁻⁴⁸) for the delivery of the camptothecin analog irinotecan (IRIN) in an immunotheraputic context was investigated. In particular, it was sought to determine whether these carriers can be used for immunotherapy when administered in combination with one or more checkpoint inhibitors. As a prelude to animal studies, initial investigations were performed using KPC cells, derived from a spontaneous KraS^{LSL-G12D/+}; Trp53^{LSL-R172H/+}; Pax-1-Cre (KPC) tumor^{49,50}. It was surprisingly observed that in addition to its traditional chemotherapeutic effects, IRIN was capable of inducing an early impact on an acidifying cellular compartment (lysosome), where the neutralization of the acidic pH by the weak base properties of IRIN, could disrupt autophagosome fusion. We further discovered that interference in autophagy flux promotes the triggering of ER stress and expression of PD-L1 by the cancer cells, suggesting that anti-PD-1 therapy could possibly be used in synergy with an IRIN-silicasome.

This hypothesis was tested in an orthotopic KPC model, which demonstrated triggering of an immune response that could be augmented by anti-PD-1 antibodies. It was a particularly surprising discovery that combination therapy with silicasomes is significantly better than the combined use of free irinotecan or ONIVYDE® with anti-PD-1.

In view of these observations, it is believed that the IRIN-silicasome for chemo-immunotherapy in combination with blocking of the PD–1/PD-L1 axis can provide for an effective approach to the treatment of various cancers. Moreover, as described herein it is believed that the drug delivery vehicle need not be limited to a lipid bilayer coated mesoporous silica nanoparticles (MSNP), but additionally can comprise other porous nanoparticles coated with a lipid bilayer. Illustrative alternative drug delivery vehicles include but are not limited to, as described herein, porous inorganic nanoparticle, a metal-organic framework nanoparticle, or a porous organic nanoparticle.

It will also be recognized that the methods described herein need not be limited to the camptothecin analog irinotecan, but certain other camptothecin analogs can also be utilized. Additionally, various checkpoint inhibitors suitable for the methods described herein include but are not limited to PD1 inhibitors, PD-L1 inhibitors, and CTLA4 inhibitors. In certain embodiments, the drug delivery vehicles described herein can additionally contain one or more autophagy inhibitors. Accordingly, in certain embodiments a method of treating a cancer in a mammal is provided where the method comprises: administering to the mammal, or causing to be administered to the mammal, an effective amount of: i) one or more checkpoint inhibitor(s); and ii) one or more camptothecin analogs, and/or one or more autophagy inhibitors wherein the one or more camptothecin analog(s), and/or one or more autophagy inhibitors are provided inside a delivery vehicle where the delivery vehicle comprises: a nanoparticle comprising one or more cavities disposed within said nanoparticle and an outside surface where said one or more cavities are in fluid communication the outside surface of said nanoparticle; and the one or more camptothecin analog(s), and/or one or more autophagy inhibitors are disposed within the one or more cavities; and a lipid bilayer is disposed on the surface of said nanoparticle where said lipid bilayer fully encapsulates the nanoparticle.

In certain embodiments, the drug delivery vehicles containing the camptothecin analog(s) and the checkpoint inhibitors are administered simultaneously. In certain embodiments, the camptothecin analog(s) and the checkpoint inhibitors are administered as a combined formulation. In certain embodiments, the camptothecin analog(s) and the checkpoint inhibitors are administered as separate formulations. In certain embodiments, the drug delivery vehicles containing the camptothecin analog(s) and the checkpoint inhibitors are administered at different times.

The various camptothecin analogs, checkpoint inhibitors, drug delivery vehicles, and autophagy inhibitors are described further below.

### Camptothecin Analogs

In various embodiments, the methods described herein involve administering to a mammal an effective amount of one or more camptothecin analogs in combination with one or more checkpoint inhibitors and, optionally, one or more autophagy inhibitors. While proof of principle is demonstrated in Example 1 using the camptothecin analog irinotecan (IRIN), other suitable camptothecin analogs are known and will be available to one of skill in the art.

For example, numerous camptothecin analogs are described in U.S. Pat. Publication Nos: US2016/0115183, US2008/0261919, US2012/0136153, US2009/0198061, US2009/0099224, US2009/0099166, US2008/0269169, US2008/0027224, US2007/0259905, US2005/0209263, US2005/0096338, US2005/0014775, US2004/0266804, US2004/0266803, US2004/0258754, US2004/0029835, US2003/0105324, US2003/0088101, US2002/0193598, US2001/0003779, and the like, which are incorporated herein by reference for the camptothecin analogs described therein.

Illustrative camptothecin analogs (other than inrinotecan) include, but are not limited to belotecan (CKD-602), topotecan, silatecan (db-67, ar-67), cositecan (bnp-1350), exatecan, lurtotecan, gimatecan (st1481), rubitecan, homocamptothecin, trastuzumab deruxtecan, Rubitecan, Beltecan, Exatecan, Lurtotecan, Gimatecan, Diflomotecan, Karenitecan, Silatecan, Namitecan, ZBH-1205, Elomotecan, DRF-1042, Delimotecan, NSC606985, Chimmitecan, Genz-644282, non-CPT1 and the like. In certain embodiments, the analog comprises irinotecan. In certain embodiments, the analog comprises belotecan (CKD-602).

In various embodiments, the camptothecin analog is desirably a weak-basic analog. A weak base is a base that upon dissolution in water does not does dissociate completely, so that the resulting aqueous solution contains only a small proportion of hydroxide anions and the concerned basic radical, together with a large proportion of undissociated molecules of the base.

In certain embodiments, the analog comprises a water-soluble analog (e.g., an analog that has a solubility of greater than 5 mg/mL in water, or greater than 8 mg/mL in water, or greater than 10 mg/mL in water, or greater than about 12 mg/mL in water or greater than about 15 mg/mL in water, or greater than about 20 mg/mL in water, or greater than about 22 mg/mL in water).

The foregoing camptothecin analogs are illustrative and non-limiting. Using the teaching provided herein numerous other camptothecin analogs suitable for use in the methods described herein will be available to one of skill in the art.

### Checkpoint Inhibitors

In various embodiments, the methods described herein involve administering to a mammal an effective amount of one or more checkpoint inhibitors in combination with one or more camptothecin analogs and, optionally, one or more autophagy inhibitors.

The use of checkpoint inhibitors in the treatment of cancer is a form of cancer immunotherapy. The therapy immune checkpoints, key regulators of the immune system that when stimulated can dampen the immune response to an immunologic stimulus. Some cancers can protect themselves from attack by stimulating immune checkpoint targets. Checkpoint therapy can block inhibitory checkpoints, restoring immune system function.

Checkpoint inhibitors are known to those of skill in the art. In various embodiments, illustrative checkpoint inhibitors block the binding interactions of CTLA4, and/or PD-1, and/or PD-L1. PD-1 is the transmembrane “programmed cell death 1” protein (also called PDCD1 and CD279), which interacts with PD-L1 (PD-1 ligand 1, or CD274). PD-L1 on the cell surface binds to PD1 on an immune cell surface, which inhibits immune cell activity. Among PD-L1 functions is a key regulatory role on T cell activities. It appears that (cancer-mediated) upregulation of PD-L1 on the cell surface may inhibit T cells that might otherwise attack. Antibodies (or other inhibitors) that bind to either PD-1 or PD-L1 and therefore block the interaction may allow the T-cells to attack the tumor.

CTLA-4, also known as CD152, is a protein receptor that functions as an immune checkpoint and downregulates immune responses. CTLA4 is constitutively expressed in regulatory T cells but only upregulated in conventional T cells after activation -a phenomenon which is particularly notable in cancers.

Other illustrative, but non-limiting checkpoint inhibitors include, but are not limited to Tim-3, ICOS (see, e.g., Amatore et al. (2018) Expert. Opin. Ther. Targets, 22(4): 343-351), 4-1BB (see, e.g., Compte et al. (2018) Nat. Commun. 9(1): 4809), and OX-40 (see, e.g., Polesso & Moran (2019) Cancer Immunol. Res. 7(2): 269-281). Am

### PD-L1 Inhibitors

PD-L1 inhibitors are well known to those of skill in the art. Illustrative PD-L1 inhibitors include, but are not limited to Atezolizumab, Avelumab, Durvalumab, KN035, CK-301 by Checkpoint Therapeutics, AUNP12, CA-170, and BMS-986189, and the like. Atezolizumab (Tecentriq) is a fully humanised IgG1 (immunoglobulin 1) antibody developed by Roche Genentech. Avelumab (Bavencio) is a fully human IgG1 antibody developed by Merck Serono and Pfizer. Durvalumab (Imfinzi) is a fully human IgG1 antibody developed by AstraZeneca. KN035 is a PD-L1 antibody with subcutaneous formulation currently under clinical evaluations. CK-301 is an anti-PD-L1 antibody by Checkpoint Therapeutics.

PD-L1 inhibitors are not limited to antibodies. For example, AUNP12 is a 29-mer peptide, the first peptic PD-1/PD-L1 inhibitor developed by Aurigene and Laboratoires Pierre Fabre that is being evaluated in clinical trial, following promising in vitro results. CA-170, discovered by Aurigene/Curis as a PD-L1 and VISTA antagonist, was indicted as a potent small molecule inhibitor in vitro. BMS-986189 is a macrocyclic peptide PD-L1 inhibitor discovered by Bristol-Myers Squibb.

The foregoing PD-L1 inhibitors are illustrative and non-limiting. Using the teaching provided herein numerous other PD-L1 inhibitors suitable for use in the methods described herein will be available to one of skill in the art.

### PD-1 Inhibitors

PD-1 inhibitors are also well known to those of skill in the art. Such inhibitors include, but are not limited to Pembrolizumab, Nivolumab (Opdivo), Cemiplimab (Libtayo), Spartalizumab (PDR001), Camrelizumab (SHR1210)Sintilimab (IBI308), Tislelizumab (BGB-A317), Toripalimab (JS 001), Dostarlimab, INCMGA00012 (MGA012), AMP-224, AMP-514 (MEDI0680), and the like.

Pembrolizumab (formerly MK-3475 or lambrolizumab, Keytruda) was developed by Merck. Nivolumab (Opdivo) was developed by Bristol-Myers Squibb. Cemiplimab (Libtayo) was developed by Regeneron Pharmaceuticals. Spartalizumab (PDR001) is a PD-1 inhibitor developed by Novartis. Camrelizumab (SHR1210) is an anti-PD-1 monoclonal antibody introduced by Jiangsu HengRui Medicine Co., Ltd. Sintilimab (IBI308), a human anti-PD-1 antibody developed by Innovent and Eli Lilly. Tislelizumab (BGB-A317) is a humanized IgG4 anti-PD-1 monoclonal antibody. Toripalimab (JS 001) is a humanized IgG4 monoclonal antibody against PD-1. Dostarlimab (TSR-042, WBP-285) is a humanized monoclonal antibody against PD-1. INCMGA00012 (MGA012) is a humanized IgG4 monoclonal antibody developed by Incyte and MacroGenics. AMP-224 by AstraZeneca/MedImmune and GlaxoSmithKline[22] is PD-1/B7 Fc fusion protein. AMP-514 (MEDI0680), by AstraZeneca, is a monoclonal antibody that consists of a humanized immunoglobulin gamma 4 kappa (IgG4k) specific to programmed cell death-1 (PD-1) protein.

The foregoing PD1 inhibitors are illustrative and non-limiting. Using the teaching provided herein numerous other PD1 inhibitors suitable for use in the methods described herein will be available to one of skill in the art.

### CTLA-4 Inhibitors

CTLA-4 inhibitors are well known to those of skill in the art. A number of anti-CTLA4 antibodies are described for example in U.S. Pat. Publication Nos: US2020/0206346, US 2019/0276542, US 2019/0241662, US 2019/0225690, US 2019/0185569, US 2019/0177414, US2017/0216433, US 2013/0142805, US 2012/0135001, US 2012/0121604, US 2010/0278828, US 2010/0098701, US 2009/0252741, US 2009/0074787, and US 2008/0152655, which are incorporated herein by reference for the anti-CTLA4 antibodies described therein.

Additionally, the monoclonal antibody Ipilimumab has been approved by the FDA for clinical use.

The foregoing CTLA4 inhibitors are illustrative and non-limiting. Using the teaching provided herein numerous other CTLA4 inhibitors suitable for use in the methods described herein will be available to one of skill in the art.

A number of checkpoint inhibitors are approved for clinical use in the United states by the Food and Drug administration. A list of illustrative FDA approved checkpoint inhibitors is provided in Table 1.

TABLE 1

FDA approved checkpoint inhibitors

| Name | Target | Approved |
|---|---|---|
| Ipilimumab | CTLA-4 | 2011 |
| Nivolumab | PD-1 | 2014 |
| Pembrolizumab | PD-1 | 2014 |
| Atezolizumab | PD-L1 | 2016 |
| Avelumab | PD-L1 | 2017 |
| Durvalumab | PD-L1 | 2017 |
| Cemiplimab | PD-1 | 2018 |

### Bispecific Inhibitors

In certain embodiments, the checkpoint inhibitors comprise bispecific moieties (e.g., bispecific antibodies or antibodies attached to a moiety that binds to a receptor ligand). In certain embodiments, the bispecific moieties comprise an antibody that binds to PD1, or to PD-L1, or to CTLA4 attached to another binding moiety. In certain embodiments, the bispecific moiety comprise an anti-PD1 antibody attached to a cytokine, a costimulatory molecule, or a dominant negative receptor with PD1/PD-L1 full antagonistic activity. In certain embodiments, the bispecific moiety comprises an anti-PD1 antibody attached to a cytokine (e.g., IL-7). In certain embodiments, the bispecific moiety comprises an anti-PD1 antibody attached to an antibody that binds to CTLA4.

The foregoing checkpoint inhibitors are illustrative and non-limiting. Using the teachings provided herein, numerous other checkpoint inhibitors will be available to one of skill in the art for use in the methods described herein.

### Autophagy Inhibitors

In various embodiments, the methods described herein involve administering to a mammal an effective amount of one or more checkpoint inhibitors in combination with one or more camptothecin analogs and, optionally, one or more autophagy inhibitors. Autophagy inhibitors are well known to those of skill in the art and can readily be incorporated into the drug delivery vehicles described herein, or in certain embodiments incorporated into the lipid bilayer.

Autophagy inhibitors are well known to those of skill in the art. Illustrative autophagy inhibitors include, but are not limited to chloroquine, hydroxychloroquine, a member of the bafilomycin family, and the like. In certain embodiments, the autophagy inhibitor comprises chloroquine. In certain embodiments, the autophagy inhibitor comprises hydroxychloroquine. In certain embodiments, the autophagy inhibitor comprises a member of the bafilomycin family (e.g., bafilomycin A1, bafilomycin B1, bafilomycin B2, bafilomycin C1, bafilomycin C2, bafilomycin C1 amide, bafilomycin C2 amide, 9-hydroxybafilomycin D, 29-hydroxybafilomycin D, bafilomycin D, and bafilomycin E, and the like).

Other illustrative, but non-limiting examples of autophagy inhibitors are shown in Table .

| Name | Structure | Biochemical/physiological Actions |
|---|---|---|
| N-Acetyl-L-cysteine | 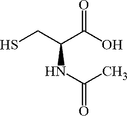 | Antioxidant and mucolytic agent. Increases cellular pools of free radical scavengers. |
| ARN5187 -Calbiochem | 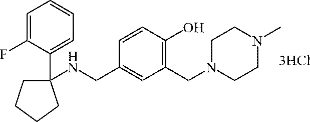 | Cell permeable, Primary Target = REV-ERBβ |
| L-Asparagine | 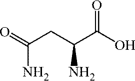 | |
| Autophagy Inhibitor, 3-MA - CAS 5142-23-4 - Calbiochem | 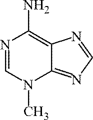 | Autophagic sequestration blocker. Acts as an inhibitor of III PI3-Kinase. |
| DBeQ | 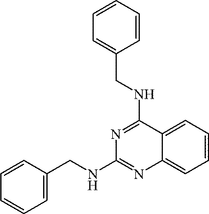 [SMILES: c1ccc(CNc2nc(NCc3ccccc3)c3ccccc3n2)cc1] | DBeQ is a potent and specific inhibitor of ATPase p97. |
| E-64d protease inhibitor | 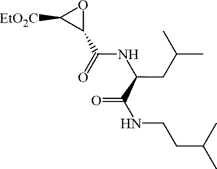 [SMILES: CCOC(=O)[C@H]1O[C@@H]1C(=O)N[C@@H](CC(C)C)C(=O)NCCC(C)C] | E-64d is an epoxysuccinyl peptide and an inhibitor of cysteine protease cathepsin B, calpains 1 and 2. |
| GMX1778 | 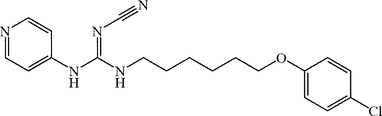 [SMILES: N#C/N=C(\NCCCCCCOc1ccc(Cl)cc1)Nc1ccncc1] | GMX1778 (CHS-828) is a competitive inhibitor of nicotinamide phosphoribosyltransferase (NAMPT). |
| LY-294,002 hydrochloride | 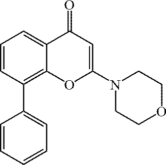 [SMILES: O=c1cc(N2CCOCC2)oc2c(-c3ccccc3)cccc12] | Specific cell permeable phosphatidylinositol 3-kinase inhibitor. |
| 3-Methyladenine autophagy inhibitor | 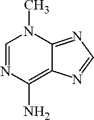 | |
| MHY1485 | 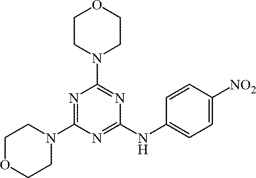 [SMILES: O=[N+]([O-])c1ccc(Nc2nc(N3CCOCC3)nc(N3CCOCC3)n2)cc1] | MHY1485 binds to the mammalian target of rapamycin (mTOR) and stimulates its action. |
| mTOR Activator, MHY1485 -Calbiochem | 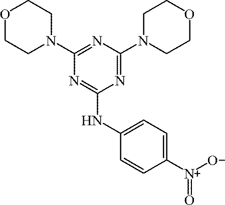 [SMILES: O=[N+]([O-])c1ccc(Nc2nc(N3CCOCC3)nc(N3CCOCC3)n2)cc1] | |
| Pepstatin A microbial, ≥90% (HPLC) | 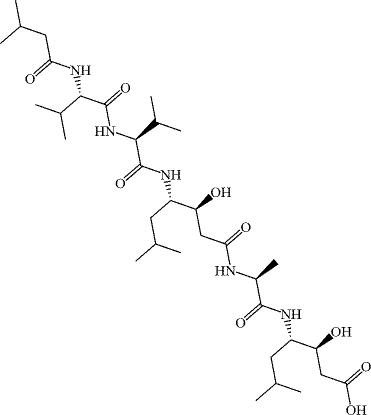 [SMILES: CC(C)CC(=O)N[C@H](C(=O)N[C@H](C(=O)N[C@@H](CC(C)C)[C@@H](O)CC(=O)N[C@@H](C)C(=O)N[C@@H](CC(C)C)[C@@H](O)CC(=O)O)C(C)C)C(C)C] | Pepstatin A is an inhibitor of acid proteases (aspartyl peptidases). |
| SBI-0206965 | 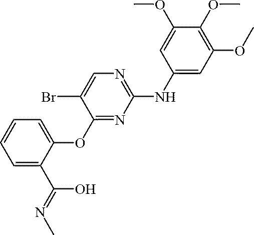 [SMILES: C/N=C(\O)c1ccccc1Oc1nc(Nc2cc(OC)c(OC)c(OC)c2)ncc1Br] | |
| Spautin-1 | 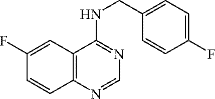 [SMILES: Fc1ccc(CNc2ncnc3ccc(F)cc23)cc1] | Spautin-1 inhibits the activity of two ubiquitin-specific peptidases, USP10 and USP13. |
| Thapsigargin | 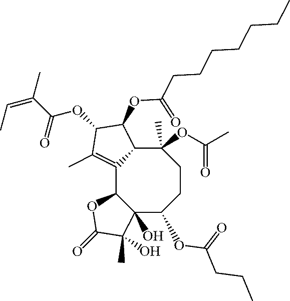 [SMILES: C/C=C(/C)C(=O)O[C@H]1C(C)=C2[C@H]([C@@H]1OC(=O)CCCCCCC)[C@@](C)(OC(C)=O)CC[C@H](OC(=O)CCC)[C@@]1(O)[C@H]2OC(=O)[C@@]1(C)O] | Sarco-endoplasmic reticulum Ca2+-ATPases inhibitor |
| Wortmanin | 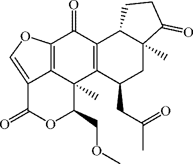 [SMILES: COC[C@H]1OC(=O)c2coc3c2[C@@]1(C)C1=C(C3=O)[C@@H]2CCC(=O)[C@@]2(C)C[C@H]1CC(C)=O] | Wortmannin is a potent and specific phosphatidylinositol 3-kinase (PI3-K) inhibitor. |

In certain embodiments, the autophagy inhibitor comprises a nanoparticle and such nanoparticles can readily be incorporated into the nanoparticle comprising the drug delivery vehicles described herein. Without being bound to a particular theory, it is believed that nanoparticle can prevent diffusion of lysosomes with the autophagosome by a mechanism that involves sequestration of cellular phosphates (which interferes in the small motor proteins that fuse these compartments). Illustrative nanoparticles include but are not limited to nanoparticles comprising a metal or metal oxide, a rare earth or rare earth oxide, or silica. In certain embodiments, the autophagy-inhibiting nanoparticle comprises a metal (e.g., gold, silver, iron, copper, titanium, aluminum, and the like). In certain embodiments, the autophagy-inhibiting nanoparticle comprises a metal oxide (e.g., zinc oxide, iron oxide, iron oxide/gold, copper oxide, titanium dioxide, and ferroferic oxide, and the like). In certain embodiments, In certain embodiments, the autophagy-inhibiting nanoparticle comprises a rare earth or rare earth oxide (e.g., a rare earth such as scandium, yttrium, lanthanum, cerium, praseodymium, neodymium, promethium, samarium, europium, gadolinium, terbium, dysprosium, holmium, erbium, thulium, ytterbium, and lutetium). In certain embodiments, In certain embodiments, the autophagy-inhibiting nanoparticle comprises a rare earth oxide (e.g., cerium oxide, europium hydroxide, and the like).

In certain embodiments, the autophagy inhibitor comprises a nanoparticle. Without being bound to a particular theory, it is believed that nanoparticle can prevent diffusion of lysosomes with the autophagosome by a mechanism that involves sequestration of cellular phosphates (which interferes in the small motor proteins that fuse these compartments).

These autophagy-inhibiting nanoparticles are illustrative and non-limiting. Using the aching provided herein, numerous other autophagy-inhibiting nanoparticles will be available to one of skill in the art.

In general, the foregoing autophagy inhibitors are illustrative and non-limiting. Using the teachings provided herein, numerous other autophagy inhibitors will be available to one of skill in the art for use in the methods described herein.

### Nanoparticles

In various embodiments the drug delivery vehicles described herein comprise a nanoparticle containing one or more cavities where the nanoparticle is disposed within and fully encapsulated by a lipid bilayer. The camptothecin analog(s) and, in certain embodiments, the autophagy inhibitors when present, are disposed within the cavities. These nanoparticles include, but are not limited to a porous inorganic nanoparticle, a porous organic nanoparticle, or a metal-organic framework nanoparticle. When the nanoparticle comprise a mesoporous silica nanoparticle the drug delivery can be referred to as a “silicasome”.

### Porous Inorganic Nanoparticles

Porous inorganic nanoparticles include, but are not limited to porous calcium carbonate nanoparticles, porous calcium phosphate nanoparticles and porous silica nanoparticles. In certain embodiments the porous inorganic nanoparticle comprises a single cavity. In certain embodiments this cavity is simply a channel into the nanoparticles. In certain embodiments the cavity comprises a hollow interior of the nanoparticle with a single aperture (pore) penetrating through to the surface of the nanoparticle. In certain embodiments the single-cavity nanoparticle comprises a nanobowl (i.e., a bowel shaped nanoparticle (concave nanostructures with an opening)). Single cavity nanoparticles may be fabricated by any of a number of methods well known to those of skill in the art. By way of example, lipid bilayer covered nanobowls are described by Chen et al. (2020) Nano Letters, DOI: 10.1021/acs.nanolett.0c00495.

In certain embodiments the porous nanoparticle comprises a porous silica nanoparticle. In certain embodiments the porous silica nanoparticle comprises a mesoporous silica nanoparticle (MSN), a mesoporous organosilica nanoparticle (MON), and/or a periodic mesoporous organosilica (PMO) nanoparticle.

MSNs, MONs, and PMOs are commonly fabricated using sol-gel processes in aqueous solutions (Croissant et al. (2015) Nanoscale, 7: 20318-20334; Wu et al. (2013) Chem. Soc. Rev. 42: 3862-3875; Yano & Fukushima (2004) J. Mater. Chem. 14: 1579-1584; Nakamura et al. (2007) J. Phys. Chem. C, 111: 1093-1100). The conventional sol-gel synthesis has been studied extensively and allows precise control of nanoparticle properties such as size, pore size and geometry, particle modification, and/or surface functionalization (see, e.g., Wu et al. (2013) Chem. Soc. Rev. 42: 3862-3875). In one illustrative sol-gel synthesis, silica particles are formed via hydrolysis of various silanes and/or silicates with a subsequent silica condensation:

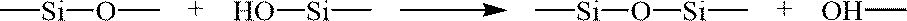

In one illustrative, but non-limiting embodiment, synthesis takes place in an aqueous solution and can involve alcohol and ammonia or other catalyst (see, e.g., Yano & Fukushima (2004) J. Mater. Chem. 14: 1579-1584). The synthesis reaction speed depends on the pH value with the maximum silica condensation rate at normal pH conditions. Types and concentrations of the used reagents affect the resulting particle size. Tetraethyl orthosilicate (TEOS), tetramethyl orthosilicate (TMOS) and other compounds can be used as silicon sources. Using TMOS facilitates the generation of monodispersed particles since it reacts preferentially with the silanol groups on the surface of already formed particles rather than with itself to create the new particles (see, e.g., Nakamura et al. (2007) J. Phys. Chem. C, 111: 1093-1100). To inhibit silica growth and, thus, obtain smaller MSNs, surface-protection agents can be used, such as triethanolamine (TEA), poly (ethylene glycol) (PEG) and/or a second nonionic surfactant (see, e.g., Möller et al. (2007) Adv. Funct. Mater. 17: 605-612). These agents can also be useful for isolation of the growing silica particles from each other, preventing their aggregation and the growth of silica bridges between neighboring particles.

In certain embodiments, to obtain MSNs, micelles can be used as a soft template to form the mesoporous structure. In one illustrative, but non-limiting embodiment, the silica particles are grown on the templates as starting points for the condensation. Surfactants such as cetyltrimethylammonium bromide (CTAB) can be added to the solution as well. At low concentrations just above the critical micellar concentration, the surfactant molecules bind together and form small spherical micelles. At higher concentrations, micelles can have cylindrical or other shapes. These micelles are positively charged and attract negatively charged silanes, facilitating their condensation. Addition of the second surfactant can lead to the formation of the more complicated micellar structures, allowing further modification of the MSNs pore structure. Similar to the micelles, vesicles can be used as templates for the MSN growth (see, e.g., Yeh et al. (2006) Langmuir, 22: 6-9). In certain embodiments, inorganic nanoparticles, such as metal (Au, Pt) or metal oxide (Fe₃O₄) nanoparticles could be incorporated into the structure of MSNs as desired (see, e.g., Kneževi’ et al. (2013) RSC Adv. 3: 9584-9593; Timin et al. (2016) Mater. Chem. Phys. 183: 422-429; Ott et al. (2015) Chem. Mater. 2015, 27: 1929-1942). They can be used as the templates for the MSNs growth as well. Such “hybrid” nanoparticles can be capable of both carrying a drug load and acting as contrast agents for bio-imaging. In certain embodiments to produce larger pore sizes to accommodate higher quantities of molecules or simply larger molecules (e.g., biomolecules, such as DNA and proteins a swelling agent can be utilized. Several swelling agents can be used to increase the pore sizes, e.g., trimethylbenzene (TMB) (see, e.g., Zhang et al. (2011) J. Colloid Interface Sci. 361: 16-24). Another way to increase the size of the pores is the use of the block-polymers as templates (see, e.g., Han & Ying (2005) Angew. Chem. 117: 292-296).

In certain embodiments a so-called “liquid calcination” method using high boiling solvents can be used to retain liquid phase during calcination. Silanol groups can also be removed from MSN surface via a silane ethanol solution, reducing the bridging. The calcination process can be avoided entirely if templating is done using a thermosensitive polymer (poly (N-isopropylacrylamide)), which forms aggregates at higher temperatures and dissolves at lower temperatures (see, e.g., Du et al. (2009) Langmuir, 25: 12367-12373).

The mixture of silane [usually tetraethyl orthosilicate(TEOS)] and an organosilane induces the formation of MONs and PMO. In this case, in certain embodiments, the surfactant templates can be removed with less aggressive extraction procedures, in order not to destroy the inorganic-organic framework of MONs and PMO. In certain embodiments, the calcination procedure, which can be used for MSNs, may not be completely appropriate for MONs and PMO. In general, harsh pH and temperature conditions are usually employed for the extracting process. The silica-etching chemistry [alkaline or hydrofluoric acid HF) etching] can be introduced into the synthesis to form the hollow PMO structure (see e.g., Chen et al. (2013) Adv. Mater. 25: 3100-3105). For this, the PMO layer can be directly deposited onto the surface of silica particles in order to form well-defined solid silica core/PMO shell.

The chemical stability of some families of PMOs is higher than for the silica particles under etching. Therefore, the silica core can be selectively removed under alkaline or HF etching producing hollow periodic mesoporous structure. Illustrative, but non-liming examples of fabrication protocols are described by Wu et al. (2013) Chem. Soc. Rev. 42: 3862-3875 and by Chen et al. (2014) J. Am. Chem. Soc. 136: 16326-16334.

Uniform mesoporous silica particles of different diameters can be prepared using various synthetic conditions (e.g., controlling pH values or time of reaction). For instance, a simple method for tailoring the size of well-ordered and dispersed MSNs by adjusting the pH of the reaction medium, which leads to the series of MSNs with diameter sizes ranging from 30 to 280 nm is described by Lu et al. (2009) Small, 5: 1408-1413. It also possible to control particle growth at different times of the reaction. Smaller particles (140 nm) emerged for 160 s into the reaction process grew to their final size (500 nm) in 600 s.

In one illustrative, but non-limiting embodiment, mesoporous silica nanoparticles (MSNPs) are synthesized as a large batch, as previously described by Liu et al. (2019) ACS Nano. 13(1): 38-53. As illustrated herein in Example 1, this involves the addition of 0.9 L of 25 wt% CTAC in water to 17.1 L pure water in a beaker, stirred at 85° C. 72 g triethanolamine is added, followed by 600 mL TEOS. After stirring for 4 hours and cooling to room temperature, the bare MSNPs are precipitated with ethanol and CTAC is removed by washing in acidic ethanol, with sonication. MSNPs at 80 mg/mL in ethanol are centrifuged at 21,000 x g for 15 minutes to pellet the nanoparticles. After removal of the ethanol supernatant, the MSNP pellet is resuspended in 123 mM ammonium sulfate in water by bath sonication.

Potential bioaccumulation is one of the biggest limitations for silica nanodrug delivery systems in cancer. Accordingly, in certain embodiments, the porous silica nanoparticles described herein (e.g., mesoporous silica nanoparticles) are modified to improve degradation and clearance. In one illustrative, but non-limiting example, the nanoparticles comprise a mesoporous silica /hydroxyapatite (MSNs/HAP) hybrid drug carrier, that provides enhanced biodegradability of silica. Synthesis of such nanoparticles is described by Hao et al. (2015) ACS Nano, 9(10): 9614-9625.

Other approaches to improving silica nanoparticle degradation include, but are not limited to noncovalent organic doping of silica, covalent incorporation of either hydrolytically stable or redox- and enzymatically cleavable silsesquioxanes, as well as bridged silsesquioxane (BS), and periodic mesoporous organosilica (PMO) NPs. Inorganically doped silica particles such as calcium-, iron-, manganese-, and zirconium-doped NPs, can also be used (see, e.g., Croissant et al. (2017) Adv. Mater., 29: 1604634).

In certain embodiments the mesoporous silica nanoparticles can be imine-doped silica nanoparticles. These nanoparticles contain imine groups embedded within the silica framework (see, e.g., Travaglini et al. (2019) Mater. Chem. Front., 3: 111-119). These methods of increasing degradability of silica nanoparticles are illustrative and non-limiting. Using the teaching provided herein, numerous other porous silica nanoparticles modified for enhanced biodegradation will be available to one of skill in the art.

Illustrative mesoporous silica nanoparticles include, but are not limited to MCM-41, MCM-48, and SBA-15 (see, e.g., Katiyaret al. (2006) J. Chromatog. 1122(1-2): 13-20).

In certain embodiments the porous inorganic nanoparticle comprises a porous calcium carbonate nanoparticle or a porous calcium phosphate nanoparticle. Means for fabricating calcium carbonate or calcium phosphate nanoparticles are known to those of skill in the art (see, e.g., Oiso & Yamanaka (2018) Adv. Powder Technol., 29(3): 606-610; Trofimov et al. (2108) Pharmaceutics, 10: 167).

Methods for obtaining porous CaCo3 nanoparticles include, but are not limited to chemical methods (see, e.g., Trushina (2014) Mater. Sci. Eng. C, 45: 644-658; Svenskaya et al. (2016) Adv. Powder Technol. 27: 618-624; Parakhonskiy et al. (2015) J. Nanobiotechnol. 13: 53. doi: 10.1186/s12951-015-0111-7; Salomão et al. (2017) Adv. Tissue Eng. Regen. Med. eISSN: 2572-8490), microbiological methods (see, e.g., Wang et al. (2010) J. Phys. Chem. B, 114: 5301-5308; Achal & Pan (2014) Appl. Biochem. Biotechnol. 173: 307-317; Chekroun et al. (2004) J. Sediment. Res. 74: 868-876; Rodriguez-Navarro et al. (2007) Geochim. Cosmochim. Acta. 71: 1197-1213). The latter approach involves a bacteria-mediated synthesis, employing products of microbial metabolism containing carbonate ions that react with the calcium ions present in the environment to form CaCO₃ (see, e.g., Salomão et al. (2017) Adv. Tissue Eng. Regen. Med. eISSN: 2572-8490). Chemical methods generally utilize an emulsion technique (see, e.g., Fujiwara et al. (2010) Cryst. Growth Des. 10: 4030-4037; Maleki Dizaj et al. (2015) Expert Opin. Drug Deliv. 12: 1649-1660) and a precipitation reaction (see, e.g., Shirsath et al. (2017) Ultrason. Sonochem. 35: 124-133). One conventional approach for industrial production of CaCO₃ nanoparticles is precipitation by carbon dioxide bubbling through a calcium containing solution (gas diffusion method) (see, e.g., Shirsath et al. (2017) Ultrason. Sonochem. 35: 124-133; Shirsath et al. (2015) Ultrason. Sonochem. 24: 132-139). Other chemical methods include, but are not limited to a controlled double-jet precipitation technique (see, e.g., Som et al. (2016) Nanoscale, 8: 12639-12647; Jiang et al. (2009) Chem. Commun. 5853-5855; Stavek et al. (1990) Mater. Lett. 9: 90-95) and a solvothermal growth method (see, e.g., Li et al. (2002) J. Cryst. Growth, 236: 357-362).

Similarly, there are many different methods to produce porous calcium phosphate nanoparticles with different morphologies and sizes. One illustrative synthesis method of porous calcium phosphate nanosized materials involves mixing of water-soluble salts of calcium and phosphate. With this technique, it is possible to control size, shape and crystallinity of particles by changing conditions of the precipitation reaction. For example, to obtain porous calcium phosphate spherical nanoparticles microwave assisted hydrothermal method is often used. This method can involve adenosine 5-triphosphate disodium salt (ATP) as the phosphorus source and stabilizer (see, e.g., Qi et al. (2013) Chemistry, 19: 981-987). As one illustrative, but non-limiting example, porous calcium phosphate particles with an average diameter of 260 nm have been synthesized by mixing calcium chloride dehydrate with ATP and further microwave treatment (Id.). The resulted nanoparticles showed good stability in aqueous solutions at different pH for more than 150 h. Alternatively, porous calcium phosphate nanoparticles with different morphology and crystallinity can be prepared by changing the ratio of precursors.

Porous calcium phosphate nanoparticles can also be synthesized by aging a mixture of calcium hydroxide and sodium triphosphate in the presence of hydrochloric acid (see, e.g., Kandori et al. (2010) J. Phys. Chem. C, 114: 6440-6445). To obtain differently shaped particles, the aging temperatures can be varied from, e.g., 100 to 150° C., as well as amount of precursors in the reaction (Id.). Additionally, hollow calcium phosphate nanoparticles can be obtained with a templating method (see, e.g., Ding et al. (2015) J. Mater. Chem. B, 3: 1823-1830). Different materials can be employed as templates. These include, for example, soybean lecithin (Id.), block copolymer micelles (see, e.g., Bastakoti et al. (2012) Chem. Commun. 48: 6532-6534). As one illustrative, but non-limiting example, an anionic triblock copolymer (poly (styrene-acrylic acid-ethylene glycol) has been used to form calcium phosphate hollow particles (see, e.g., Yeo et al. (2012) Ceram. Int. 38: 561-570). This type of template helps to overcome the problem of crystal overgrowth and allows the formation of nanosized hollow particles around 30 nm. Liposomes can also be used as templates to form hollow calcium phosphate particles (see, e.g., Yeo et al. (2012) Ceram. Int. 38: 561-570; Schmidt et al. (2004) Chem. Mater. 16: 4942-4947; Schmidt & Ostafin (2002) Adv. Mater. 14: 532-535). For example, 1,2-dioleoyl-sn-glycero-3 phosphate sodium salt (DOPA) and 1,2-dipalmitoyl-sn-glycero-3-phosphate sodium salt (DPPA) has been used as templates due to their negative charged head group, which can help the deposition of calcium and phosphate ions around the liposomes (see, e.g., Yeo et al. (2012) Ceram. Int. 38: 561-570). Resulting hollow nanoparticles were 64 and 104 nm, respectively (Id.). Polymer complexes can be also employed as templates to form hollow calcium phosphate particles (see, e.g., Zhang et al. (2009) Biomed. Mater. 4: 031002). Hollow calcium phosphate microspheres have been prepared using chitosan-polyacrylic acid (CS-PAA) as the template (Id.). The formation mechanism of the hollow structure was based on the electrostatic interactions between chitosan (CS) and poly acrylic acid (PAA). The size of CS-PAA spheres could be adjusted by changing the ratio and concentration of CS and PAA in the reaction (Id.).

The foregoing nanoparticles are illustrative and non-limiting. Using the teachings provided herein, numerous other porous inorganic nanoparticles will be available to one of skill in the art.

### Porous Organic Nanoparticles

In certain embodiments the nanoparticles comprise a porous organic material. Illustrative porous organic materials include but are not limited to porous biocompatible polymers. Such porous biocompatible polymers are well known to those of skill in the art. Thus, for example, U.S. Pat. No: 10,549,014 describes the synthesis of porous polymers of the polyaryletherketone (PAEK) family (e.g., polyether ether ketone (PEEK), carbon reinforced PEEK, polyether ketone ketone (PEKK), PEKEKK (polyetherketoneetherketoneketone), polyaryletherketone (PAEK), polyetherketone (PEK), Polyetherketone Etherketone Ketone (PEKEKK), and the like), polycaprolactone (PCL), polylactic acid (PLA), polyglycolic acid (PGA), polyphenylene, self-reinforced polyphenylene, polyphenylsulphone, polysulphone, polyethylene terephthalate (PET), polyethylene, polyurethane, or a mixture thereof, and the like. Other porous biocompatible polymers include, for example, polymers include oligocarbonatedimethacrylate (OCM-2) porous polymer (see, e.g., Yudin et al. (2020) Polymer, 192: 122302 doi.org/10.1016/j.polymer.2020.122302), carbonate- and phthalate-containing dimethacrylates (see, e.g., Kovylin et al. (2019) Chem. Select, 4(14): 4147-4155), and the like.

In certain embodiments porous nanoparticles comprising the drug deliver vehicles described herein are formed from hydrogels. In this regard it is noted that the term “lipobeads” has been used to name spherical bipartite structures made of a hydrogel core coated with a lipid bilayer (see, e.g., Rahni & Kazarov (2017) Gels, doi.org/10.3390/gels3010007). In certain embodiments the hydrogel comprising the “nanoparticle” can comprise a hydrogel formed from one or more materials selected from the group consisting of poly(N-isopropylacrylamide) (PNIPA), poly(N-isopropylacrylamide-co-1-vinylimidazole) (PNIPA-VI), poly(acrylamide) (PAAm), poly(acrylamide), poly(N,N-dimethylacrylamide), poly(N,N-diethylacrylamide), poly(1-vinylimidazole), poly(sodium acrylate), poly(sodium methacrylate), poly(2-hydroxyethylmethacrylate) (HEMA), poly(N,N-dimethylaminoethyl methacrylate) (DMAEMA), poly(N-[tris(hydroxymethyl)methyl]acrylamide), poly(1-(3-methacryloxy)propylsulfonic acid) (sodium salt), poly(allylamine), poly(N-acryloxysuccinimide), poly(N-vinylcaprolactam), poly(1-vinyl-2-pyrrolidone), poly(2-acrylamido-2-methyl-1-propanesulfonic acid) (sodium salt), poly((3-acrylamidopropyl) trimethylammonium chloride), poly(diallyldimethylammonium chloride), and the like.

Methods of forming lipobeads comprising these and other hydrogels are described in U.S. Pat. Pubs: US 2003/0035842 A1, US 2010/0062054 A1, US 2013/0202667 A1, and the like as well as Rahni & Kazarov (2017) supra.

Using the teachings provided herein, nanoparticles comprising the porous polymers described above, and/or other porous polymers are readily available to those of skill in the art and, using the teaching described herein, can be used in the fabrication of the drug delivery vehicles described herein.

### Metal-Organic Framework Nanoparticles

In certain embodiments the nanoparticle comprise a metal organic framework (MOF) nanoparticle. Metal organic frameworks (MOFs) are a class of compounds consisting of metal ions or clusters coordinated to organic ligands to form one-, two-, or three-dimensional structures. They are a subclass of coordination polymers, with the special feature that they are often porous.

In various embodiments the MOF comprises metal ions or metal clusters and organic molecule linkers. In certain embodiments any metal ion can be used for the preparation of a MOF. In certain embodiments, the metal ion is selected from the group consisting of Zn, Cu, Ni, AI, Co, Fe, Mn, Cr, Cd, Mg, Ca, Zr, Gd, Eu, Tb, and mixtures thereof. In other embodiments, the metal ion is selected from the group consisting of Zn, Cu, Fe, Gd, Al, Mg, and mixtures thereof. In one embodiment, the metal ion is Zn. In another embodiment, the metal ion is Fe. In yet another embodiment, the metal ion is Cu. In yet another embodiment, the metal ion is Al.

In certain embodiments the metal-organic framework (MOF) may be a transition metal-based metal-organic framework (MOF). For example, the metal-organic framework (MOF) may be a zinc-based metal-organic framework (MOF), a cobalt-based metal-organic framework (MOF), a zirconium-based metal-organic framework (MOF), a chromium-based metal-organic framework (MOF), or other transition metal-based metal-organic frameworks (MOF).

In certain embodiments, the MOF is selected from the group consisting of zeolitic imidazolate frameworks (ZIFs), Universitetet i Oslo (University of Oslo) (UiOs), and (Materials of Institut Lavoisier frameworks (MILs). In other embodiments, the MOF is selected from the group consisting of ZIF-8, ZIF-67, ZIF-90, Fe-BTC, HKUST-1, and MIL-53, MIL-89, MIL-88A, MIL-100, UiO-66, UiO-66-NH2, MOF-801, MOF-804, Fe-NDC-M, and Fe-NDC-O MOFs. In certain embodiments the MOF is MIL-88A. In certain embodiments the MOF is ZIF-8. Methods of synthesizing such MOFs are described in U.S. Pat. Publication Nos: US 2017/0232420 A1, and US 2019/0247502 A1 which are incorporated herein by reference for the MOFs and synthesis methods described therein.

Other suitable MOFs include but are not limited to MOF-1201 (Ca¹⁴(L-lactate)₂₀(Acetate)₈(C₂H₅OH)(H₂O)] and MOF-1203 [Ca6(L-lactate)₃(Acetate)₉(H₂O)], based on Ca²⁺ ions and innocuous lactate and acetate linkers (see, e.g., U.S. Pat. Pub. No: US 2020/0095264 A1 which is incorporated herein by reference for the MOFs and synthesis methods described therein.

The MOFs can be synthesized by any of a number of methoeds well known to those of skill in the art. By way of illustrative, but non-limiting example, In certain embodiments MOFs may be synthesized from a metal salts/metal ions and organic ligands. In some embodiments, the organic ligands may be any suitable mono-, di-, tri-, or tetravalent ligands. The metal salt/metal ion may be of any suitable metal, such as a transition metal, for example: Iron (Fe), Titanium (Ti), or zirconium (Zr). In some embodiments, MOFs, including those based on Fe-NDC-M and Fe-NDC-O, may be synthesized using iron nitrate nonahydrate (Fe(NO₃)₃9H₂O), and 2,6 naphthalenedicarboxylic acid (2,6-NDC). In some embodiments, iron nitrate nonahydrate and 2,6-NDC may be reacted in the molar ratio of 10-1:10, 5:1-1:5, 3:1-1:3. 2:1-1:2, 1.5:1-1:1.5, or 1:1. Iron nitrate nonahydrate and 2,6 NDC may be reacted in a solvent. In some embodiments, the solvent may be dimethylformamide (DMF), dimethylacetamide (DMAC) or dimethylsulfoxide (DMSO). Iron nitrate nonahydrate and 2,6 NDC may be stirred in the solvent.

Then the mixture may be reacted. In some embodiments, the mixture may be subject to microwave irradiation. For example, the mixture may be heated in a microwave oven. The mixture may be subject to microwave irradiation of about 10 W-500 W, 10 W-300 W, 10 W-300 W, 20 W-250 W, 30 W-250 W, 50 W-250 W, 100 W-200 W, or 150 W-200 W. The mixture may be irradiated for 30 sec or longer, 1 min or longer, 2 min or longer, 3 min or longer, 5 min or longer, or 10 min or longer.

In some embodiments, the mixture may be heated instead of or in addition to microwave irradiation. For example, the mixture may be heated in an oven, such as a conventional electrical oven. In some embodiments, the mixture may be heated at 50° C.-200° C., 50° C.-170° C., 70° C.-170° C., 70° C.-150° C., 70° C.-130° C., 80° C.-120° C., or 90° C.-110° C. In some embodiments, the mixture may be heated for 3 hours or longer, 5 hour or longer, 10 hours or longer, 15 hours or longer, 20 hours or longer, or 24 hours or longer. The product may be separated from the reaction mixture, for example by centrifuge, washing and/or drying.

In one specific embodiment MIL-88A MOFs can be synthesized according to the protocol described by Illes et al. (2017) Chem. Mater. 29(19): 8042-8046. As described therein, MIL-88A MOFs are synthesized in a microwave assisted approach. In this synthesis route an aqueous solution of FeCl₃ • 6H₂O (1.084 g, 4.01 mmol) and fumaric acid (485 mg, 4.18 mmol) are given to water (20 ml, Milli-Q). The reaction mixture is stirred until the metal salt is completely dissolved. The reaction mixture was then given into a Teflon tube (80 ml) and placed into a microwave oven (e.g., Synthos 3000, Anton-Paar ) along with 3 additional vessels. Two of these vessels are filled with water (20 ml), the third vessel is filled with an aqueous FeCl₃ (20 ml, 1.084 g, 4.01 mmol) and is used to monitor the reaction progress. The vessels were heated under stirring with the sequence comprising heating 30 seconds to 80° C., holding at 80° C. for 5 min, followed by cooling over 45 min to room temperature.

In another illustrative, but non-limiting embodiment, MOF hollow spheres with controlled size in the 35-2000 µm range including MIL-88A frameworks, as well as various functional nanoparticles (silica, cobalt, and UiO-66(Zr) MOF) can be synthesized by interfacial reaction using a continuous-flow droplet microfluidic system in a single step and one-flow strategy (see, e.g., Jeong et al. (2015) Chem. Mater. 27( 23): 7903-7909.

It is also noted that lipid membrane coated MOFs are described in Cheng et al. (2018) J. Am. Chem. Soc. 140(23): 7282-7291 and Illes et al. (2017) Chem. Mater. 29(19): 8042-8046.

In various embodiments the nanoparticles comprising the drug delivery vehicle described herein can include particles as large (e.g., average or median diameter (or other characteristic dimension) as about 1000 nm. However, in various embodiments the nanoparticles are typically less than 500 nm or less than about 300 nm as, in general, particles larger than 300 nm may be less effective in entering living cells or blood vessel fenestrations. In certain embodiments the nanoparticles range in size from about 30 nm, or from about 40 nm, or from about 50 nm, or from about 60 nm up to about 100 nm, or up to about 90 nm, or up to about 80 nm, or up to about 70 nm. In certain embodiments the nanoparticles range in size from about 60 nm to about 70 nm. Some embodiments include nanoparticles having an average maximum dimension between about 50 nm and about 1000 nm. Other embodiments include nanoparticles having an average maximum dimension between about 50 nm and about 500 nm. Other embodiments include nanoparticles having an average maximum dimension between about 50 nm and about 200 nm. In some embodiments, the average maximum dimension is greater than about 20 nm, greater than about 30 nm, greater than 40 nm, or greater than about 50 nm. Other embodiments include nanoparticles having an average maximum dimension less than about 500 nm, less than about 300 nm, less than about 200 nm, less than about 100 nm or less than about 75 nm. As used herein, the size of the nanoparticle refers to the average or median size of the primary particles, as measured by transmission electron microscopy (TEM) or similar visualization technique.

In certain embodiments the drug delivery vehicles have an average hydrodynamic diameter ranging from about 20 nm or from about 25 nm, or from about 30 nm, or from about 40 nm 50 nm up to about 300 nm, or from about 30 nm up to about 200 nm, or from about 30 up to about 170 nm, or from about 30 nm up to about 150 nm, or from about 30 up to about 100 nm, or from about 50 up to about 80 nm, or from about 30 up to about 70 nm, or from about 60 up to about 70 nm by DLS. In certain embodiments the drug delivery vehicles have an average hydrodynamic diameter ranging from about 145 nm up to about 165 nm by DLS or from about 150 nm up to about 161 nm by DLS. In certain embodiments the nanoparticle comprising the drug delivery vehicle have an average pore size that ranges from about 1 to about 20 nm, or from about 1 to about 10 nm, or from about 1 to about 5 nm, or from about 1 to about 4 nm, or from about 1 to about 3 nm, or from about 2 to about 3 nm.

Illustrative mesoporous silica nanoparticles include, but are not limited to MCM-41, MCM-48, and SBA-15 (see, e.g., Katiyaret al. (2006) J. Chromatog. 1122(1-2): 13-20).

Using the teachings provided herein, nanoparticles comprising the metal organic framework nanoparticles described above, and/or other MOFs are readily available to those of skill in the art and, using the teaching described herein, can be used in the fabrication of the drug delivery vehicles described herein.

### Lipid Bilayer (LB)

The drug carrier nanoparticles described herein comprise a nanoparticle comprising one or more cavities, e.g., a porous nanoparticle such as a mesoporous silica nanoparticle (MSNP)), coated with a lipid bilayer. In certain embodiments the bilayer composition is optimized to provide a rapid and uniform particle coating, to provide colloidal and circulatory stability, and to provide effective cargo retention, while also permitting a desirable cargo release profile.

In certain embodiments the lipid bilayer comprises a combination of a phospholipid, and cholesterol, and in certain embodiments, a pegylated lipid (e.g., PE-PEG₂₀₀₀, DSPE-PEG₂₀₀₀), or a factionalized pegylated lipid (e.g., DSPE-PEG₂₀₀₀-maleimide) to facilitate conjugation with targeting or other moieties.

In certain embodiments, to attach a surface LB coating, a coated lipid film procedure can be utilized in which nanoparticle (e.g., MSNP) suspensions are added to a large lipid film surface, coated on, e.g., a round-bottom flask. Using different lipid bilayer compositions, a series of experiments can be performed to find a composition and optimal lipid/particle ratio that provides rapid and uniform particle wrapping, coating and effective cargo retention and/or release upon sonication. It is believed that this lipid composition and wrapping cannot be achieved by liposomal fusion to the particle surface under low energy vortexing conditions.

In certain illustrative, but non-limiting embodiments the lipid bilayer can comprise: 1) one or more saturated fatty acids with C14-C20 carbon chain, such as phosphatidylethanolamine (PE), 1, 2-distearoyl-sn-glycero-3-phosphoethanolamine (DSPE), dimyristoylphosphatidylcholine (DMPC), dipalmitoylphosphatidylcholine (DPPC), distearoylphosphatidylcholine (DSPC), and diactylphosphatidylcholine (DAPC); and/or 2) One or more unsaturated fatty acids with a C14-C20 carbon chain, such as 1,2-dimyristoleoyl-sn-glycero-3-phosphocholine, 1,2-dipalmitoleoyl-sn-glycero-3-phosphocholine, 1,2-dioleoyl-sn-glycero-3-phosphocholine (DOPC), 1,2-dieicosenoyl-sn-glycero-3-phosphocholine; and/or 3) natural lipids comprising a mixture of fatty acids with C12-C20 carbon chain, such as Egg PC, and Soy PC, sphingomyelin, and 4) cholesterol (CHOL) and/or a modified cholesterol (e.g., cholesterol hemisuccinate (CHEMS)) the like. It is noted that, in certain embodiments, in order to compensate a positive charge, it is possible to use cholesteryl hemisuccinate (CHEMS) that carries one negative charge at pH >6.5 in the formulation. These lipids are illustrative but non-limiting and numerous other lipids are known and can be incorporated into a lipid bilayer for formation of a drug delivery nanocarrier (e.g., a bilayer-coated nanoparticle).

In certain embodiments the drug carrier comprises bilayer comprising a lipid (e.g., a phospholipid), cholesterol, and a PEG functionalized lipid (e.g., a mPEG phospholipid). In certain embodiments the mPEG phospholipids comprises a C14-C18 phospholipid carbon chain from, and a PEG molecular weight from 350-5000 (e.g., MPEG 5000, MPEG 3000, MPEG 2000, MPEG 1000, MPEG 750, MPEG 550, MPEG 350, and the like). In certain embodiments the mPEG phospholipid comprises DSPE-PEG5000, DSPE-PEG3000, DSPE-PEG2000, DSPE-PEG1000, DSPE-PEG750, DSPE-PEG550, DSPE-PEG350, PE-PEG5000, PE-PEG3000, PE-PEG2000, PE-PEG1000, PE-PEG750, PE-PEG550, PE-PEG350, and the like. MPEGs are commercially available (see, e.g., //avantilipids.com/product-category/products/polymers-polymerizable-lipids/mpeg-phospholipids).

In certain embodiments lipid bilayer comprises an mPEG phospholipid with a phospholipid C14-C18 carbon chain, and a PEG molecular weight ranging from about 350 Da to 5000 Da. In certain embodiments the lipid bilayer comprises PE-PEG_{2K}.

In certain embodiments the lipid bilayer comprises DPSC, cholesterol, and DSPE-PEG. In certain embodiments, the molar ratio of DPSC : cholesterol : PE-PEG ranges from 20-90% DSPC : 10%-50% Chol : 1%-10% DS PE-PEG. In certain embodiments, the molar ratio of DSPC : Chol :DS PE-PEG is about 3 : 2 : 0.15. In certain embodiments, the nanoparticles have a particle (e.g., MSNP:lipid ratio of 1:1.25 (w/w). In certain embodiments, the lipid bilayer comprises a cholesterol derivative selected from the group consisting of cholesterol hemisuccinate (CHEMS), lysine-based cholesterol (CHLYS), and PEGylated cholesterol (Chol-PEG). In certain embodiments, the cholesterol derivative is in place of said cholesterol. In certain embodiments, the lipid bilayer comprises CHEMS. In certain embodiments, the lipid bilayer comprises CHEMS ranging from about 5% (mol percent) up to about 30% total lipid. In certain embodiments, the lipid bilayer comprises about 10% or about 20% CHEMS or about 30% CHEMS or about 40% CHEMS.

The foregoing lipid bilayer formulations are illustrative and non-limiting. Using the teaching provided herein numerous other lipid bilayer formulations suitable for use in the methods described herein will be available to one of skill in the art.

### Remote Loading of Drug Delivery Vehicles

In certain embodiments, the encapsulation of the camptothecin analog (e.g., irinotecan) inhibitor (and other agents when present, e.g., autophagy inhibitor(s)) in the nanoparticle can accomplished by using a “remote loading” strategy in which the addition of the drug (e.g., camptothecin analog such as irinotecan) to LB-coated nanoparticles which achieves high loading levels using a pH gradient or an ion gradient capable of generating a pH gradient (see, e.g., Ogawa et al. (2009) J. Control. Rel. 1(5): 4-10; Fritze et al. (2006) Biochimica et Biophys Acta. 1758: 1633-1640). In general, the remote loading method involves adding a cargo-trapping reagent (e.g., a protonating reagent such as ammonium sulfate, TEA₈SOS, etc.) which can be added to the lipid biofilm prior to the sonication in the formation of LB coated nanoparticles.

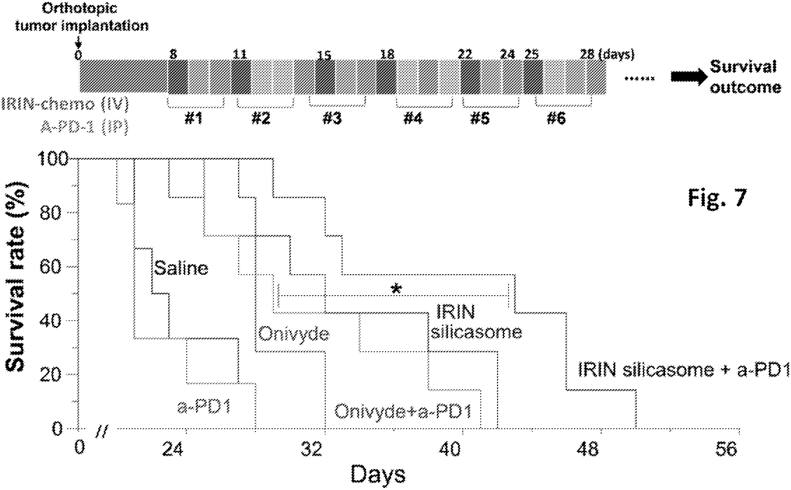

Thus for example, in one illustrative, but non-limiting embodiment, porous nanoparticles (e.g., mesoporous silica nanoparticles) containing the trapping agent (e.g., TEA₈SOS) are incubated in an IRIN solution, allowing the amphipathic drug to diffuse across the lipid bilayer. Proton release from the trapping agent converts the encapsulated IRIN to a hydrophilic derivative that cannot back-diffuse across the LB (see, e.g., FIG. 7).

In one illustrative, but non-limiting embodiment, 40 mg/mL of the purified MSNPs, exposed to 80 mM TEA₈SOS solution for soaking in, are added to an ~50% (w/v) lipid solution. In this illustrative embodiment, this ethanol suspended solution contains DSPC/Chol/DSPE-PEG₂₀₀₀, in the molar ratio of 3:2:0.15. This yields a MSNP:lipid ratio of 1:1.25 (w/w). The suspension can be introduced by a flow pump into a flow cell that provides probe sonication (e.g., Ultrasonic Processor Model VCX500, 80% amplitude) at a 15 s/15s on/off cycle and a flow rate of 5 mL/min⁴⁸. In order to remove the free trapping agent, the sample can readily be purified through centrifugation (4,000 rpm for 5 min), followed by purification, using size exclusion chromatography. For IRIN import, TEA₈SOS-loaded particles are mixed with IRIN and incubated at, e.g., 65° C. for about 1 hour. The IRIN silicasomes can be purified and filtered across a 0.2 µm filter for sterilization.

Of course, this protocol is illustrative and non-limiting. Using this teaching, numerous other nanoparticle drug carriers comprising a nanoparticle surrounded by a lipid bilayer can be produced by one of skill in the art.

As explained above, in certain embodiments a cargo-trapping reagent (e.g., protonating agent) can be utilized to facilitate incorporation of the camptothecin analog (e.g., IRIN) and in certain embodiments an additional cargo (e.g., an autophagy inhibitor) into the LB coated nanoparticle.

In certain embodiments the cargo-trapping reagent can be selected to interact with a desired cargo. In some embodiments, this interaction can be an ionic or protonation reaction, although other modes of interaction are contemplated. The cargo-trapping agent can have one or more ionic sites, i.e., can be mono-ionic or poly-ionic. The ionic moiety can be cationic, anionic, or in some cases, the cargo-trapping agent can include both cationic and anionic moieties. The ionic sites can be in equilibrium with corresponding uncharged forms; for example, an anionic carboxylate (—COO⁻) can be in equilibrium with its corresponding carboxylic acid (—COOH); or in another example, an amine (—NH₂) can be in equilibrium with its corresponding protonated ammonium form (—NH₃⁺). These equilibriums are influenced by the pH of the local environment.

Likewise, in certain embodiments, the cargo, e.g., a camptothecin analog can include one or more ionic sites. The cargo-trapping agent and cargo can be selected to interact inside the LB coated nanoparticle. This interaction can help retain the cargo within the nanoparticle until release of the cargo is desired. In some embodiments, the cargo can exist in a pH-dependent equilibrium between non-ionic and ionic forms. The non-ionic form can diffuse across the lipid bilayer and enter the vesicle or the pores of the MSNP. There, the cargo-trapping agent (e.g., a polyionic cargo-trapping agent) can interact with the ionic form of the cargo and thereby retain the cargo within the nanoparticle comprising the drug delivery vehicle, e.g., within the vesicle or within the pores of the nanoparticle (provided the ionic forms of the cargo and cargo-trapping agent have opposite charges). The interaction can be an ionic interaction and can include formation of a precipitate. Trapping of cargo within the nanoparticle can provide higher levels of cargo loading compared to similar systems, e.g., drug delivery vehicles that omit the cargo-trapping agent, or liposomes that do include a trapping agent. Release of the cargo can be achieved by an appropriate change in pH to disrupt the interaction between the cargo and cargo-trapping agent, for example, by returning the cargo to its non-ionic state which can more readily diffuse across the lipid bilayer

The cargo trapping agent need not be limited to ammonium sulfate. In certain embodiments the cargo trapping comprises molecules like TEA₈SOS, citric acid, (NH₄)₂SO₄, and the like. Other trapping agents include, but are not limited to, ammonium salts (e.g., ammonium sulfate, ammonium sucrose octasulfate, ammonium α-cyclodextrin sulfate, ammonium β-cyclodextrin sulfate, ammonium γ-cyclodextrin sulfate, ammonium phosphate, ammonium α-cyclodextrin phosphate, ammonium β-cyclodextrin phosphate, ammonium γ-cyclodextrin phosphate, ammonium citrate, ammonium acetate, and the like), trimethylammonium salts (e.g., trimethylammonium sulfate, trimethylammonium sucrose octasulfate, trimethylammonium α-cyclodextrin sulfate, trimethylammonium β-cyclodextrin sulfate, trimethylammonium γ-cyclodextrin sulfate, trimethylammonium phosphate, trimethylammonium α-cyclodextrin phosphate, trimethylammonium β-cyclodextrin phosphate, trimethylammonium γ-cyclodextrin phosphate, trimethylammonium citrate, trimethylammonium acetate, and the like), triethylammonium salts (e.g., triethylammonium sulfate, triethylammonium sucrose octasulfate, triethylammonium α-cyclodextrin sulfate, triethylammonium β-cyclodextrin sulfate, triethylammonium γ-cyclodextrin sulfate, triethylammonium phosphate, triethylammonium α-cyclodextrin phosphate, triethylammonium β-cyclodextrin phosphate, triethylammonium γ-cyclodextrin phosphate, triethylammonium citrate, triethylammonium acetate, and the like).

It is also worth pointing out that transmembrane pH gradients can also be generated by acidic buffers (e.g. citrate) (Chou et al. (2003) J. Biosci. Bioengineer., 95(4): 405-408; Nichols et al. (1976) Biochimica et Biophysica Acta (BBA)-Biomembranes, 455(1): 269-271), proton-generating dissociable salts (e.g. (NH₄)₂SO₄) (Haran et al. (1993) Biochimica et Biophysica Acta (BBA)-Biomembranes, 1151(2): 201-215; Maurer-Spurej et al. (1999) Biochimica et Biophysica Acta (BBA)-Biomembranes, 1416(1): 1-10; Fritze et al. (2006) Biochimica et Biophysica Acta (BBA)-Biomembranes, 1758(10): 1633-1640), or ionophore-mediated ion gradients from metal salts (e.g. A23187 and MnSO₄) (Messerer et al. (2004) Clinical Cancer Res. 10(19): 6638-6649; Ramsay et al. (2008) Eur. J. Pharmaceut. Biopharmaceut. 68(3): 607-617; Fenske et al. (1998) Biochimica et Biophysica Acta (BBA)-Biomembranes, 1414(1): 188-204). Moreover, it is possible to generate reverse pH gradients for drug loading, such as use a calcium acetate gradient to improve amphiphilic weak acid loading in LB-MSNP, a strategy that has been utilized in liposomes (Avnir et al. (2008) Arthritis & Rheumatism, 58(1): 119-129).

Using the teachings provided herein, numerous other agents (e.g., other camptothecin analogs and/or various autophagy inhibitors) can be remote loaded (e.g., loaded using a cargo trapping agent) into the nanoparticle drug delivery systems described herein.

### Targeting Ligands

In certain embodiments the drug delivery vehicles described herein can be conjugated to one or more targeting ligands, e.g., to facilitate specific delivery in endothelial cells, to cancer cells, to fusogenic ligands, e.g., to facilitate endosomal escape, ligands to promote transport across the blood-brain barrier, and the like.

In one illustrative, but non-limiting embodiment, the delivery vehicles described herein is conjugated to a fusogenic peptide such as histidine-rich H5WYG (H₂N-GLFHAIAHFIHGGWHGLIHGWYG-COOH, (SEQ ID NO:1)) (see, e.g., Midoux et al., (1998) Bioconjug. Chem. 9: 260-267).

In certain embodiments delivery vehicles described herein are conjugated to one or more targeting ligand(s) that can include antibodies as well as targeting peptides. Targeting antibodies include, but are not limited to intact immunoglobulins, immunoglobulin fragments (e.g., F(ab)′₂, Fab, etc.) single chain antibodies, diabodies, affibodies, unibodies, nanobodies, and the like. In certain embodiments antibodies will be used that specifically bind a cancer marker (e.g., a tumor associated antigen). A wide variety of cancer markers are known to those of skill in the art. The markers need not be unique to cancer cells, but can also be effective where the expression of the marker is elevated in a cancer cell (as compared to normal healthy cells) or where the marker is not present at comparable levels in surrounding tissues (especially where the chimeric moiety is delivered locally).

Illustrative cancer markers include, for example, the tumor marker recognized by the ND4 monoclonal antibody. This marker is found on poorly differentiated colorectal cancer, as well as gastrointestinal neuroendocrine tumors (see, e.g., Tobi et al. (1998) Cancer Detection and Prevention, 22(2): 147-152). Other important targets for cancer immunotherapy are membrane bound complement regulatory glycoproteins CD46, CD55 and CD59, which have been found to be expressed on most tumor cells in vivo and in vitro. Human mucins (e.g. MUC1) are known tumor markers as are gp100, tyrosinase, and MAGE, which are found in melanoma. Wild-type Wilms’ tumor gene WT1 is expressed at high levels not only in most of acute myelocytic, acute lymphocytic, and chronic myelocytic leukemia, but also in various types of solid tumors including lung cancer.

Acute lymphocytic leukemia has been characterized by the TAAs HLA-Dr, CD1, CD2, CD5, CD7, CD19, and CD20. Acute myelogenous leukemia has been characterized by the TAAs HLA-Dr, CD7, CD13, CD14, CD15, CD33, and CD34. Breast cancer has been characterized by the markers EGFR, HER2, MUC1, Tag-72. Various carcinomas have been characterized by the markers MUC1, TAG-72, and CEA. Chronic lymphocytic leukemia has been characterized by the markers CD3, CD19, CD20, CD21, CD25, and HLA-DR. Hairy cell leukemia has been characterized by the markers CD19, CD20, CD21, CD25. Hodgkin’s disease has been characterized by the Leu-M1 marker. Various melanomas have been characterized by the HMB 45 marker. Non-Hodgkins lymphomas have been characterized by the CD20, CD19, and Ia marker. And various prostate cancers have been characterized by the PSMA and SE10 markers.

In addition, many kinds of tumor cells display unusual antigens that are either inappropriate for the cell type and/or its environment, or are only normally present during the organisms’ development (e.g., fetal antigens). Examples of such antigens include the glycosphingolipid GD2, a disialoganglioside that is normally only expressed at a significant level on the outer surface membranes of neuronal cells, where its exposure to the immune system is limited by the blood-brain barrier. GD2 is expressed on the surfaces of a wide range of tumor cells including neuroblastoma, medulloblastomas, astrocytomas, melanomas, small-cell lung cancer, osteosarcomas and other soft tissue sarcomas. GD2 is thus a convenient tumor-specific target for immunotherapies.

Other kinds of tumor cells display cell surface receptors that are rare or absent on the surfaces of healthy cells, and which are responsible for activating cellular signaling pathways that cause the unregulated growth and division of the tumor cell. Examples include (ErbB2) HER2/neu, a constitutively active cell surface receptor that is produced at abnormally high levels on the surface of breast cancer tumor cells.

Other useful targets include, but are not limited to CD20, CD52, CD33, epidermal growth factor receptor and the like.

An illustrative, but not limiting list of suitable tumor markers is provided in Table 3. Antibodies to these and other cancer markers are known to those of skill in the art and can be obtained commercially or readily produced, e.g. using phage-display technology. Such antibodies can readily be conjugated to the drug delivery vehicles (e.g., LB-coated nanoparticle) described herein, e.g., in the same manner that iRGD peptide is conjugated in Example 3.

TABLE 3

Illustrative cancer markers and associated references, all of which are incorporated herein by reference for the purpose of identifying the referenced tumor markers

| | |
|---|---|
| Marker | Reference |
| 5 alpha reductase | Délos et al. (1998) Int J Cancer, 75:6 840-846 |
| α-fetoprotein | Esteban et al. (1996) Tumour Biol., 17(5): 299-305 |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (`998) J Exp Med., 188(6): 1185-1190 |
| BAGE | Böel et al. (1995) Immunity, 2: 167-175. |
| β-catenin | Hugh et al. (1999) Int J Cancer, 82(4): 504-11 |
| Bc12 | Koty et al. (1999) Lung Cancer, 23(2): 115-127 |
| bcr-abl (b3a2) | Verfaillie et al. (‘996) Blood, 87(11): 4770-4779 |
| CA-125 | Bast et al. (`998) Int J Biol Markers, 13(4): 179-187 |
| CASP-8/FLICE | Mandruzzato et al. (1997) J Exp Med., 186(5): 785-793. |
| Cathepsins | Thomssen et al.(1995) Clin Cancer Res., 1(7): 741-746 |
| CD19 | Scheuermann et al. (1995) Leuk Lymphoma, 18(5-6): 385-397 |
| CD20 | Knox et al. (1996) Clin Cancer Res., 2(3): 457-470 |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD33 | Nakase et al. (1996) Am J Clin Pathol., 105(6): 761-768 |
| CD35 | Yamakawa et al. Cancer, 73(11): 2808-2817 |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD59 (791Tgp72) | Jarvis et al. (1997) Int J Cancer, 71(6): 1049-1055 |
| CDC27 | Wang et al. (1999) Science, 284(5418): 1351-1354 |
| CDK4 | Wölfel et al. (1995) Science, 269(5228): 1281-1284 |
| CEA | Kass et al. (1999) Cancer Res., 59(3): 676-683 |
| c-myc | Watson et al. (1991) Cancer Res., 51(15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al. (1992) J Biol Chem., 267(33): 24140-72414 |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing hormone (bombesin) | Wang et al. (1996) Int J Cancer, 68(4): 528-534 |
| GD2/GD3/GM2 | Wiesner and Sweeley (1995) Int J Cancer, 60(3): 294-299 |
| GnRH | Bahk et al.(1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunother., 44(4): 239-247 |
| gp-100-in4 | Kirkin et al. (1998) APMIS, 106(7): 665-679 |
| gp15 | Maeurer et al.(1996) Melanoma Res., 6(1): 11-24 |
| gp75/TRP-1 | Lewis et al.(1995) Semin Cancer Biol., 6(6): 321-327 |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| HMTV | Kahl et al.(1991) Br J Cancer, 63(4): 534-540 |
| Hsp70 | Jaattela et al. (1998) EMBO J., 17(21): 6124-6134 |
| hTERT (telomerase) | Vonderheide et al. (1999) Immunity, 10: 673-679. 1999. |
| IGFR1 | Ellis et al. (1998) Breast Cancer Res. Treat., 52: 175-184 |
| IL-13R | Murata et al. (1997) Biochem Biophys Res Commun., 238(1): 90-94 |
| iNOS | Klotz et al. (1998) Cancer, 82(10): 1897-1903 |
| Ki 67 | Gerdes et al. (1983) Int J Cancer, 31: 13-20 |
| KIAA0205 | Guéguen et al. (1998) J Immunol., 160(12): 6188-6194 |
| K-ras, H-ras, N-ras | Abrams et al. (1996) Semin Oncol., 23(1): 118-134 |
| KSA (CO17-1A) | Zhang et al. (1998) Clin Cancer Res., 4(2): 295-302 |
| LDLR-FUT | Caruso et al. (1998) Oncol Rep., 5(4): 927-930 |
| MAGE Family (MAGE1, MAGE3, etc.) | Marchand et al. (1999) Int J Cancer, 80(2): 219-230 |
| Mammaglobin | Watson et al. (1999) Cancer Res., 59: 13 3028-3031 |
| MAP17 | Kocher et al. (1996) Am J Pathol., 149(2): 493-500 |
| Melan-A/MART-1 | Lewis and Houghton (1995) Semin Cancer Biol., 6(6): 321-327 |
| mesothelin | Chang et al. (1996) Proc. Natl. Acad. Sci., USA, 93(1): 136-140 |
| MIC A/B | Groh et al.(1998) Science, 279: 1737-1740 |
| MT-MMP’s, such as MMP2, MMP3, MMP7, MMP9 | Sato and Seiki (1996) J Biochem (Tokyo), 119(2): 209-215 |
| Mox1 | Candia et al. (1992) Development, 116(4): 1123-1136 |
| Mucin, such as MUC-1, MUC-2, MUC-3, and MUC-4 | Lewis and Houghton (1995) Semin Cancer Biol., 6(6): 321-327 |
| MUM-1 | Kirkin et al. (1998) APMIS, 106(7): 665-679 |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grøndahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PRAME | Kirkin et al. (1998) APMIS, 106(7): 665-679 |
| Probasin | Matuo et al. (1985) Biochem Biophys Res Commun., 130(1): 293-300 |
| Progenipoietin | ---- |
| PSA | Sanda et al. (1999) Urology, 53(2): 260-266. |
| PSM | Kawakami et al.(1997) Cancer Res., 57(12): 2321-2324 |
| RAGE-1 | Gaugler et al.(1996) Immunogenetics, 44(5): 323-330 |
| Rb | Dosaka-Akita et al. (1997) Cancer, 79(7): 1329-1337 |
| RCAS1 | Sonoda et al.(1996) Cancer, 77(8): 1501-1509. |
| SART-1 | Kikuchi et al.(1999) Int J Cancer, 81(3): 459-466 |
| SSX gene Family | Gure et al. (1997) Int J Cancer, 72(6): 965-971 |
| STAT3 | Bromberg et al. (1999) Cell, 98(3): 295-303 |
| STn (mucin assoc.) | Sandmaier et al. (1999) J Immunother., 22(1): 54-66 |
| TAG-72 | Kuroki et al. (1990)Cancer Res., 50(16): 4872-4879 |
| TGF-α | Imanishi et al. (1989) Br J Cancer, 59(5): 761-765 |
| TGF-β | Picon et al. (1998) Cancer Epidemiol Biomarkers Prey, 7(6): 497-504 |
| Thymosin β 15 | Bao et al. (1996) Nature Medicine. 2(12), 1322-1328 |
| IFN-α | Moradi et al. (1993) Cancer, 72(8): 2433-2440 |
| TPA | Maulard et al. (1994) Cancer, 73(2): 394-398 |
| TPI | Nishida et al. (1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| ZAG | Sanchez et al. (1999) Science, 283(5409): 1914-1919 |
| p16INK4 | Quelle et al. (1995) Oncogene Aug. 17, 1995; 11(4): 635-645 |
| Glutathione S-transferase | Hengstler (1998) et al. Recent Results Cancer Res., 154: 47-85 |

Any of the foregoing markers can be used as targets for the targeting moieties comprising delivery vehicles described herein. In certain embodiments the target markers include, but are not limited to members of the epidermal growth factor family (e.g., HER2, HER3, EGF, HER4), CD1, CD2, CD3, CD5, CD7, CD13, CD14, CD15, CD19, CD20, CD21, CD23, CD25, CD33, CD34, CD38, 5E10, CEA, HLA-DR, HM 1.24, HMB 45, 1a, Leu-M1, MUC1, PMSA, TAG-72, phosphatidyl serine antigen, and the like.

The foregoing markers are intended to be illustrative and not limiting. Other tumor associated antigens will be known to those of skill in the art.

Where the tumor marker is a cell surface receptor, a ligand to that receptor can function as targeting moieties. Similarly, mimetics of such ligands can also be used as targeting moieties. Thus, in certain embodiments peptide ligands, and other ligands, can be used in addition to or in place of various antibodies. An illustrative, but non-limiting list of suitable targeting ligands is shown in Table 4. In certain embodiments any one or more of these peptides can be conjugated to a drug delivery vehicle described herein.

TABLE 4

Illustrative, but non-limiting ligands that target membrane receptors expressed or overexpressed by various cancer cells

| Target Membrane Receptor | Targeting ligand | SEQ ID NO |
|---|---|---|
| Integrin receptor Aᵥβ₃ | c(RGDfK) | 2 |
| | c(RGDfC) | 3 |
| | c(RGDyC) | 4 |
| | RGD | |
| GFR | GE11 (YHWYGYTPQNVI) | 5 |
| GFR | GSG-KCCYSL | 6 |
| SSTR2 | Ostreotide | |
| GRP | QWAVGHML | 7 |
| CCK | DYMGWMDF | 8 |
| NT | RRPYIL | 9 |
| | RRPYILQLYENKPRRPYIL | 10 |
| LHRH | Gondaorelin | |
| GPRC family members | Antagonist G | |
| Tumor Cell Receptor | Targeting Ligand | |
| TfR | Tf | |
| EGFR | EGF | |
| FAR (FR-α) | FA | |
| FR-α | Methotrexate | |
| Sigma receptor | Anisamide | |
| Importing α and β receptors | TAT peptides | |
| IL-13Rα2 | IL-13 peptide | |
| HER2 | Anti-herceptin | |
| HER2/neu | Anti-HER2/neu | |
| ErbB2 | Anti-ErbB2 | |
| Mesothelin | Anti-ME1 | |
| CD105/endoglin | Anti-TRC105 | |
| NET | MABG | |
| NRP-1 | RGD-type peptide (RDGRC) | 11 |
| SA | ConA | |
| CD44 | HA | |
| α_{ν}β₃ integrins | c(RGDyK) | 12 |
| αᵥβ₃ integrins | cRGD | |
| α_{ν}β₃ integrins | KKKKKKKRGD c-RGDFK | 13 14 |
| α_{ν}β₃ integrins | KKKKKKKKRGDRGD | 15 |
| α_{ν}β₃ integrins | NNNGPLGRGRGDK-Ad | 16 |
| α_{ν}β₃ integrins | NNNRGDFFFFC | 17 |
| α_{ν}β₃ integrins | Thiolated-RGD | |
| (VCAM-1)R | Anti-(VCAM-1) | |
| VEGFR | VEGF | |

Tf: Transferrin;
FA: Folic acid;
EGFR: Epidermal growth factor;
TAT: Transactivator of transcription;
IL-13: Interleukin-13;
MABG: metaaminobenzyl guanidine (meta-iodobenzylguanidine analogue);
ConA: concanavalin A;
c(RGD): Cyclic RGD;
VCAM-1: vascular cell adhesion molecule 1;
VEGFR: Vascular endothelial growth factor;
TfR: transferrin receptor;
EGFR: epidermal growth factor receptor;
FAR (FR-α): Folic acid receptor;
IL-13Rα2: interleukin-13 receptor subunit alpha-2;
HER2: epidermal growth factor receptor;
ErbB2: Receptor tyrosine-protein kinase 2;
NET: norepinephrine transporter;
NRP-1: neuropilin receptors;
SA: sialic acid;
(VCAM-1)R: vascular cell adhesion molecule 1 receptor;
VE b: Vascular endothelial growth factor receptor;
GRP: Gastrin releasing peptide receptor;
NT: neurotensin receptor;
CCK: Cholecystokinin receptor;
c() indicates cyclopeptide.
Lower case indicates “D” amino acid.

In certain embodiments the nanoparticle drug delivery vehicles described herein can be conjugated to moieties that facilitate stability in circulation and/or that hide the drug delivery vehicle from the reticuloendothelial system (REC) and/or that facilitate transport across a barrier (e.g., a stromal barrier, the blood brain barrier, etc.), and/or into a tissue. In certain embodiments the drug delivery vehicle are conjugated to transferrin or ApoE to facilitate transport across the blood brain barrier. In certain embodiments the drug delivery vehicle are conjugated to folate.

Methods of coupling the nanoparticle drug delivery vehicle to targeting (or other) agents are well known to those of skill in the art. Examples include, but are not limited to the use of biotin and avidin or streptavidin (see, e.g., U.S. Pat. No: US 4,885,172 A), by traditional chemical reactions using, for example, bifunctional coupling agents such as glutaraldehyde, diimide esters, aromatic and aliphatic diisocyanates, bis-p-nitrophenyl esters of dicarboxylic acids, aromatic disulfonyl chlorides and bifunctional arylhalides such as 1,5-difluoro-2,4-dinitrobenzene; p,p′-difluoro m,m′-dinitrodiphenyl sulfone, sulfhydryl-reactive maleimides, and the like. Appropriate reactions which may be applied to such couplings are described in Williams et al. Methods in Immunology and Immunochemistry Vol. 1, Academic Press, New York 1967.

In one illustrative but non-limiting approach a peptide (e.g., iRGD) is coupled to the nanoparticle drug delivery vehicle by a lipid coupled to a linker (e.g., DSPE-PEG₂₀₀₀maleimide), allowing thiol-maleimide coupling to the cysteine-modified peptide. It will also be recognized that in certain embodiments the targeting (and other) moieties can be conjugated to other moieties comprising the lipid bilayer. In certain embodiments possible to improve tumor delivery of the nanoparticle drug delivery vehicle through co-administration (not conjugated) of the iRGD peptide to enhance particle transcytosis.

The former conjugates and coupling methods are illustrative and non-limiting. Using the teachings provided herein, numerous other moieties can be conjugated to the nanoparticle drug delivery vehicles described herein by any of a variety of methods.

### Pharmaceutical Formulations, Administration and Therapy

In some embodiments, the nanoparticle drug delivery vehicles described herein are administered alone or in a mixture with a physiologically-acceptable carrier (such as physiological saline or phosphate buffer) selected in accordance with the route of administration and standard pharmaceutical practice. For example, when used as an injectable, the nanoparticle drug delivery vehicles can be formulated as a sterile suspension, dispersion, or emulsion with a pharmaceutically acceptable carrier. In certain embodiments normal saline can be employed as the pharmaceutically acceptable carrier. Other suitable carriers include, e.g., water, buffered water, 0.4% saline, 0.3% glycine, 5% glucose and the like, including glycoproteins for enhanced stability, such as albumin, lipoprotein, globulin, etc. In compositions comprising saline or other salt-containing carriers, the carrier is preferably added following nanoparticle drug delivery vehicle formation. Thus, after the nanoparticle drug delivery vehicle is formed and loaded with suitable drug(s), the vehicles can be diluted into pharmaceutically acceptable carriers such as normal saline.

In certain embodiments, the nanoparticle drug delivery vehicles described herein (containing one or more camptothecin analogs) are provided in a compound drug formulation that additionally comprises one or more checkpoint inhibitors. Methods of making combined formulations are well known to those of skill in the art. For example, in certain embodiments a capsule can provide two chambers where the checkpoint inhibitor(s) and the nanoparticle drug delivery vehicle containing the camptothecin analog(s) are each in their own chamber. Alternatively, a table can be provided where the checkpoint inhibitor(s) and the nanoparticle drug delivery vehicle containing the camptothecin analog(s) are located in different layers in the tablet. As an injectable, in certain embodiments, the checkpoint inhibitor(s) and the nanoparticle drug delivery vehicle containing the camptothecin analog(s) are provided together in one solution or suspension. These approaches are illustrative and non-limiting. Using the teachings provided herein, numerous other combined formulations will be available to one of skill in the art.

The pharmaceutical compositions may be sterilized by conventional, well-known sterilization techniques. The resulting aqueous solutions, suspensions, dispersions, emulsions, etc., may be packaged for use or filtered under aseptic conditions. In certain embodiments the nanoparticle drug delivery vehicles described herein are lyophilized, the lyophilized preparation being combined with a sterile aqueous solution prior to administration. The compositions may also contain pharmaceutically acceptable auxiliary substances as required to approximate physiological conditions, such as pH-adjusting and buffering agents, tonicity adjusting agents and the like, for example, sodium acetate, sodium lactate, sodium chloride, potassium chloride, calcium chloride, etc.

Additionally, in certain embodiments, the pharmaceutical formulation may include lipid-protective agents that protect lipids against free-radical and lipid-peroxidative damage on storage. Lipophilic free-radical quenchers, such as alpha-tocopherol and watersoluble iron-specific chelators, such as ferrioxamine, are suitable.

The concentration of the nanoparticle drug delivery vehicles and/or checkpoint inhibitor(s) in the pharmaceutical formulations can vary widely, e.g., from less than approximately 0.05%, usually at least approximately 2 to 5% to as much as 10 to 50%, or to 40%, or to 30% by weight and are selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, nanoparticle drug delivery vehicles composed of irritating lipids may be diluted to low concentrations to lessen inflammation at the site of administration. The amount of nanoparticle drug delivery vehicles administered will depend upon the particular drug used, the disease state being treated and the judgment of the clinician but will generally be between approximately 0.01 and approximately 50 mg per kilogram of body weight, preferably between approximately 0.1 and approximately 5 mg per kg of body weight.

In some embodiments, e.g., it is desirable to include polyethylene glycol (PEG)-modified phospholipids in the LB-coated nanoparticles or vesicles. Alternatively, or additionally, in certain embodiments, PEG-ceramide, or ganglioside G_{MI}-modified lipids can be incorporated in the nanoparticle drug delivery vehicles described herein. Addition of such components helps prevent delivery vehicle aggregation and provides for increasing circulation lifetime and increasing the delivery of the loaded delivery vehicles to the target tissues. In certain embodiments the concentration of the PEG-modified phospholipids, PEG-ceramide, or G_{MI}-modified lipids in the nanoparticle drug delivery vehicles will be approximately 1 to 15%.

In some embodiments, overall nanoparticle drug delivery vehicle charge is an important determinant in clearance of the vehicle from the blood. Generally, in various sembodiments, particles with a slight negative charge (e.g.,i-5 mV to -30 mV) are used to provide a longer circulatory half-life, compared to cationic particles that are frequently sequestered away from remaining in the systemic circulation. Drug delivery vehicles with prolonged circulation half-lives are typically desirable for therapeutic uses. For instance, in certain embodiments, drug delivery nanoparticle drug delivery vehicles that are maintained from 8 hrs, or 12 hrs, or 24 hrs, or greater are desirable.

In another example of their use, the nanoparticle drug delivery vehicles can be incorporated into a broad range of topical dosage forms including but not limited to gels, oils, emulsions, and the like, e.g., for the treatment of a topical cancer. For instance, in some embodiments the suspension containing the drug delivery vehicles is formulated and administered as a topical cream, paste, ointment, gel, lotion, and the like.

In some embodiments, pharmaceutical formulations comprising the nanoparticle drug delivery vehicles described herein and/or one or more checkpoint inhibitors additionally incorporate a buffering agent. The buffering agent may be any pharmaceutically acceptable buffering agent. Buffer systems include, but are not limited to citrate buffers, acetate buffers, borate buffers, and phosphate buffers. Examples of buffers include, but are not limited to citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate, benzoic acid, and the like.

In some embodiments, pharmaceutical formulations comprising the nanoparticle drug delivery vehicles described herein and/or one or more checkpoint inhibitors additionally incorporate a chelating agent. The chelating agent may be any pharmaceutically acceptable chelating agent. Chelating agents include, but are not limited to ethylene diaminetetraacetic acid (also synonymous with EDTA, edetic acid, versene acid, and sequestrene), and EDTA derivatives, such as dipotassium edetate, disodium edetate, edetate calcium disodium, sodium edetate, trisodium edetate, and potassium edetate. Other chelating agents include citric acid (e.g., citric acid monohydrate) and derivatives thereof. Derivatives of citric acid include anhydrous citric acid, trisodiumcitrate-dihydrate, and the like. Still other chelating agents include, but are not limited to, niacinamide and derivatives thereof and sodium deoxycholate and derivatives thereof.

In some embodiments, pharmaceutical formulations comprising the nanoparticle drug delivery vehicles described herein and/or one or more checkpoint inhibitors additionally incorporate an antioxidant. The antioxidant may be any pharmaceutically acceptable antioxidant. Antioxidants are well known to those of ordinary skill in the art and include, but are not limited to, materials such as ascorbic acid, ascorbic acid derivatives (e.g., ascorbylpalmitate, ascorbylstearate, sodium ascorbate, calcium ascorbate, etc.), butylated hydroxy anisole, buylated hydroxy toluene, alkylgallate, sodium meta-bisulfate, sodium bisulfate, sodium dithionite, sodium thioglycollic acid, sodium formaldehyde sulfoxylate, tocopherol and derivatives thereof, (d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, sodium sulfite and N-acetyl cysteine. In certain embodiments such materials, when present, are typically added in ranges from 0.01 to 2.0%.

In some embodiments, pharmaceutical formulations comprising the nanoparticle drug delivery vehicles described herein and/or one or more checkpoint inhibitors are formulated with a cryoprotectant. The cryoprotecting agent may be any pharmaceutically acceptable cryoprotecting agent. Common cryoprotecting agents include, but are not limited to, histidine, polyethylene glycol, polyvinyl pyrrolidine, lactose, sucrose, mannitol, polyols, and the like.

In some embodiments, pharmaceutical formulations comprising the nanoparticle drug delivery vehicles described herein and/or one or more checkpoint inhibitors are formulated with an isotonic agent. The isotonic agent can be any pharmaceutically acceptable isotonic agent. This term is used in the art interchangeably with iso-osmotic agent, and is known as a compound that is added to the pharmaceutical preparation to increase the osmotic pressure, e.g., in some embodiments to that of 0.9% sodium chloride solution, which is iso-osmotic with human extracellular fluids, such as plasma. Illustrative isotonicity agents include, but are not limited to, sodium chloride, mannitol, sorbitol, lactose, dextrose and glycerol.

In certain embodiments pharmaceutical formulations of the nanoparticle drug delivery vehicles described herein and/or one or more checkpoint inhibitors may optionally comprise a preservative. Common preservatives include, but are not limited to, those selected from the group consisting of chlorobutanol, parabens, thimerosol, benzyl alcohol, and phenol. Suitable preservatives include but are not limited to: chlorobutanol (e.g., 0.3-0.9% w/v), parabens (e.g., 0.01-5.0%), thimerosal (e.g., 0.004-0.2%), benzyl alcohol (e.g., 0.5-5%), phenol (e.g., 0.1-1.0%), and the like.

In some embodiments, pharmaceutical formulations comprising the nanoparticle drug delivery vehicles described herein and/or one or more checkpoint inhibitors are formulated with a humectant, e.g., to provide a pleasant mouth-feel in oral applications. Humectants known in the art include, but are not limited to, cholesterol, fatty acids, glycerin, lauric acid, magnesium stearate, pentaerythritol, and propylene glycol.

In some embodiments, an emulsifying agent is included in the formulations, for example, to ensure complete dissolution of all excipients, especially hydrophobic components such as benzyl alcohol. Many emulsifiers are known in the art, e.g., polysorbate 60.

For some embodiments related to oral administration, it may be desirable to add a pharmaceutically acceptable flavoring agent and/or sweetener. Compounds such as saccharin, glycerin, simple syrup, and sorbitol are useful as sweeteners.

### Administration

The nanoparticle drug delivery vehicles described herein and/or one or more checkpoint inhibitors can be administered to a subject (e.g., patient) by any of a variety of techniques.

In certain embodiments the nanoparticle drug delivery vehicles and/or pharmaceutical formulations thereof and/or one or more checkpoint inhibitors are administered parenterally, e.g., intraarticularly, intravenously, intraperitoneally, subcutaneously, or intramuscularly. In some embodiments, the pharmaceutical compositions are administered intravenously, intraarterially, or intraperitoneally by a bolus injection (see, e.g., U.S. Pat. Nos. 3,993,754; 4,145,410; 4,235,871; 4,224,179; 4,522,803; and 4,588,578 describing administration of liposomes). Particular pharmaceutical formulations suitable for this administration are found in Remington’s Pharmaceutical Sciences, Mack Publishing Company, Philadelphia, Pa., 17th ed. (1985). Typically, the formulations comprise a solution of the nanoparticle drug delivery vehicles suspended in an acceptable carrier, preferably an aqueous carrier. As noted above, suitable aqueous solutions include, but are not limited to physiologically compatible buffers such as Hanks solution, Ringer’s solution, or physiological (e.g., 0.9% isotonic) saline buffer and/or in certain emulsion formulations. The solution(s) can contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. These compositions may be sterilized by conventional, well-known sterilization techniques, or may be sterile filtered. The resulting aqueous solutions may be packaged for use as is, or lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc., e.g., as described above.

In other methods, the pharmaceutical formulations containing the nanoparticle drug delivery vehicles described herein and/or one or more checkpoint inhibitors may be contacted with the target tissue by direct application of the preparation to the tissue. The application may be made by topical, “open” or “closed” procedures. By “topical” it is meant the direct application of the pharmaceutical preparation to a tissue exposed to the environment, such as the skin, oropharynx, external auditory canal, and the like. Open procedures are those procedures that include incising the skin of a patient and directly visualizing the underlying tissue to which the pharmaceutical formulations are applied. This is generally accomplished by a surgical procedure, such as a thoracotomy to access the lungs, abdominal laparotomy to access abdominal viscera, or other direct surgical approaches to the target tissue. Closed procedures are invasive procedures in which the internal target tissues are not directly visualized but accessed via inserting instruments through small wounds in the skin. For example, the preparations may be administered to the peritoneum by needle lavage. Likewise, the pharmaceutical preparations may be administered to the meninges or spinal cord by infusion during a lumbar puncture followed by appropriate positioning of the patient as commonly practiced for spinal anesthesia or metrizamide imaging of the spinal cord. Alternatively, the preparations may be administered through endoscopic devices. In certain embodiments the pharmaceutical formulations are introduced via a cannula.

In certain embodiments the pharmaceutical formulations comprising the nanoparticle drug delivery vehicles described herein and/or one or more checkpoint inhibitors are administered via inhalation (e.g., as an aerosol). Inhalation can be a particularly effective delivery route for administration to the lungs and/or to the brain. For administration by inhalation, the nanoparticle drug delivery vehicles are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator may be formulated containing a powder mix of the compound and a suitable powder base such as lactose or starch.

In certain embodiments, the nanoparticle drug delivery vehicles described herein and/or one or more checkpoint inhibitors are formulated for oral administration. For oral administration, suitable formulations can be readily formulated by combining the drug delivery vehicles with pharmaceutically acceptable carriers suitable for oral delivery well known in the art. Such carriers enable the active agent(s) described herein to be formulated as tablets, pills, dragees, caplets, lozenges, gelcaps, capsules, liquids, gels, syrups, slurries, suspensions and the like, for oral ingestion by a patient to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents; and binding agents. If desired, disintegrating agents may be added, such as the crosslinked polyvinylpyrrolidone, agar, or alginic acid or a salt thereof such as sodium alginate. If desired, solid dosage forms may be sugar-coated or enteric-coated using standard techniques. The preparation of enteric-coated particles is disclosed for example in U.S. Pat. Nos. 4,786,505 and 4,853,230.

In various embodiments the nanoparticle drug delivery vehicles described herein and/or one or more checkpoint inhibitors can be formulated in rectal or vaginal compositions such as suppositories or retention enemas, e.g., containing conventional suppository bases such as cocoa butter or other glycerides. Methods of formulating active agents for rectal or vaginal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45), amphiphilic materials such as Suppocire AP and polyglycolized glyceride, and the like). The base is selected and compounded for a desired melting/delivery profile.

The route of delivery of the nanoparticle drug delivery vehicles described herein and/or one or more checkpoint inhibitors can also affect their distribution in the body. Passive delivery of the drug delivery vehicles involves the use of various routes of administration e.g., parenterally, although other effective administration forms, such as intraarticular injection, inhalant mists, orally active formulations, transdermal iontophoresis, or suppositories are also envisioned. Each route produces differences in localization of the drug delivery vehicle.

Because dosage regimens for pharmaceutical agents are well known to medical practitioners, the amount of the liposomal pharmaceutical agent formulations and/or one or more checkpoint inhibitors that is effective or therapeutic for the treatment of a disease or condition in mammals and particularly in humans will be apparent to those skilled in the art. The optimal quantity and spacing of individual dosages of the formulations herein will be determined by the nature and extent of the condition being treated, the form, route and site of administration, and the particular patient being treated, and such optima can be determined by conventional techniques. It will also be appreciated by one of skill in the art that the optimal course of treatment, e.g., the number of doses given per day for a defined number of days, can be ascertained by those skilled in the art using conventional course of treatment determination tests.

Typically, the nanoparticle drug delivery vehicles described herein and/or pharmaceutical formations thereof described herein and/or one or more checkpoint inhibitors are used therapeutically in animals (including man) in the treatment of various cancers. In certain embodiments the drug delivery vehicles and/or pharmaceutical formations thereof described herein are particularly well suited in conditions that require: (1) repeated administrations; and/or (2) the sustained delivery of the drug in its bioactive form; and/or (3) the decreased toxicity with suitable efficacy compared with the free drug(s) in question. In various embodiments the nanoparticle drug delivery vehicles and/or pharmaceutical formations thereof are administered in a therapeutically effective dose. The term “therapeutically effective” as it pertains to the nanoparticle drug delivery vehicles described herein and formulations thereof means that GSK3 inhibitor contained therein, alone or in combination with other drugs, produces a desirable effect on the cancer. Such desirable effects include, but are not limited to slowing and/or stopping tumor growth and/or proliferation and/or slowing and/or stopping proliferation of metastatic cells, reduction in size and/or number of tumors, and/or elimination of tumor cells and/or metastatic cells, and/or prevention of recurrence of the cancer following remission.

Exact dosages will vary depending upon such factors as the particular GSK3 inhibitor and the desirable medical effect, as well as patient factors such as age, sex, general condition, and the like. Those of skill in the art can readily take these factors into account and use them to establish effective therapeutic concentrations without resort to undue experimentation.

For administration to humans (or to non-human mammals) in the curative, remissive, retardive, or prophylactic treatment of diseases the prescribing physician will ultimately determine the appropriate dosage of the drug(s) for a given human (or non-human) subject, and this can be expected to vary according to the age, weight, and response of the individual as well as the nature and severity of the patient’s disease. In certain embodiments the dosage of the drug provided by the nanoparticle drug delivery vehicles can be approximately equal to that employed for the free drug. However as noted above, the nanoparticle drug delivery vehicles described herein can significantly reduce the toxicity of the drug(s) administered thereby and significantly increase a therapeutic window. Accordingly, in some cases dosages in excess of those prescribed for the free drug(s) will be utilized.

In certain embodiments, the dose of each of the drug(s) (e.g., camptothecin analog(s)) and/or checkpoint inhibitors administered at a particular time point will be in the range from about 1 to about 1,000 mg/m²/day, or to about 800 mg/m²/day, or to about 600 mg/m²/day, or to about 400 mg/m²/day. For example, in certain embodiments a dosage (dosage regiment) is utilized that provides a range from about 1 to about 350 mg/m²/day, 1 to about 300 mg/m²/day, 1 to about 250 mg/m²/day, 1 to about 200 mg/m²/day, 1 to about 150 mg/m²/day, 1 to about 100 mg/m²/day, from about 5 to about 80 mg/m²/day, from about 5 to about 70 mg/m²/day, from about 5 to about 60 mg/m²/day, from about 5 to about 50 mg/m²/day, from about 5 to about 40 mg/m²/day, from about 5 to about 20 mg/m²/day, from about 10 to about 80 mg/m²/day, from about 10 to about 70 mg/m²/day, from about 10 to about 60 mg/m²/day, from about 10 to about 50 mg/m²/day, from about 10 to about 40 mg/m²/day, from about 10 to about 20 mg/m²/day, from about 20 to about 40 mg/m²/day, from about 20 to about 50 mg/m²/day, from about 20 to about 90 mg/m²/day, from about 30 to about 80 mg/m²/day, from about 40 to about 90 mg/m²/day, from about 40 to about 100 mg/m²/day, from about 80 to about 150 mg/m²/day, from about 80 to about 140 mg/m²/day, from about 80 to about 135 mg/m²/day, from about 80 to about 130 mg/m²/day, from about 80 to about 120 mg/m²/day, from about 85 to about 140 mg/m²/day, from about 85 to about 135 mg/m²/day, from about 85 to about 135 mg/m²/day, from about 85 to about 130 mg/m²/day, or from about 85 to about 120 mg/m²/day. In certain embodiments the does administered at a particular time point may also be about 130 mg/m²/day, about 120 mg/m²/day, about 100 mg/m²/day, about 90 mg/m²/day, about 85 mg/m²/day, about 80 mg/m²/day, about 70 mg/m²/day, about 60 mg/m²/day, about 50 mg/m²/day, about 40 mg/m²/day, about 30 mg/m²/day, about 20 mg/m²/day, about 15 mg/m²/day, or about 10 mg/m²/day.

In certain embodiments, the dose administered may be higher or lower than the dose ranges described herein, depending upon, among other factors, the bioavailability of the composition, the tolerance of the individual to adverse side effects, the mode of administration and various factors discussed above. Dosage amount and interval may be adjusted individually to provide plasma levels of the composition that are sufficient to maintain therapeutic effect, according to the judgment of the prescribing physician. Skilled artisans will be able to optimize effective local dosages without undue experimentation in view of the teaching provided herein.

Multiple doses (e.g., continuous or bolus) of the compositions as described herein may also be administered to individuals in need thereof of the course of hours, days, weeks, or months. For example, but not limited to, 1, 2, 3, 4, 5, or 6 times daily, every other day, every 10 days, weekly, monthly, twice weekly, three times a week, twice monthly, three times a month, four times a month, five times a month, every other month, every third month, every fourth month, etc.

### Methods of Treatment

In various embodiments methods of treatment using the nanoparticle drug delivery vehicles described herein and/or pharmaceutical formulation(s) comprising the nanoparticle drug delivery vehicles described herein in combination with one or more checkpoint inhibitors are provided. In certain embodiments the method(s) comprise a method of treating a cancer. In certain embodiments the method can comprise administering to a subject in need thereof an effective amount of one or more checkpoint inhibitors and a nanoparticle drug delivery vehicle described herein containing one or more camptothecin analogs (e.g., IRIN), and/or a pharmaceutical formulation comprising the nanoparticle drug delivery vehicles.

In certain embodiments the nanoparticle drug delivery vehicles described herein the combination of a nanoparticle drug delivery vehicle containing one or more camptothecin analogs in combination with one or more checkpoint inhibitors is a primary therapy in a chemotherapeutic regimen. In certain embodiments the combination of a nanoparticle drug delivery vehicle containing one or more camptothecin analogs in combination with one or more checkpoint inhibitors is a component in an adjunct therapy in addition to chemotherapy using one or more other chemotherapeutic agents, and/or surgical resection of a tumor mass, and/or radiotherapy.

In certain embodiments the nanoparticle drug delivery vehicle (containing one or more camptothecin analogs) and one or more checkpoint inhibitors are components in a multi-drug chemotherapeutic regimen. In certain embodiments the multi-drug chemotherapeutic regimen additionally comprises at least two drugs selected from the group consisting of irinotecan (IRIN) (or other camptothecin analog provided by the nanoparticle drug delivery vehicles described herein), one or more checkpoint inhibitors, oxaliplatin (OX), 5-fluorouracil (5-FU), and leucovorin (LV

In various embodiments the nanoparticle drug delivery vehicles and/or pharmaceutical formulation(s) thereof described herein in combination with one or more checkpoint inhibitors are effective for treating any of a variety of cancers. In certain embodiments the cancer is pancreatic ductal adenocarcinoma (PDAC). In certain embodiments the cancer is a cancer selected from the group consisting of acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, glioblastoma, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm’s tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, chronic myeloid leukemia (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm’s tumor.

In certain embodiments the nanoparticle drug delivery vehicles described herein are not conjugated to an iRGD peptide and the drug delivery vehicles are administered in conjunction with an iRGD peptide (e.g., the drug delivery vehicle and the iRGD peptide are co-administered as separate formulations).

In various embodiments of these treatment methods, the nanoparticle drug delivery vehicles described herein and/or pharmaceutical formulation is administered via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition. In certain embodiments the drug delivery vehicles and/or pharmaceutical formulations thereof are administered as an injection, from an IV drip bag, or via a drug-delivery cannula. In various embodiments the subject is a human and in other embodiments the subject is a non-human mammal.

### Kits

In certain embodiments, kits are provided containing reagents for the practice of any of the methods described herein. In certain embodiments the kit comprises a container containing a drug delivery vehicle described herein where the drug delivery vehicle contains at least one or more camptothecin analogs. In certain embodiments, the drug delivery vehicle additionally contains one or more autophagy inhibitors.

In certain embodiments, the kits additionally comprise a container containing one or more checkpoint inhibitors as described herein.

Additionally, in certain embodiments, the kits can include instructional materials disclosing the means of the use of the nanoparticle drug delivery vehicles in combination with checkpoint inhibitors as described herein as a cancer therapeutic.

In addition, the kits optionally include labeling and/or instructional materials providing directions (e.g., protocols) for the use of the materials described herein, e.g., alone or in combination for the treatment of various cancers. Instructional materials can also include recommended dosages, description(s) of counterindications, and the like.

While the instructional materials in the various kits typically comprise written or printed materials they are not limited to such. Any medium capable of storing such instructions and communicating them to an end user is contemplated by this invention. Such media include but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Combination Chemo-Immunotherapy for Pancreatic Cancer Using the Immunogenic

### Effects of an Irinotecan Silicasome Nanocarrier Plus Anti-PD-1

We have previously demonstrated improved irinotecan (IRIN) delivery by mesoporous silica nanoparticles (MSNP), coated with a lipid bilayer, in a robust treatmentresistant Kras-induced pancreatic cancer (KPC) model^{[16]}, derived from a spontaneous Kras^{LSL-G12D/+}; Trp53^{LSL-R172H/+}; Pdx-1-Cre (KPC) tumor^{[17]}. We also refer to the MSNPs carrier as a “silicasome”. While effective for improving the chemotherapy response in the orthotopic KPC model, the experimentation did not involve a study of the immunogenic effects of IRIN, which until now have been labeled as “non-determined”^{[12e].} The possibility that an immunogenic effect must be entertained emerged from studies on the KPC cell line, which demonstrated that IRIN could induce CRT expression and HMGB1 release that curiously was combined with autophagy inhibition instead of autophagy flux simulation, as seen in the conventional ICD model^{[12f,} ^{14a]}. This prompted extensive investigation into the biological effects of IRIN to explain its immunogenic effects, including whether these responses could be used to instigate for initiating immunotherapy in vivo. Our results will delineate that an additional dimension of the IRIN treatment response involves the neutralizing effect of the free or encapsulated drug on lysosomal pH, which is associated with autophagy inhibition and triggering of an ICD response that is dependent on a primary endoplasmic reticulum (ER) stress response. Moreover, we also show that the autophagy inhibition is associated with increased PD-L1 expression on KPC cells. These findings provided the basis for studying the chemo-immunotherapy response to IRIN-delivery silicasomes in an orthotopic KPC model. We also asked whether the effect could be combined with the delivery of anti-PD-1 antibodies.

### Results

### IRIN Leads to Lysosomal Alkalization, Which is Linked to Autophagy Inhibition and PD-L1 Overexpression in KPC Cells

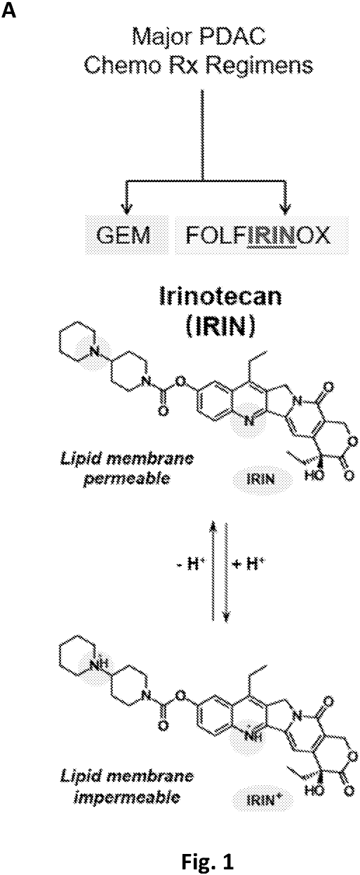

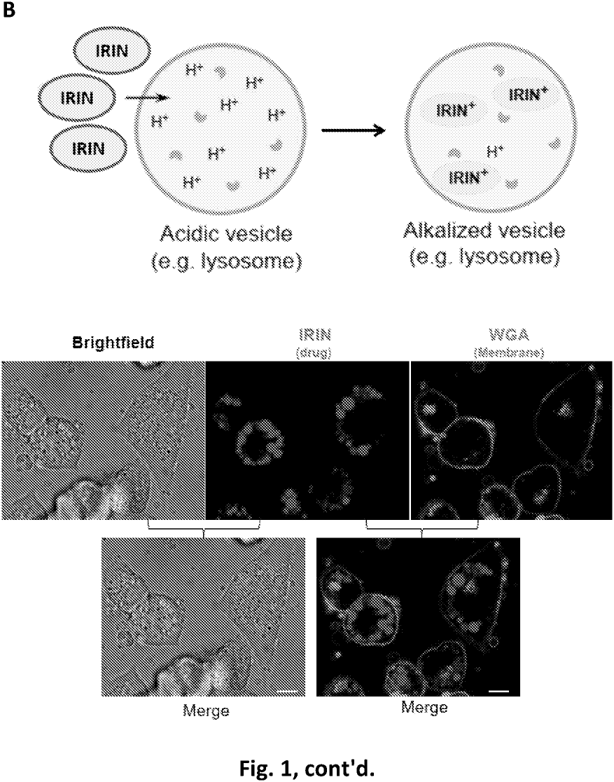

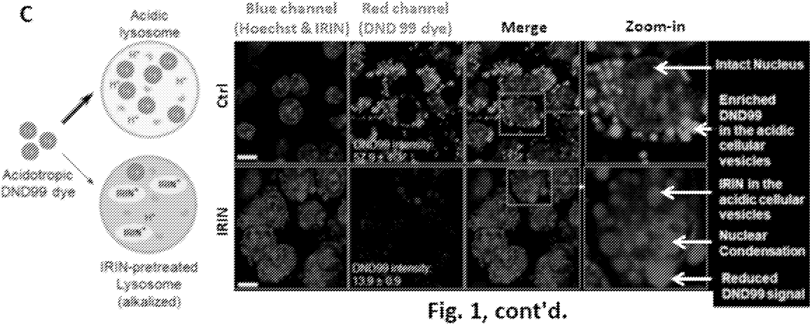

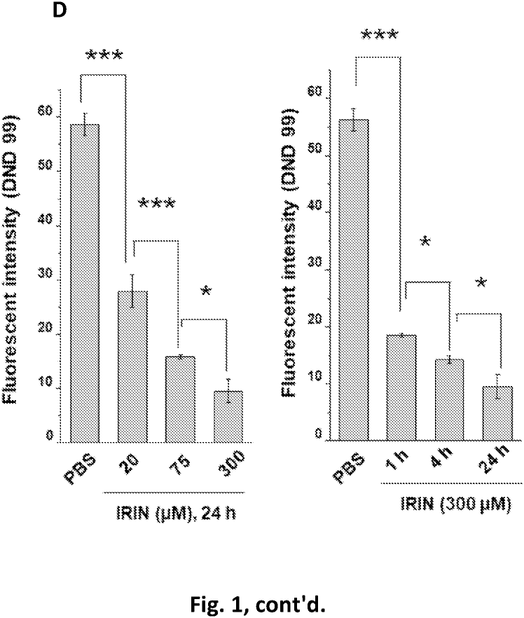

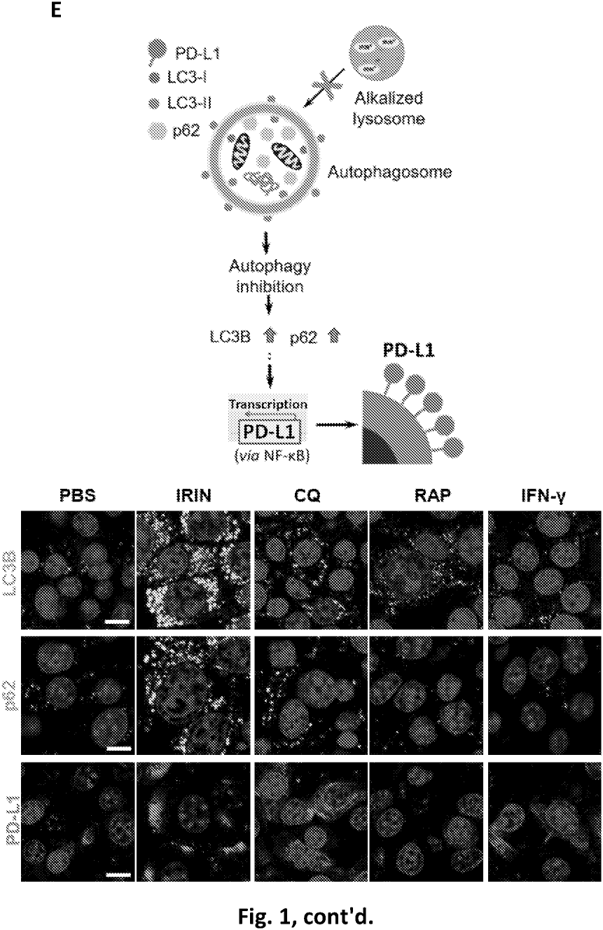

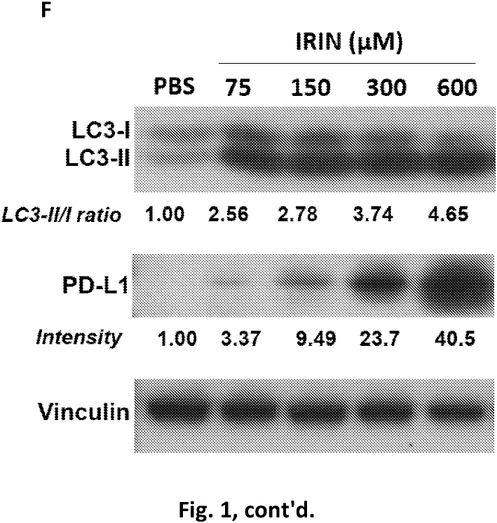

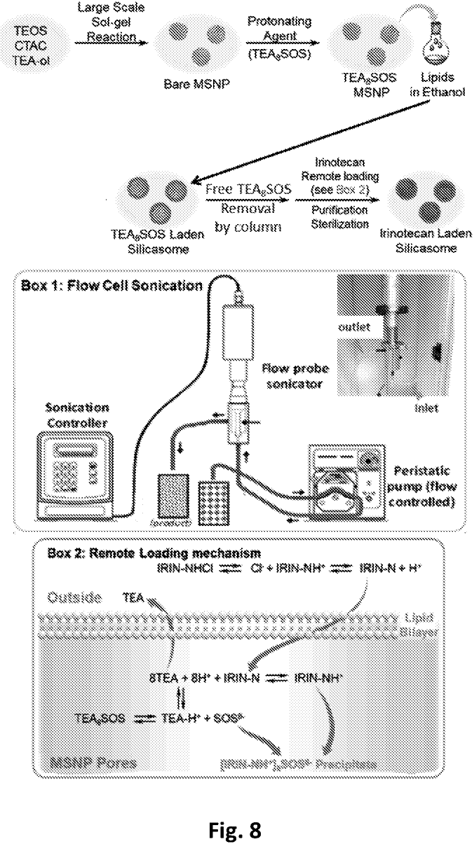

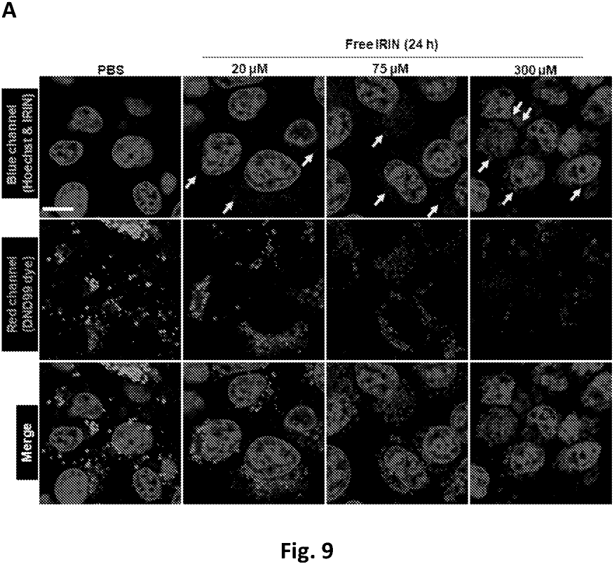

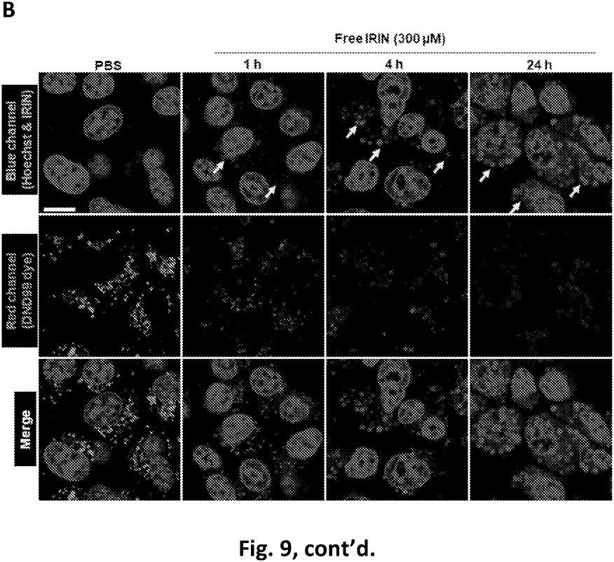

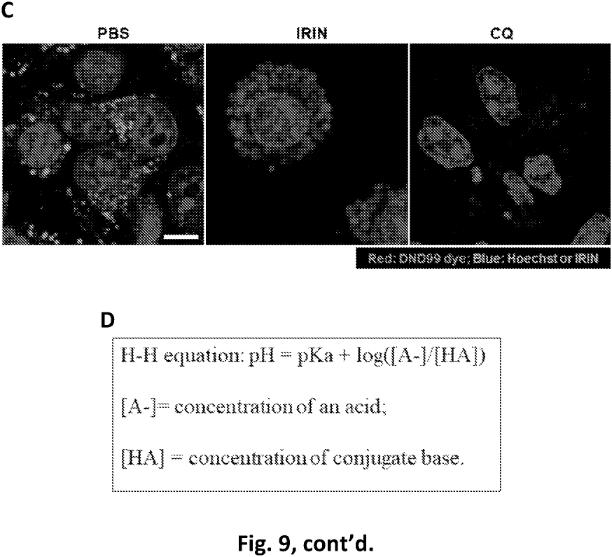

IRIN is a weak base (pKa = 8.1) that can be readily protonated in an acidic environment^{[18]}. In fact, we make use of this property for remote loading of IRIN into the silicasome carrier (FIG. 8, Box 2). This is premised on the principle that the nonprotonated, amphiphilic drug, is capable of diffusing across the coated lipid bilayer, where its protonation by an encapsulated trapping agent (triethylammonium sucrose octasulfate) leads to the generation of hydrophilic IRIN, which is incapable of back-diffusion across the lipid bilayer^{[16a]}. The ability to achieve drug compartmentalization across an artificial lipid bilayer also prompted us to ask whether IRIN can cross cell membranes and become entrapped in acidifying cellular compartments? Utilizing the fluorescent (blue) properties of IRIN, it was possible to demonstrate in a confocal study that the drug was taken up in a vesicular compartment that localizes closely to the wheat germ agglutinin (WGA) stained surface membrane in KPC cells (FIG. 1,panel B). To confirm that this constitutes an acidifying compartment, a weak-basic acidotropic dye, Lysotracker™ Red DND 99, was used to determine the impact of IRIN on its red fluorescent signal in a confocal microscopy experiment^{[19]} (FIG. 1, panel C). Thus, while DND 99 could be seen to localize in the lysosomal compartment of untreated KPC cells (FIG. 1,panel C, upper right panel), there was a sharp reduction in the dye’s red fluorescence intensity in cells that were prior treated with IRIN (FIG. 1, panel C, lower right panel). Image J software was used for quantifying the shift in DND 99 fluorescence intensity, allowing us to demonstrate that IRIN treatment could significantly reduce the relative staining intensity from 57.9 ± 1.3 to 13.9 ± 0.9 in KPC cells. This allowed the blue fluorescence of the drug to be observed, in addition to the appearance of nuclear condensation in the dying cells (FIG. 1, panel C). Noteworthy, that the IRIN treatment effect was both dose- (FIG. 1, panel D, left) and time-dependent (FIG. 1, panel D, right); the corresponding confocal images appear in FIG. 9, panels A and B. The alkalizing effect of IRIN was duplicated by chloroquine (CQ), which has a well-known buffering effect on the acidic lysosomal pH in cells (FIG. 9, panel C)^{[20]}. The effect of the encapsulated drug will be discussed herein.

In addition to the role of the acidic pH in the destruction of lysosomal content, lysosomal acidification is also important for the fusion of this organelle with the autophagosome^{[20b, 21]}. Thus, we were interested to determine if IRIN can interfere in autophagy flux, as previously demonstrated, through the use of CQ or gene deletion of the proton-generating V-ATPase subunit of the lysosome^{[22]}. The methodology for demonstrating autophagy inhibition is to show the presence of LC3B complexes, which are involved in the formation of the autophagosome, as well as the accumulation of the sensor protein, p62/SQSTM1, which detects toxic cellular waste products and is removed and destroyed with the waste products in the lysosome^{[20a, 21a, 23]}. This was accomplished by the performance of confocal microscopy to demonstrate the intracellular appearance of immunofluorescence (IF) stained LC3B and p62/SQSTM1 complexes, as shown in FIG. 1, panel E. Confocal viewing demonstrated that IRIN treatment leads to the contemporaneous appearance of fluorescent LC3B puncta as well as p62 protein complexes in KPC cells (FIG. 1, panel E, right panel). In order to confirm that dual fluorescent staining discerns autophagy inhibition, we also used CQ to demonstrate the appearance of similar immunofluorescence features (FIG. 1, panel E). In contrast, rapamycin (RAP), which functions as an autophagy inducer, resulted in LC3B assembly without p62 accumulation[^{20a,} ^{21a, 23]}. The assembly of LC3B complexes were confirmed in an immunoblotting assay, which demonstrated a dose-dependent increase in the ratio of the LC3-II vs. LC3-I expression during IRIN treatment (FIG. 1, panel F).

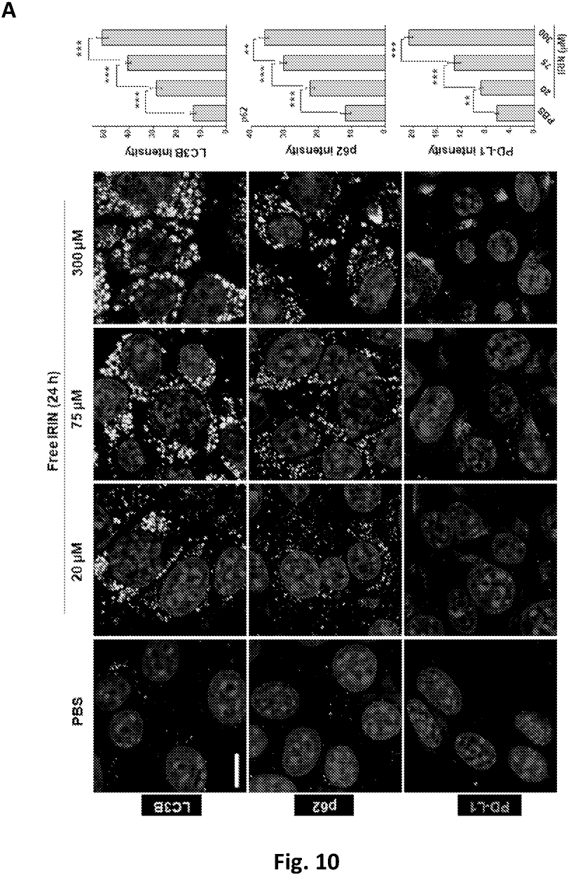

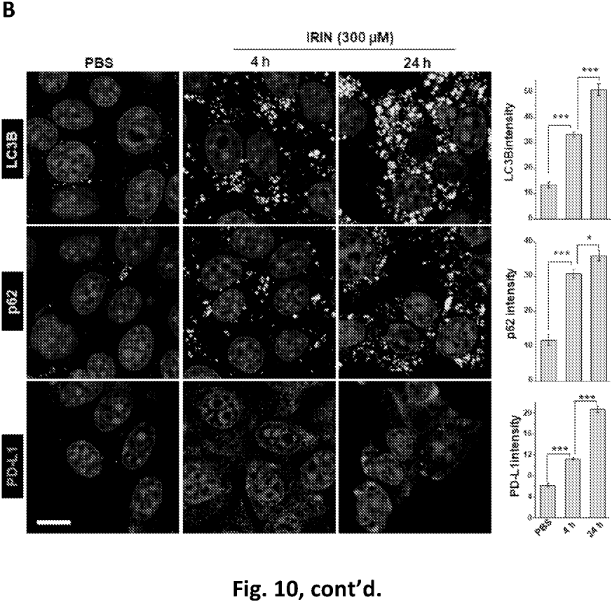

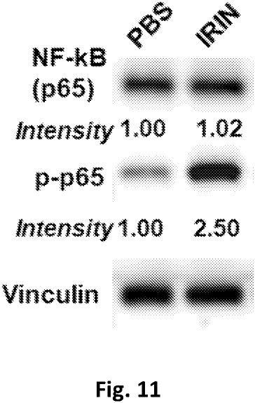

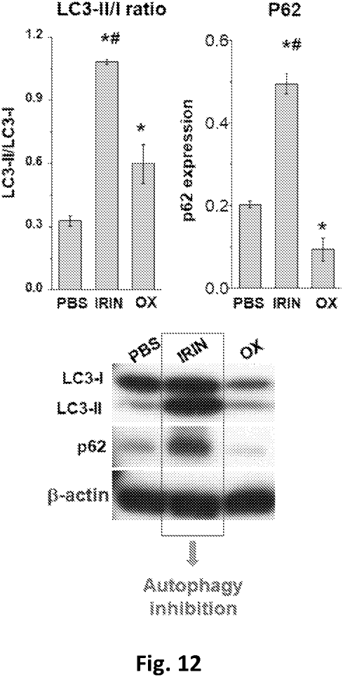

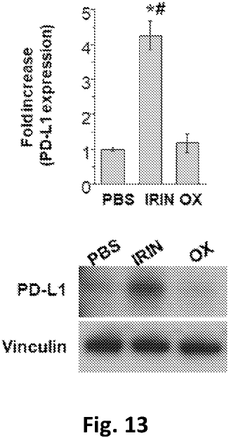

Of specific importance to the objective of addressing IRIN immunogenicity, it has recently been demonstrated that the accumulation of p62/SQSTM1 during pharmacological disruption of autophagy can trigger PD-L1 expression in gastric cancer cells^{[23]}. Not only did we demonstrate treatment can induce robust cell surface expression of PD-L1 in KPC cells during the performance of confocal microscopy (FIG. 1, panel E, bottom right panel), but also observed the same effect during immunoblotting (FIG. 1, panel F). These results were in in agreement with the effect of CQ or cellular treatment with IFN-γ, a robust inducer of the PD-L1 promoter^{[24]} (FIG. 1, panel E, bottom right panel). Noteworthy, these responses were dose- and time-dependent, as shown in the immunoblotting (FIG. 1, panel F) and confocal experiments (FIG. 10, panels A and B). A possible mechanism to explain PD-L1 expression by p62/SQSTM1 in gastric cancer has been the demonstration of NF-κB activation, which impacts the PD-L1 promoter^{[23]}. We confirmed that that IRIN treatment can induce the phosphorylation of the p65 (p-p65) subunit of NF-κB in KPC cells (FIG. 11). In contrast to IRIN, oxaliplatin (OX), a potent non-basic PDAC chemo agent induced autophagy (FIG. 12) but failed to upregulate PD-L1 expression in KPC cells (FIG. 13).

### The Autophagosomal Inhibitory Effect of IRIN is Accompanied by Endoplasmic Reticulum (ER) Stress, an Inducer of Immunogenic Cell Death Pathways

The ablation of autophagy has been linked to the generation of ER stress and the generation of immunogenic cell death in cancer cells during hypericin-mediated photodynamic therapy (Hyp-PDT)^{[25]}. Not only is ER stress a common feature of ER stress, but has also been used to distinguish between an immunogenic cell death pathway (“Type I” ICD response) that primarily targets the nucleus, with secondary impact on the ER, as compared to a “Type II” pathway in which ER stress is the primary event that secondarily leads to cell death and nuclear involvement^{[13, 14b, 26]}. Thus, while most chemotherapeutics engaged in ICD effects, e.g. DOX and OX, have been characterized as Type I ICD inducers, a few novel platinum agents (e.g., Pt-N-heterocyclic carbene) and physicochemical stimuli, such as Hyp-PDT, primarily induce ER stress with secondary effects on nuclear damage and apoptosis ^{[12a, 12c-e, 27]}. The basis of the hypericin-induced effect on the ER has been shown to involve reactive oxygen species (ROS) production that leads to ER-associated proteotoxicity, a.k.a. an unfolded protein response^{[13, 14b, 26]}. We focused on unfolded protein response, which leads to the phosphorylation of the eukaryotic initiation factor (eIF2α) that is responsible for transcriptional activation of the CCAAT-enhancer-binding protein homologous (CHOP) protein ^{[13, 28]} (FIG. 9, panel A, left panel). CHOP, in turn, is capable of inducing apoptotic cell death through the generation of immunological danger signals that promote antitumor immunity^{[13, 28].}

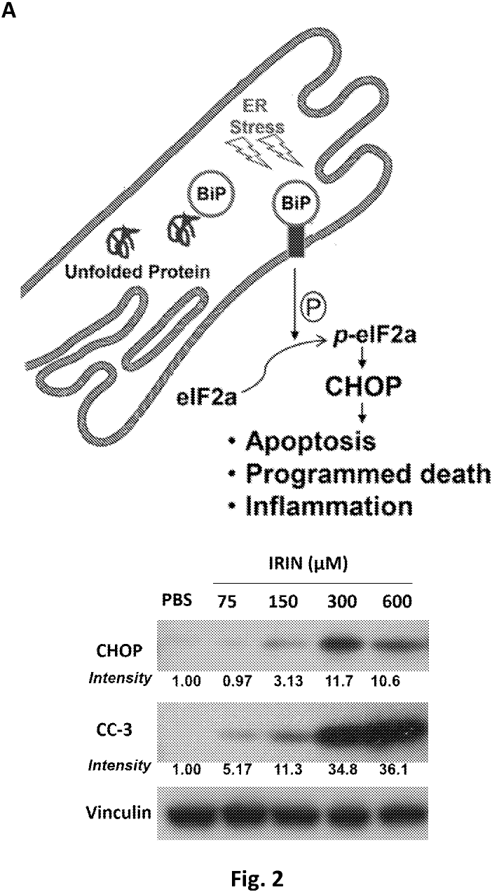

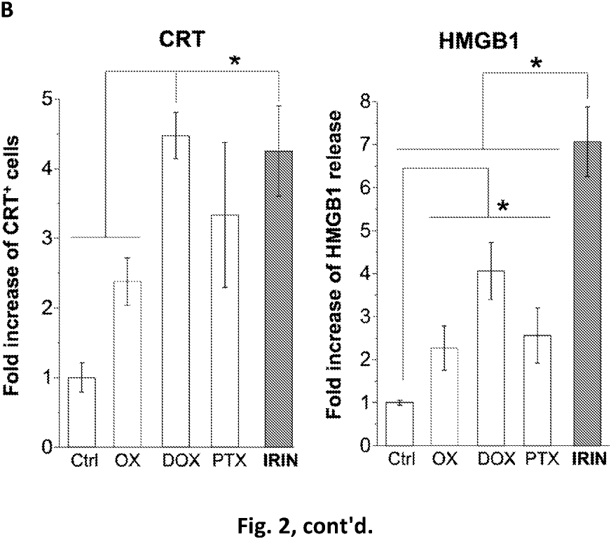

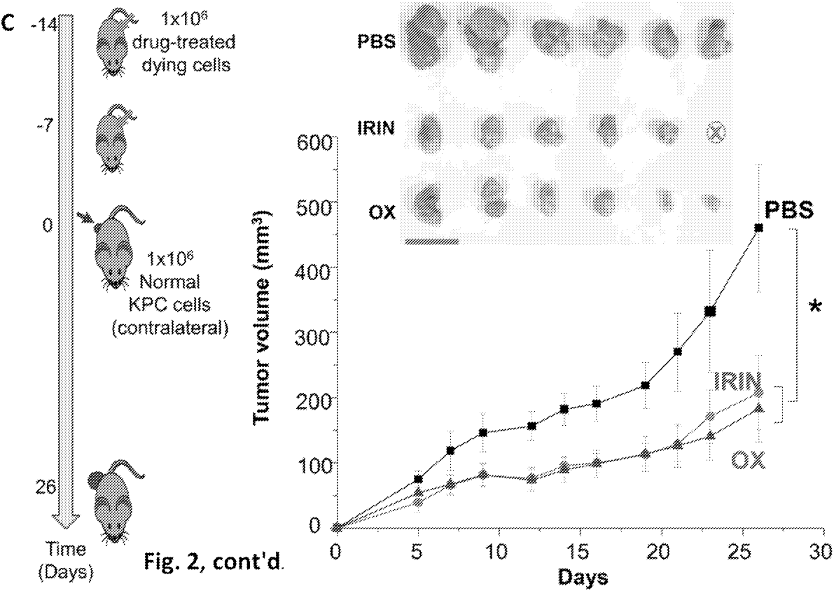

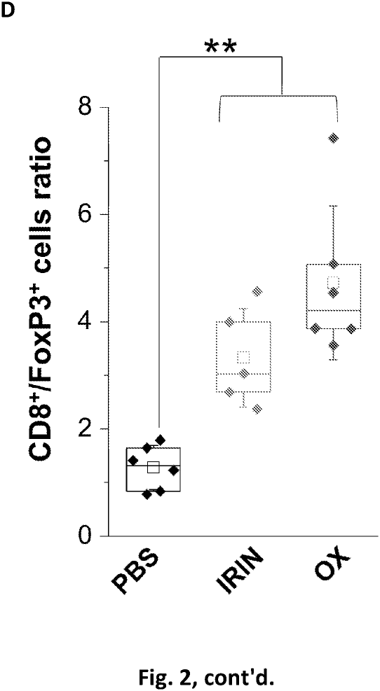

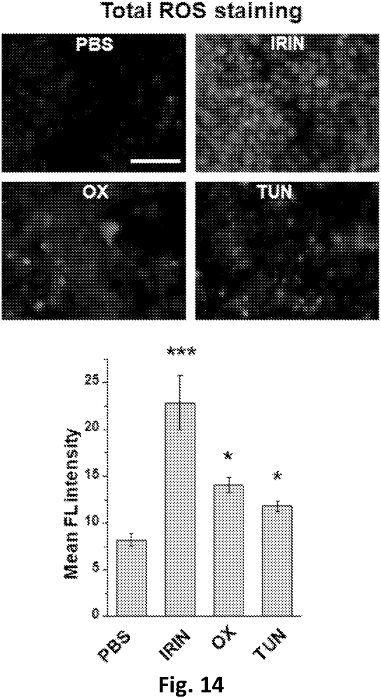

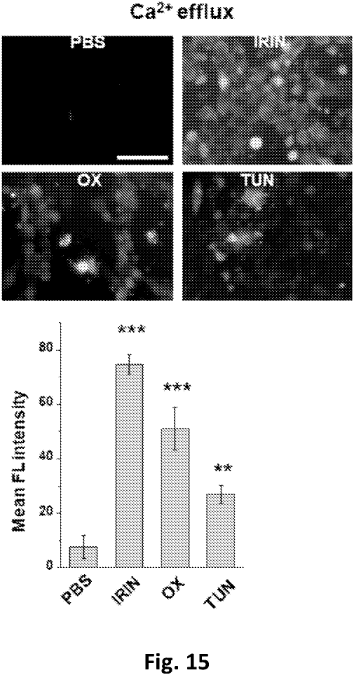

To determine the impact of IRIN on CHOP expression in KPC cells, immunoblotting analysis was used to show a sizable and dose-dependent increase in CHOP expression (FIG. 2, panel A, right panel). Moreover, this response was accompanied by increased expression of cleaved caspase-3 (CC-3), indicative of a linked apoptosis event. In order to assess the involvement of ROS generation, fluorescence microscopy was used to assess the impact of IRIN on “total ROS” production in KPC cells, using an Abcam kit^{[29]}. OX, a Type I ICD inducer, as well tunicamycin, an ER stress generating antibiotic, served as controls. (FIG. 14). The data showed robust generation ROS production in IRIN treated KPC cells (FIG. 14). It has previously been shown that the link between ROS production, ER stress and cell death involves the triggering of intracellular Ca²⁺ flux^{[28a,} ^{28c, 28d, 30]}. In order to assess intracellular Ca²⁺ release, confocal microscopy was used to perform a Fluo-4 AM assay^{[31]} in KPC cells. This demonstrated a response profile similar to ROS production (FIG. 15). All considered, these data indicate that IRIN exerts a robust ER stress response in KPC cells, which distinguishes it from the ICD effect of OX.

### IRIN Can Induce an Immunogenic Response in KPC Cells That Can Lead to a Successful Vaccination Outcome in vivo

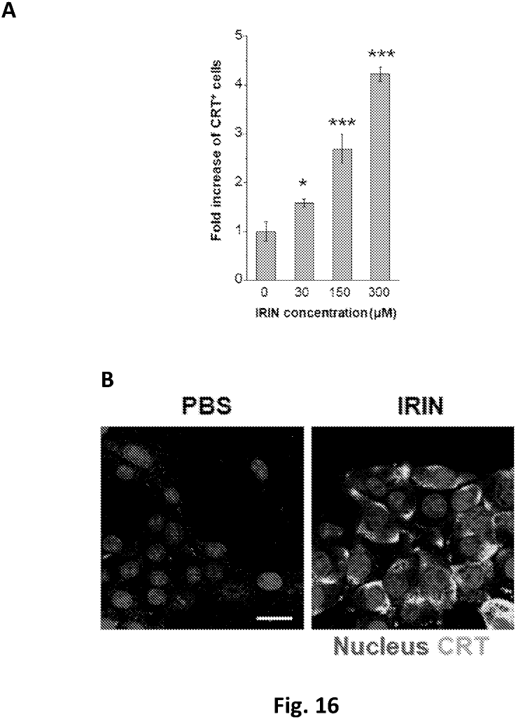

ICD, including in response to ER stimuli, is characterized by the induction of CRT translocation to the dying tumor cell surface, where it serves as an “eat-me” signal for tumor cell antigen presentation by dendritic cells^{[13]}. Cell death is also associated with the release of the chromatin protein, HMGB1, from the damaged cell nuclei ^{[13]}. IRIN was compared to OX, DOX and PTX in CRT and HGMB1 assays in KPC cells^{[12a, 14b, 27]}. Utilizing flow cytometry to assess CRT expression on the KPC surface and an ELISA for HMGB1 release in the cellular supernatant, it was possible to demonstrate that IRIN is a strong inducer of both responses (FIG. 2, panel B). Moreover, we also confirmed that the CRT response was dose-dependent (FIG. 16, panels A and B). Extracellular ATP release is often described as the third component of a typical type I ICD response^{[14b, 25b]}. However, it was of interest that we could not demonstrate an increase in ATP release by IRIN, which is in agreement with the induction of a Type II response by Hyp-PDT^{[25]} and previous demonstration that autophagy inhibition is accompanied by ATP consumption^{[32]}. Thus, in order to definitively demonstrate that IRIN induces an immunogenic cell response, we made use of an in vivo vaccination experiment as the gold standard for demonstrating assess the response outcome, as per the Consensus Guidelines for detection of ICD^{[33]}.

A vaccination experiment was performed to determine if the subcutaneous injection of dying KPC cells into the flank of syngeneic B6129SF1/J mice on two occasions could impact the growth of live tumor cells injected on the opposite flank (FIG. 2, panel C, left panel)^{[12c, 12f]}. A comparison of the vaccination response to KPC cells, treated with 300 µM IRIN or 500 µM OX, demonstrated that the generation of cell death by IRIN was comparable to the effect of OX, both of which improved the shrinkage of the tumor on the opposite flank significantly (p <0.05) compared to PBS control (FIG. 2, panel C, right panel). Moreover, tumor harvesting on day 26, followed by collection of bright field pictures, confirmed the growth inhibitory effect of the chemo agents, including total tumor disappearance in one IRIN-treated animal (right panel). The harvested tumor tissues were also used to conduct immunohistochemistry (IHC) analysis for the expression of the CD8 marker for cytotoxic T-cells and FoxP3⁺ for Treg cells (FIG. 17). This showed that while there was a slight increase in CD8⁺ staining number in response to OX (but not IRIN), both agents significantly (p <0.01) increased CD8⁺/Treg cells ratio in the quantitative response assessment (FIG. 2, panel D).

### Encapsulated IRIN Induces Lysosomal Alkalization, Autophagy Inhibition and PD-L1 Expression in PDAC Cells

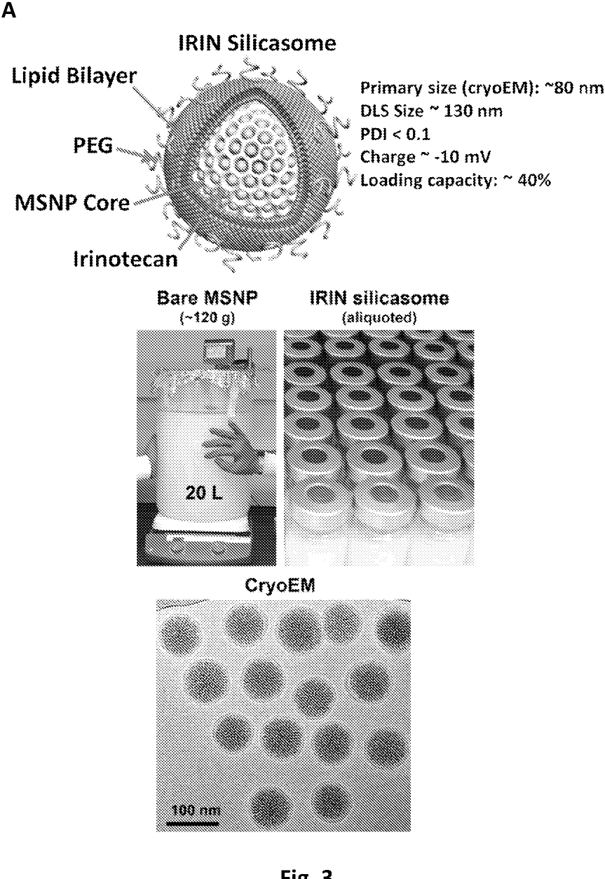

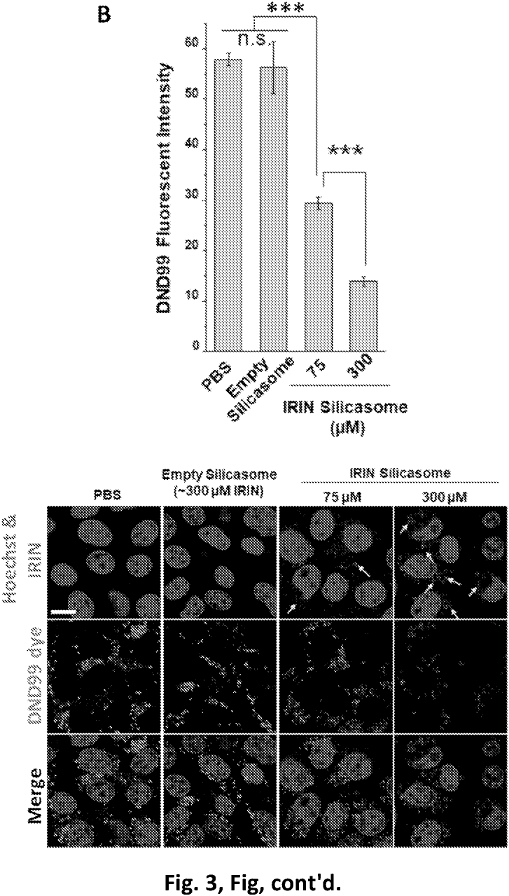

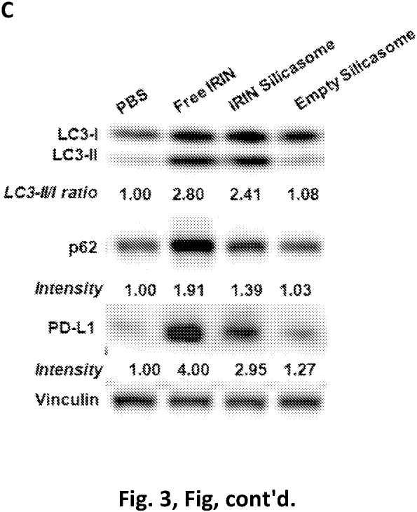

Although free IRIN is quite effective for triggering the series of linked effects whereby lysosomal alkalization, autophagy inhibition and the generation of immunogenic effects can be accomplished in KPC cells, the in vivo efficacy of the drug is considerably impaired due to the desmoplastic PDAC stroma and interference in vascular access ^{[34]} . For this reason, a liposomal carrier, Onivyde, to overcome the IRIN delivery problem and to reduce the serious side effects resulting from systemic drug administration^{[35]}. While effective, IRIN leakage from the liposome is still responsible for significant side effects, leading to receiving a black box warning from the FDA^{[3]}. To further improve IRIN delivery and toxicity reduction, we have previously established the IRIN silicasome that have the advantage of improved stability of the lipid bilayer, decreased systemic leakage and toxicity and improved drug loading compared to the liposome ^{[16c]}. A new batch of the silicasome formulation was synthesized under GLP conditions, and an aliquot was used to perform physicochemical characterization of the nanocarrier, as demonstrated in FIG. 3, panel A^{[16c]}. This demonstrated the presence of uniform particles size of ~130 nm, a slight negative charge, and a drug loading capacity of ~40 wt%.

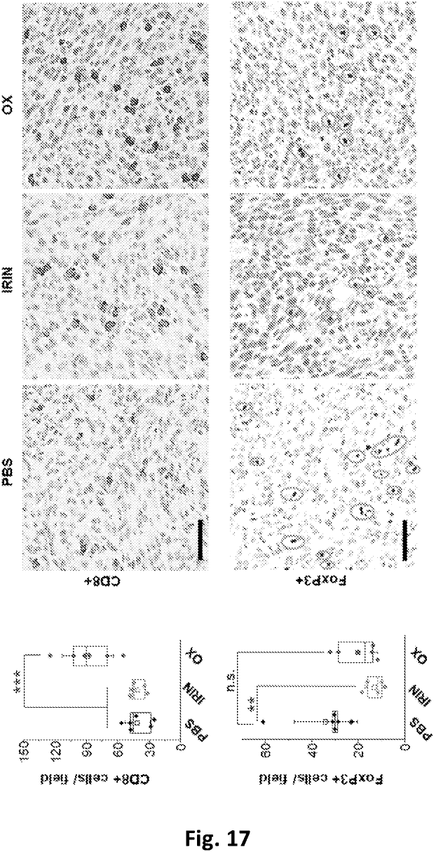

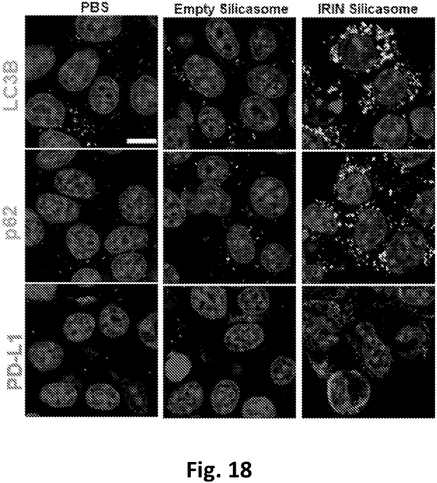

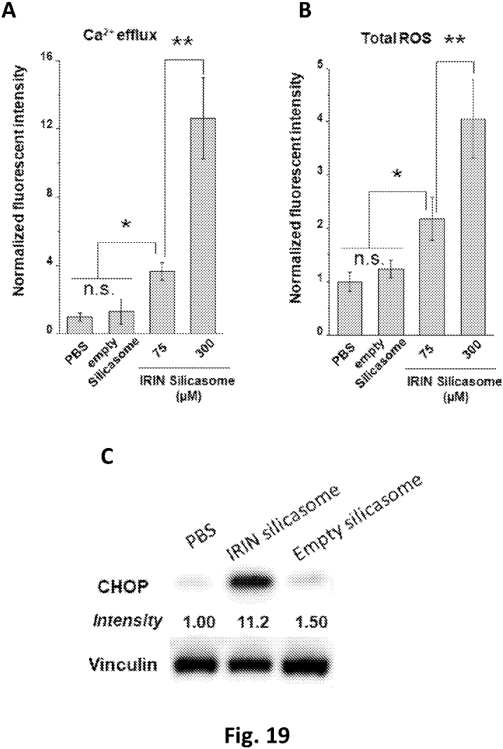

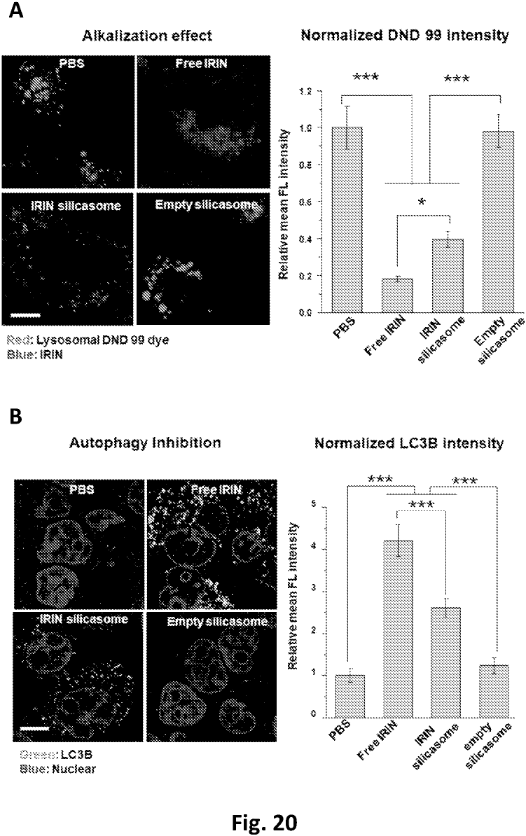

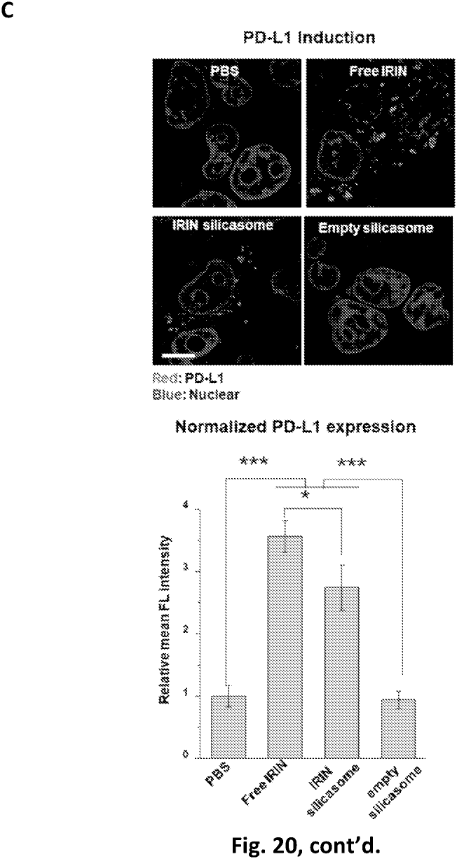

In order to assess the nanocarrier impact on lysosomal alkalization, KPC cells were incubated with the silicasomes to deliver IRIN concentrations of 75 µM and 300 µM (FIG. 3, panel B). While at the lower drug dose, the silicasome was capable of reducing the DND 99 signal by ~50% and by ~85% at 300 µM. Empty silicasomes had no effect on the alkalization. Immunoblotting assessment of LC3, p62 and PD-L1 expression confirmed the ability of the encapsulated drug to increase the LC3-II/I ratio, p62 accumulation and PD-L1 expression, similar to free drug (FIG. 3, panel C). These effects were also confirmed by confocal microscopy (FIG. 18). Moreover, the IRIN also induced intracellular Ca²⁺ flux, ROS production and CHOP expression in KPC cells in a dose-dependent fashion (FIG. 19). For example, use of the silicasome, to deliver the equivalent of a 300 µM drug dose over 48 hrs, induced an 11.2-fold increase in CHOP expression compared to the control (FIG. 19, panel C). In addition to the profiling of the murine cell line, we also confirmed the ability of IRIN silicasome, to induce lysosome alkalization, autophagy inhibition and PD-L1 expression in the frequently used human PDAC cell line, PANC-1 (FIG. 20).

### In Vivo Efficacy of the IRIN Silicasome for Inducing a Survival Effect in The Orthotopic KPC Model, Premised on Generation of an Immune Response That is Boosted by Anti-PD-1

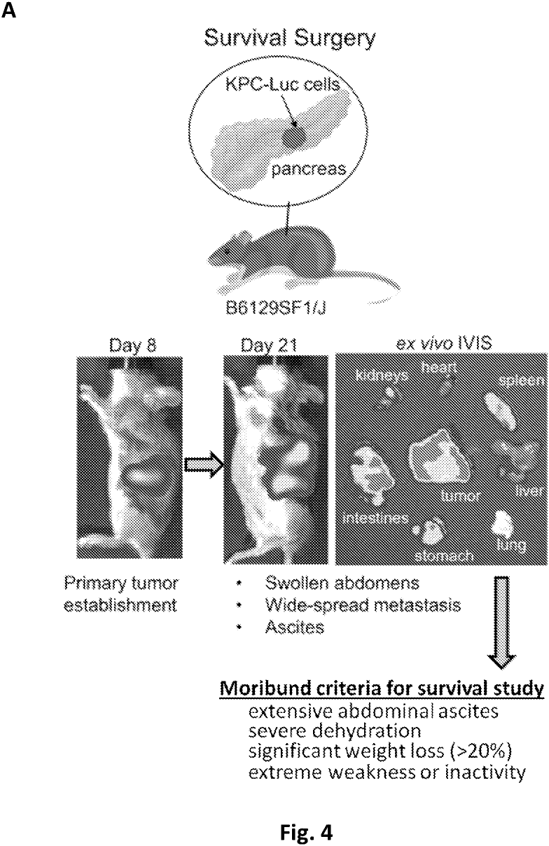

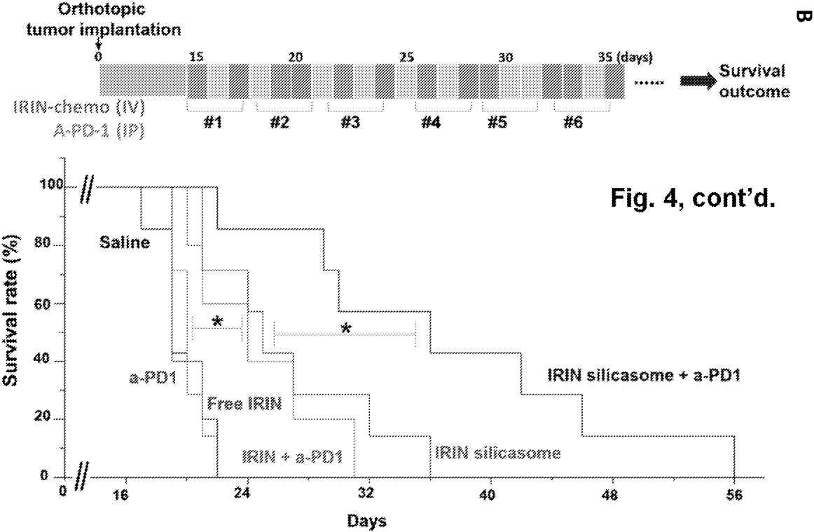

To establish the feasibility to trigger a chemo-immunotherapy response in an orthotopic KPC model in response to treatment with the IRIN-silicasome, luciferase-transfected KPC cells were implanted in the pancreatic tail of immunocompetent B6129SF1/J mice, as previously described and explained in FIG. 4, panel A ^{[16a, 16b, 17b]}. We also hypothesized that the accompanying expression of PD-L1 on KPC cells could allow the immunogenic response to be boosted by co-administering a checkpoint blocking anti-PD-1 antibody. The first was a survival experiment in which we compared the effect of free to encapsulated IRIN in the absence or presence of anti-PD-1 treatment. Orthotopic KPC tumor-bearing mice were injected IV with an IRIN dose equivalent of 40 mg/kg free or encapsulated drug every 3 or 4 days on 6 occasions (FIG. 4, panel B, upper panel, blue squares). The treatment was compared to free drug alone or in combination therapy with anti-PD-1 antibody, which was injected intraperitoneal (IP) at 100 µg/mouse 2 days after IRIN administration (pink squares). Additional controls included saline injections or mice receiving anti-PD-1 alone. Animals were monitored daily until reaching moribund status (FIG. 4, panel A) or spontaneous death. This allowed us to generate Kaplan-Meier plots, which were statistically ranked by GraphPad Prism 7.00 software^{[16, 36]}. The results demonstrated a significant improvement in the survival (p < 0.01) of silicasome-treated animals compared to free drug or anti-PD-1 alone (FIG. 4, panel B). Free drug or anti-PD-1 had no survival benefits compared to the saline control. While the combined effect of free IRIN plus anti-PD-1 was significantly improved (p < 0.05) compared to monotherapy, the best survival was obtained with the IRIN silicasome plus anti-PD-1, which was significantly better than use of the IRIN silicasome (p < 0.05) alone or use of free IRIN plus anti-PD-1 (p < 0.05) (FIG. 4, panel B). The survival data was also used to calculate “median survival time” (MST) and percent increase in life span (%ILS) vs. saline; this is a frequently used index in preclinical survival studies ^{[37]}. The MST of 36 days and %ILS of 89.5% were significantly better than other treatment groups. All considered, the data in FIG. 4 strongly support the ability of IRIN to induce an immune response that is augmented by anti-PD-1 treatment.

TABLE 5

Summary of the median survival time (MST) and percentage of increase in life span (%ILS) for each group

| | MST (d) | % ILS |
|---|---|---|
| Saline | 19 | -- |
| a-PD-1 | 20 | 5.3 |
| Free IRIN | 19 | 0.0 |
| Free IRIN + a-PD-1 | 24 | 26.3 |
| IRIN silicasome | 25 | 31.6 |
| IRIN silicasome + a-PD-1 | 36 | 89.5 |

MST: Median survival time.
%ILS: % increase in life span as calculated by [(T - C)/C] × 100

### Demonstration of an IRIN-Induced Immune Response in the Orthotopic KPC Model

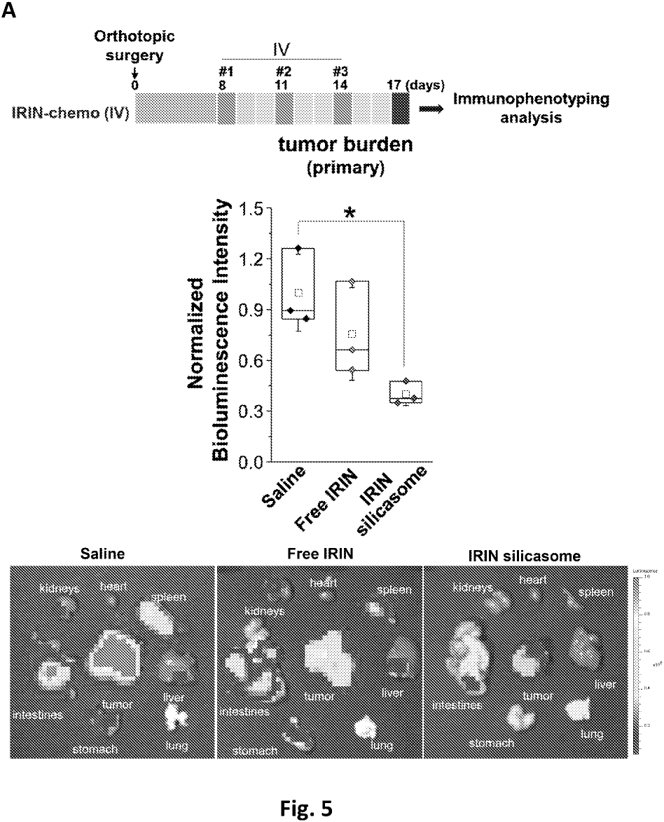

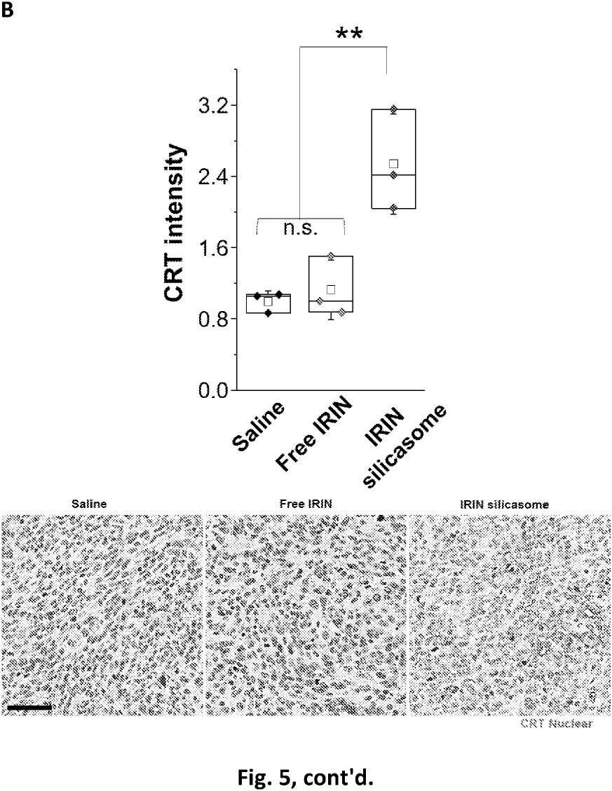

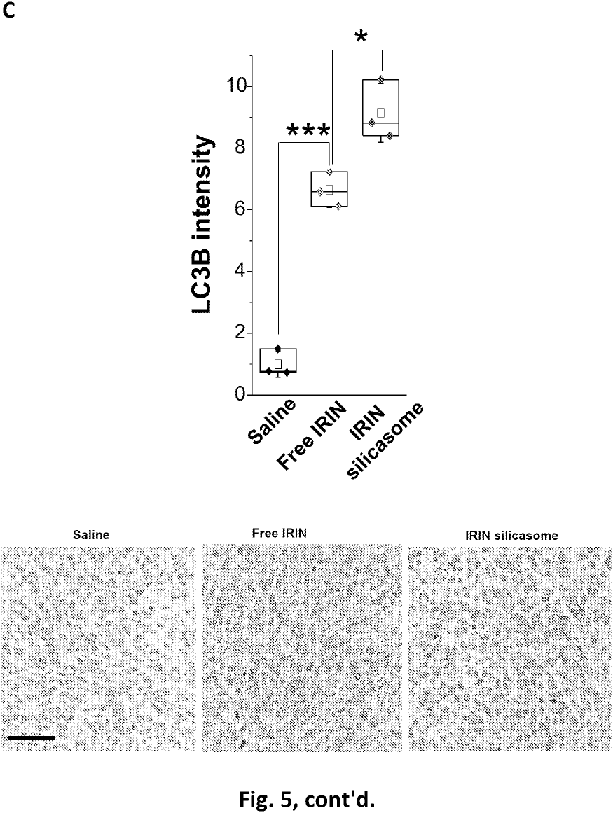

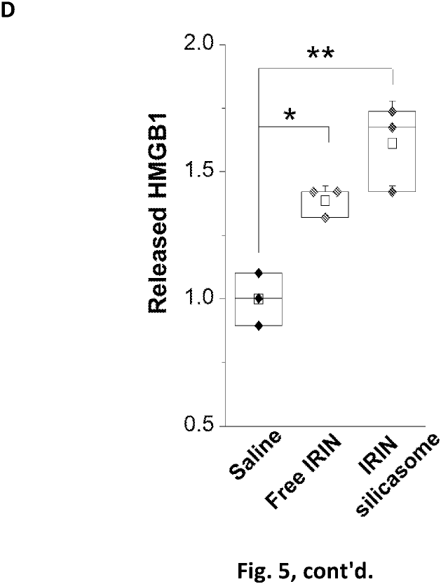

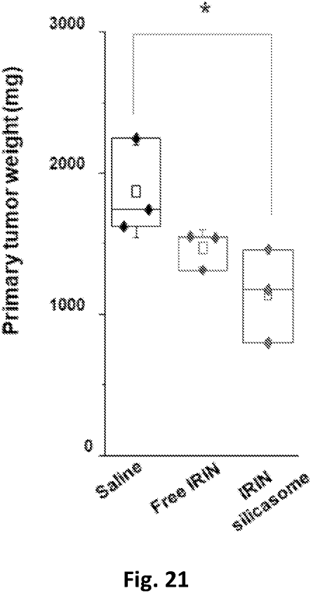

In order to assess whether the innate and cognate arms of the immune system may be involved in the response to IRIN, and efficacy experiment, coupled with the assessment of immune response markers, was carried out in animals treated with saline, free IRIN, or the IRIN silicasome (FIG. 5, panel A). The orthotopic tumor-bearing mice (n = 3 per group) received IV injection to deliver an IRIN dose equivalent of 40 mg/kg on days 8, 11, and 14. Animals were sacrificed on day 17 for tumor harvesting to perform ex vivo IVIS imaging as well as IHC analysis for immunogenic responses. From a global tumor growth perspective, quantitative assessment of bioluminescence intensity in “regions of interest” was obtained from the IVIS images, to demonstrate significant shrinkage of tumor size (p < 0.05) in animals treated with the IRIN silicasome compared to saline control (FIG. 5, panel A). While free IRIN also reduced tumor growth, the results were not statistically significant (FIG. 5, panel A). The IVIS data were further confirmed by tumor weight assessments, as outlined in FIG. 21.

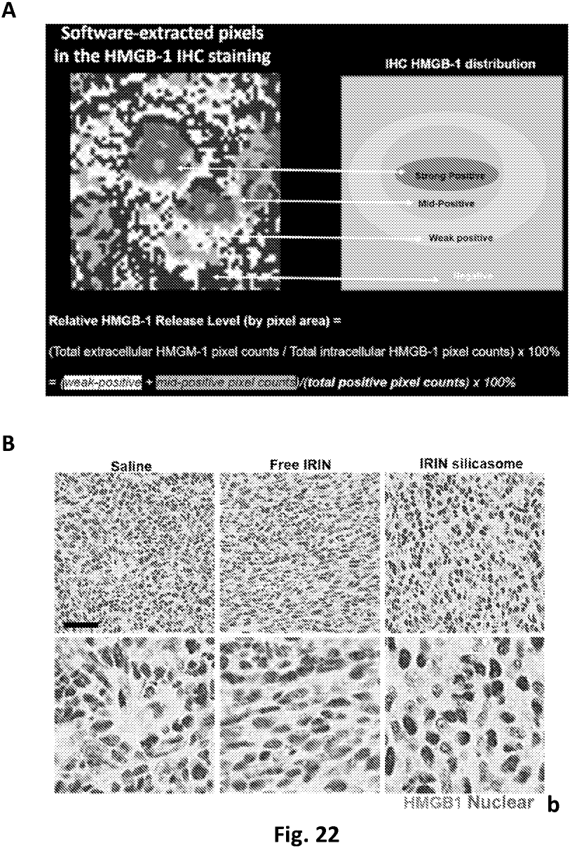

IHC analysis to assess CRT expression demonstrated that encapsulated IRIN delivery was associated with significantly higher expression of the “eat-me” biomarker (p < 0.01) compared to the staining intensity in animals treated with saline or free drug (FIG. 5, panel B). Representative images are shown on the right side (FIG. 5, panel B). IHC staining using an antibody that recognizes LC3B also demonstrated significantly increased staining intensity of the autophagy marker in tumor tissue obtained from animals treated with either free or encapsulated IRIN (p < 0.001) (FIG. 5, panel C). However, staining intensity was significantly higher for encapsulated versus free drug delivery (p < 0.05). Representative IHC images appear on the right-hand side (FIG. 5, panel C). Essentially similar results were obtained for HMGB1 staining (FIG. 5, panel D), which required the software analysis to be adapted to quantify the amount of released protein from the damaged nuclei, as explained in FIG. 22, panel A. Representative IHC images are shown in FIG. 22, panel B.

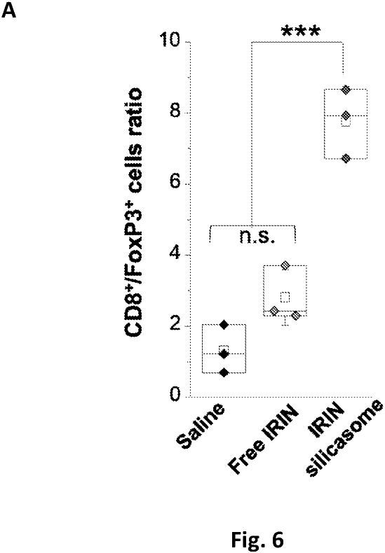

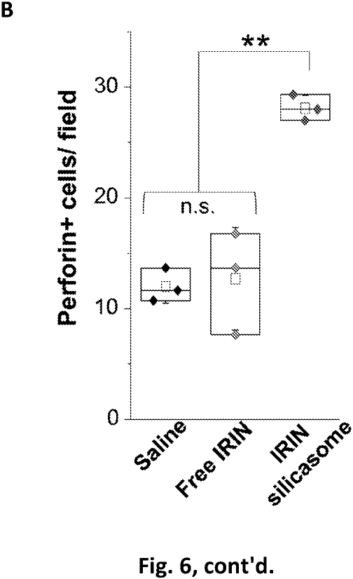

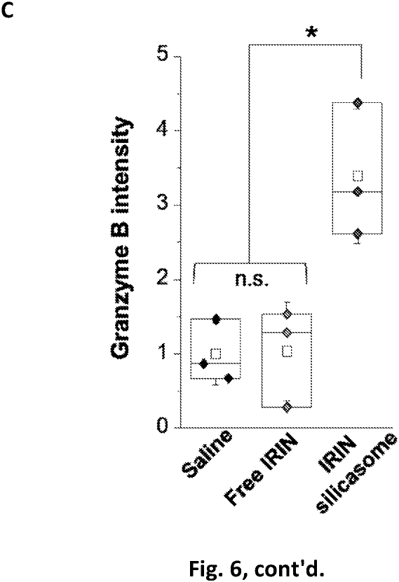

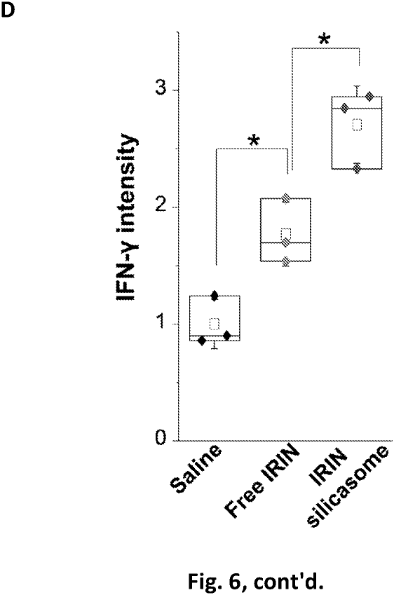

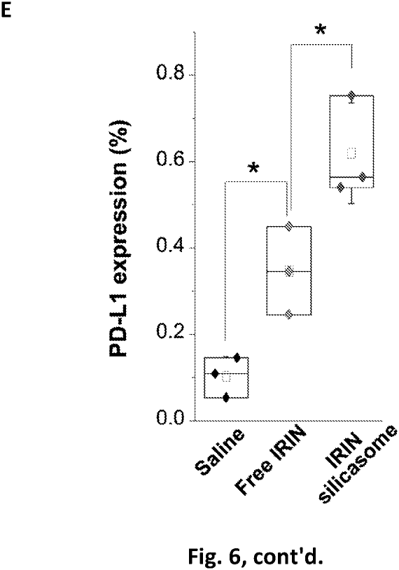

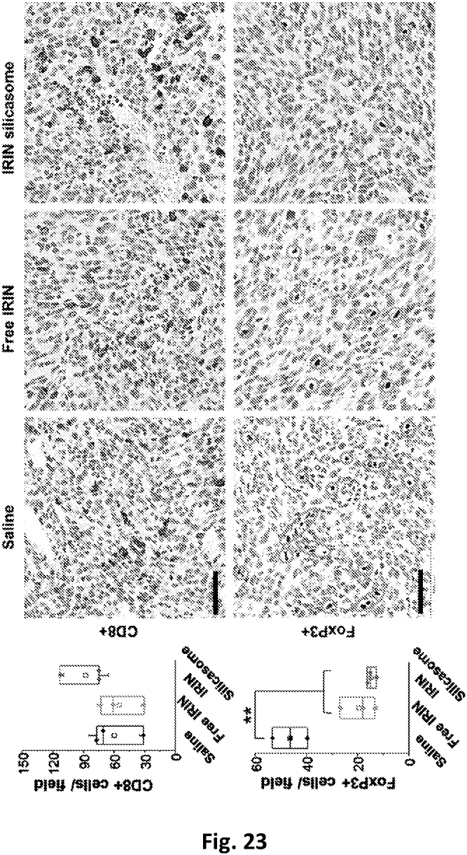

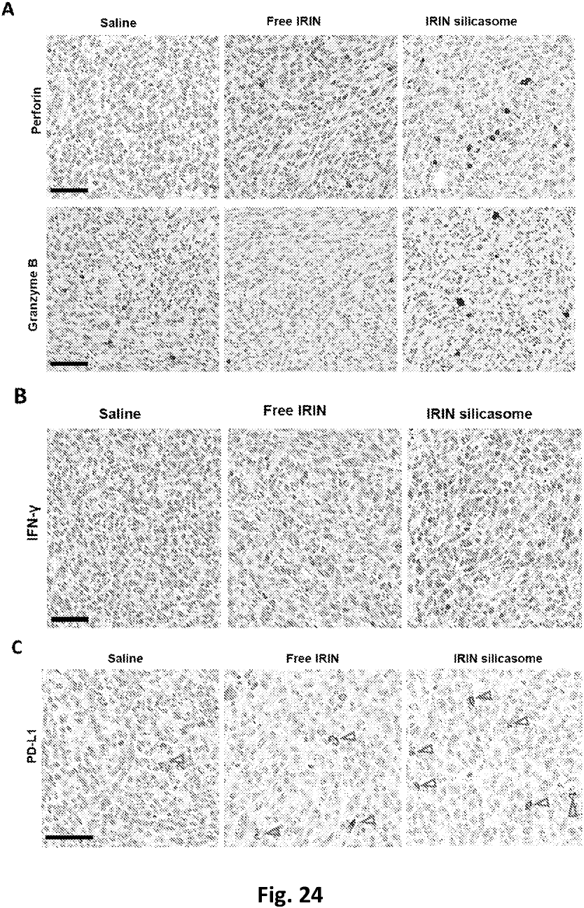

IHC analysis was also used to assess the expression of CD8 and FoxP3, as performed in the vaccination experiment (FIG. 2, panel D). While the silicasome treatment showed a marginal effect on the CD8⁺ cell number, there was a dramatic reduction of Treg numbers at the tumor site (FIG. 23). This resulted in a significant increase in the CD8⁺/Treg ratio (p < 0.001) compared to free drug or the saline control (FIG. 6, panel A). We also confirmed increased staining for perforin (p < 0.01) and granzyme B (p < 0.05) at the tumor site of animals treated with the IRIN silicasome compared to free drug or the saline control (FIG. 6, panels B and C). Representative IHC images appear in FIG. 24, panel A. Assessment of IFN-γ production in the TME showed a significant increase in response to treatment by free and encapsulated IRIN, the latter being shown to be significantly (p < 0.05) higher than free drug (FIG. 6, panel D). Representative IHC images appear in FIG. 24, panel B. Similar response profiles were obtained during the assessment of PD-L1 expression (FIG. 6, panel E and 24, panel C). This is congruent with the level of IFN-γ production, which is a robust inducer of PD-L1 expression^{[24]}.

All considered, the IRIN silicasome was more effective than free drug for the ability to induce innate and adaptive anti-PDAC immune responses at the tumor site. We have previously demonstrated that this is the result of improved pharmacokinetics and drug delivery by the silicasome as a result of its increased carrier stability, circulatory half-life, and ability to transcytose to the PDAC site^{[16]}.

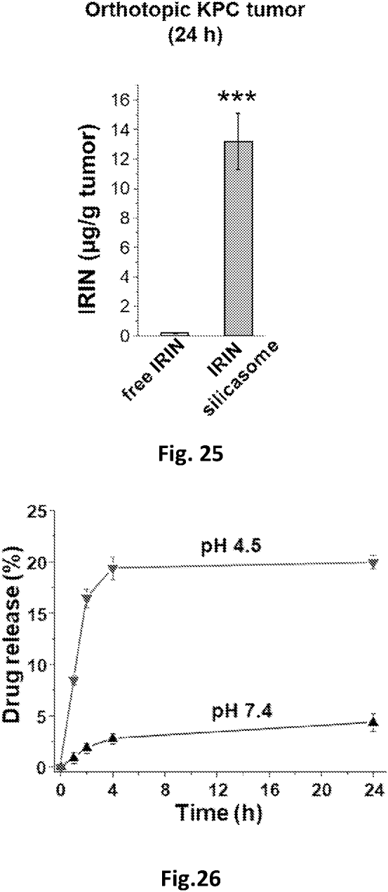

Utilizing LC-MS, we confirmed that the amount of delivered IRIN is at least a log-fold higher for encapsulated delivery compared to the free drug, as determined by IV injection of a drug equivalent of 40 mg/kg IRIN, followed by tumor harvesting 24 hours later (FIG. 25). This is consistent with our previous observations in the KPC model^{[16c]}. While it is difficult to quantify the amount of free drug in the tumor site, including in cancer cells and the surrounding interstitium, it is reasonable to expect that the acidifying conditions in the PDAC matrix^{[38]} will assist drug release, as demonstrated in our abiotic study showing that ~20% of the encapsulated irinotecan is released within 4 hrs at a pH of 4.5 (FIG. 26).

### Comparison of the Combination Immunotherapy Response of the IRIN Silicasome vs ONIVYDE®

We also performed a second survival in the KPC orthotopic model to compare the effect of encapsulated IRIN delivery by the silicasome vs. the ONIVYDE® liposome^{[3]} in the absence and presence of anti-PD-1 treatment. The study design, dosimetry considerations, and frequency of treatment administration were the same as in FIG. 4, panel B, with minor modifications (FIG. 7). While ONIVYDE® improved survival outcome compared to saline, the IRIN silicasome showed additional survival benefit over ONIVYDE®. Moreover, combination therapy with anti-PD-1 significantly extended the animal life span during treatment with the IRIN silicasome (comparable to FIG. 4, panel B), which was significantly better (p < 0.05) than the effect of anti-PD-1 co-administration with ONIVYDE®. The response of ONIVYDE® vs. ONIVYDE® plus anti-PD1antibody was non-significant (p = 0.22). All considered, these data demonstrate that IRIN silicasome plus anti-PD-1 combination therapy outperforms ONIVYDE® plus anti-PD-1.

### Discussion

In this study, we demonstrate that irinotecan is capable of triggering a chemo-immunotherapy response in an orthotopic KPC model. Not only is the response more robust during drug delivery by silicasome but considerably augmented in combination with an anti-PD-1 antibody. We also show that the immunogenic effects of IRIN, either in free for encapsulated form, can be ascribed to its effect on neutralizing the acidic pH of lysosomes. This early effect leads to autophagy inhibition and the triggering of an ER stress response that culminates in immunogenic cell death and PD-L1 expression. These immunogenic effects were confirmed by the ability of exposed KPC cells to trigger a vaccination response in vivo. The ability to induce an anti-tumor immune response was further confirmed in an orthotopic KPC tumor model, in which the delivery of IRIN to the tumor site by a silicasome could be seen to induce ICD markers, in addition to increasing the CD8/Treg ratio and PD-L1 expression. Moreover, response augmentation by anti-PD-1 antibodies was seen to significantly prolong animal survival, much better than the use of anti-PD-1 in combination with free drug or ONIVYDE®. These results suggest that it is feasible to improve PDAC survival with the IRIN silicasome through a chemo-immunotherapy effect that generates a “hot” tumor microenvironment that can be further exploited by immune checkpoint therapy.

IRIN has been used to treat solid tumors for approximately 30 years, particularly in patients with PDAC, colorectal and certain types of lung cancer^{[39]}. The classic mode of action (MOA) of this cytotoxic alkaloid is its conversion to SN-38, which functions as a topoisomerase I inhibitor, capable of inducing single and double strand DNA breaks ^{[40]}. It is not a surprise, therefore, that most studies addressing IRIN anticancer effects have focused on damage to the cell nucleus, while paying little attention to extranuclear effects ^{[39-40]} . However, studies of the impact of IRIN on colon cancer cell death have revealed evidence of “lysosomal leakage” and a possible impact on autophagy ^{[41]} . We now characterize this as an impact on autophagy flux due to the weak basic properties of IRIN. This leads to alkalization of the lysosomal pH by the free as well as the encapsulated drug, resulting in interference in organelle fusion with autophagosomes (FIGS. 1 and 3). Subsequent accumulation of p62, results in increased expression of PD-L1 (FIGS. 1 and 3). This distinguishes IRIN from most classic ICD inducers that are capable of inducing rather than interfering in autophagy ^{[12f, 14a]}. While the exact mechanism by which p62 induces PD-L1 expression requires further study, some evidence has been previously obtained that this could involve NF-κB activation^{[23]}, which is in keeping with our data showing increased phosphorylation of the p65 NF-κB subunit (FIG. 11). In addition to activating a potential immune escape mechanism, IRIN is also capable of inducing a type of immunogenic cell death linked to ER stress (FIG. 2). This is important since the drug until now has been depicted as having an ICD status that is “non-determined”^{[12e]}. We do show, however, that IRIN is capable of inducing CRT expression and HMGB1 release, in addition to the ability to generate a robust vaccination and life-prolonging immune response in the orthotopic PDAC model (FIG. 5, panels B-D). This equivalent to endogenous vaccination response that switches the poor immunogenic status of PDAC to tumor infiltration with activated cytotoxic T-cells, which are responsive to the use of anti-PD-1 (FIG. 4). All considered, this provides cumulative evidence that IRIN can indeed play an immunogenic role in cancer, as outlined in Table 61^{[42]}. Further evidence for the involvement of the immune system is the augmentation of MHC class I expression, concurrent with increased PD-L1 expression on mammary tumor cells ^{[42a]} . Moreover, in a mammary carcinoma model, it has also been demonstrated that IRIN can induce Treg depletion, in addition to the potential to synergize with anti-PD-L1^{[42a]}. Further, a subcutaneous MC38/gp100 colon cancer model was used to demonstrate that the antitumor efficacy of an IRIN-delivering liposome can be enhanced by ICI antibodies.^{[42b]}.

TABLE 6

Comparison of immunotherapy with encapsulated irinotecan carriers

| Ref | Treatment | Cancer Model | Anti-cancer effect | Key immunological and other biological findings of IRIN Increase In: |
|---|---|---|---|---|
| 1 | Free IRIN w/wo anti-PD-L1 | Subcutaneous FM3A Breast Cancer | Potency ranking: Free IRIN + a-PD-L1 > Free IRIN ≈ a-PD-L1 > saline | CD8/Treg ratio MHC class I PD-L1 |
| 2 | Liposomal IRIN (Onivyde/MM398) w/wo anti-PD-1/anti-PD-L1 | Subcutaneous MC38/gp100 Colon Cancer | Potency ranking: Liposomal IRIN + a-PD-1/ a-PD-L1 > liposomal IRIN a-PD-1/ a- | CD8/Treg ratio Granzyme B⁺ T cells |
| | | | PD-L1 alone ≈ saline | |
| 3 | Free IRIN w/wo anti-PD-1 | Orthotopic KPC pancreatic cancer | | CD8/Treg ratio PD-L1 expression Granzyme B+ T cells Perforin+ T cells IFN-γ production ICD (CRT, HMGB1) and ER stress Enrichment in acidic vesicles & autophagy inhibition |
| | ONIVYDE w/wo anti-PD-1 | | | |
| | IRIN silicasome w/wo anti -PD-1 | | | |

Refs: 1) Iwai et al. (2018) Oncotarget, 9: 31411; 2) McKenzie et al. (2018) J. Nat. Canc. Inst. 110, djx257; 3) This study.

Since its approval in 1998, the development of new IRIN formulations has been an area of great interest because of this drug’s high potency, which also leads to severe dose-limiting side effects^{[39]}. This includes severe neutropenia and gastro-intestinal toxicity, which limits IRIN use to PDAC patients with good clinical status. IRIN also has the shortcoming of large inter-individual PK variability that is accentuated by poor access to the desmoplastic PDAC tumor site^{[34]}. These challenges provided the basis for developing the liposomal formulation that received FDA approval as ONIVYDE®^{[3]}. While successful for inducing PDAC responses in the clinic, ONIVYDE® received a black box warning for residual drug toxicity, which could be due to drug leakage by the unsupported lipid bilayer^{[3]}. This observation is instrumental in the development of the IRIN-silicasome, which makes use of a supported lipid bilayer^{[16a, 16c]}. From this perspective, the silicasome can be viewed as a next-generation liposome (FIG. 3A), which is less leak, and capable of reducing bone marrow and gastro-intestinal toxicity compared to ONIVYDE®^{[3]} or an in-house liposome or ONIVYDE® in orthotopic KPC as well as colon cancer models, ^{[16a, 16c]}. Moreover, the increased stability of the silicasome also contributes to improved drug delivery at the desmoplastic PDAC tumor site^{[16a, 16c, 34c]}. In light of our demonstration of the immunogenic properties of IRIN, we introduce a novel treatment option for the use of the silicasome for PDAC treatment in the clinic in combination with ICI antibodies. We are also aware of an ongoing clinical trial that is looking at triple therapy (ONIVYDEO/5-fluorouracil/leucovorin) with anti-PD-1 antibody (pembrolizumab) plus a CXCR4 antagonist (BL-8040) for metastatic PDAC in patients who failed gemcitabine therapy (NCT02826486)^{[43]}. While the final report is not released yet, the preliminary data are encouraging, showing an objective response rate (ORR) of 32% for triple-therapy versus 17% for chemo combination only^{[3,} ^{43]}.

While most ICD responses to a chemo agent and the categorized as Type I responses, Type II responses have been linked to agents that generate a primary ER stress response and ROS production, e.g., hypericin-induced photodynamic therapy^{[44]} and Pt-N-heterocyclic carbene^{[45]}. We now demonstrate that IRIN, both as a free or an encapsulated drug format, induce robust ROS production and ER stress, and CHOP expression. Moreover, its lysosomal effects appear to precede the onset of nuclear damage, suggesting that the ER stress effect is a primary response. This particular constellation of findings would place IRIN category of Type II ICD inducer, which sets it apart from other chemotherapeutic agents. This could be advantageous from the perspective that Type II inducers are considered more robust inducers of anti-tumor immune responses^{[14b, 26]}. A more robust ICD effect holds clear advantages from the perspective that PDAC is generally considered a poorly immunogenic tumor^{[5b, 46]}. Not only does this open up the possibility for combination therapy with ICIs, as we show in the use of anti-PD-1 (FIGS. 4 and 7), but it also paves the way for considering the introduction of additional immunomodulators, including anti-CD40 antibodies, IDO-1 inhibitors, autophagic inhibitors, or small drug inhibitors of immune checkpoint pathways ^{[47]} . From this perspective, the creative use of the multifunctional silicasome platform promises to allow additional synergistic treatment combinations, for use in immunotherapy. Here it is important to consider the contribution of the dysplastic stroma, it’s high content of carcinoma-associated fibroblasts (CAFs) and myeloid derived dendritic cells to interference in the PDAC immune response^{[5, 10c, 46b]}. This can be accomplished by combinatorial therapy with TGF-β inhibitor (e.g. LY364947)^{[15b, 48]}, as well as CXCR4 antagonists (e.g. AMD 3100^{[49]} and BL-8040^{[43]}), which can disrupt adhesive tumor-stroma interactions and overcome T cell exclusion mechanism via targeting FAP-expressing CAFs, making PDAC more accessible to conventional drugs and cytotoxic T cells^{[43, 49-50].}

### Conclusions

In summary, we provide a novel explanation for the immunogenic effects of irinotecan. First, the weak basic drug neutralizes the acidic pH of the lysosome in KPC cells, leading to autophagy inhibition and upregulation of PD-L1 expression. A second linked effect is the delivery of a robust ER stress response which leads to cell death, characterized by ecto-CRT expression and the generation of immunological danger signals. Collectively, this culminates in an immunogenic cell death response accompanied by PD-L1 expression. The in vivo relevance is that this allowed us to induce an ICD response in an orthotopic KPC model by using encapsulated delivery of IRIN in a silicasome carrier. The response could be augmented by anti-PD-1 treatment, leading to a pronounced survival improvement compared to anti-PD-1 combination therapy with free drug or a liposome composition. Our discovery introduces a major additional avenue for PDAC chemotherapy.

### Methods

### Materials

Tetraethylorthosicate (TEOS), triethanolamine (TEA-ol), triethylamine (TEA) cetyltrimethylammonium chloride solution (CTAC, 25 wt% in water), Dowex 50WX8 resin, and chloroquine diphosphate salt were purchased from Sigma-Aldrich, USA. Sucrose octasulfate (SOS) sodium salt was purchased from Toronto Research Chemicals, Inc, Canada. 1,2-Distearoyl-sn-glycero-3-phosphocholine (DSPC), 1,2-distearoyl-sn-glycero-3-phospho-ethanol amine-N-[methoxy(polyethylene glycol)-2000] (ammonium salt) (DSPE-PEG₂₀₀₀), and cholesterol (Chol) were purchased from Avanti Polar Lipids, USA. Sepharose CL-4B was purchased from GE Healthcare, USA. Irinotecan hydrochloride trihydrate, oxaliplatin, doxorubicin hydrochloride salt, paclitaxel and rapamycin were purchased from LC Laboratories, USA. Tunicamycin was purchased from Cell Signaling Technology. ONIVYDE® (Ipsen Biopharmaceuticals, Inc., 4.3 mg/mL irinotecan free base, 10 mL/vial) was purchased through the UCLA health pharmacy. Murine anti-PD-1 antibody (#BE0146) and dilution buffer (#IP0070) in InVivoPure were purchased Bio X Cell. Penicillin, streptomycin, Dulbecco’s modified Eagle medium (DMEM) and LysoTracker® Red DND-99 (L7528), and Fluo-4 AM (F14201) were purchased from Invitrogen. Cellular ROS Assay Kit (Red) (ab186027) was purchased from Abcam. Fetal bovine serum (FBS) was purchased from Gemini Bio Products. Murine IFN-γ was purchased from R&D (Minneapolis, MN). Matrigel™ Matrix Basement Membrane was purchased from BD Bioscience.

### Cell Culture

The KPC pancreatic adenocarcinoma cell line, which was derived from a spontaneous tumor originating in a transgenic Kras^{LSL-G12D/+}; Trp53^{LSL-R172H/+}; Pdx-1-Cre mouse (B6/129 background)^{[16, 17b]}, was cultured in DMEM, containing 10% FBS, 100 U/mL penicillin, 100 µg/mL streptomycin, 2 mM L-glutamine and 1 mM sodium pyruvate. To allow bioluminescence tumor imaging, the cells were permanently transfected with a luciferase-based lentiviral vector in the UCLA vector core facility, followed by a limiting dilution cloning as we previously described. ^{[16a]} PANC-1 cells were obtained from the American Type Culture Collection (ATCC), and cultured under similar conditions as KPC cells.

### Use of Confocal Microscopy to Study the Intracellular Distribution and Alkalinizing Effect of IRIN-Treated KPC Cells

Approximately 1.5×10⁴ KPC cells were seeded in µ-Slide 8 well (ibidi, 80826). To reveal the intracellular distribution of IRIN, attached KPC cells were treated with IRIN (300 µM) for 24 h. Cell membranes were stained using an Alexa Fluor® 594 conjugated WGA dye (Invitrogen™, W11262) at 2 µg/mL for 10 minutes. After washing, the cells were visualized using a Leica SP8-MD confocal microscope under the 100× objective lens. The excitation and emission wavelengths for IRIN were 405 nm and 440~490 nm, respectively^{[51]}

To demonstrate the alkalinizing cellular effect of IRIN, we used LysoTracker® Red DND-99. IRIN-treated KPC cells were washed and replenished with fresh media containing 100 nM DND-99 dye and 5 µg/mL Hoechst 33342 for nuclear staining. The cells were incubated at 37° C. for 0.5 h, followed by washing with phenol red free media. The cells were then visualized using a Leica SP8-MD confocal microscope under the 100× objective lens.

### Use of IF Staining to Demonstrate the Effect of IRIN on Autophagy and PD-L1 Expression in KPC Cells

KPC cells were seeded in the µ-Slide 8 well. After cell attachment, the cells were treated by IRIN at 300 µM for 24 h. Control treatments included exposure to PBS, chloroquine (32 µM), rapamycin (100 nM) and IFN-γ (10 ng/ml). Before IF staining, the cells were washed with PBS and fixed with 4% paraformaldehyde at RT for 15 minutes. Cells were treated in 1% BSA (blocking reagent) plus 0.2% triton-X100 in PBS for 30 minutes. Subsequently, the cells were incubated with primary antibody that recognizes LC3B (Cell Signaling #2775, 1:200) or p62/SQSTM1 (Cell Signaling #23214, 1:800) in 1% BSA containing PBS solution at 4° C. overnight. The sample was washed twice in PBS and further stained using an Alexa Fluor® 488 conjugated goat anti-rabbit secondary antibody (Thermo Fisher, A-11008, 1:1000) and 5 µg/mL Hoechst 33342 dye for 1 h. Cell surface staining for PD-L1 assessment was carried out in fixed (4% paraformaldehyde) cells, using a staining process similar to the above protocol. Primary PD-L1 antibody (Abcam, ab213480, 1: 500) and Alexa Fluor® 594 conjugated goat anti-rabbit secondary antibody (Thermo Fisher, A-11012, 1:1000) were used. The cells were visualized using a Leica SP8-MD confocal microscope under the 100x objective lens.

### Western Blotting

To confirm the confocal staining results for LC3B, p62 and PD-L1, western blotting experiments were performed in KPC cells exposed to IRIN (300 µM) or OX (500 µM) for 24 hr. Another experiment also looked at the dose-dependent effect of IRIN at concentrations of 75, 150, 300 and 600 µM for 24 h. Briefly, ~2×10⁵ KPC cells per well were seeded in 6 well-plates. After drug exposure, KPC cells were harvested and treated with cold RIPA lysis buffer (Cell Signaling # 9806S), supplemented with a cocktail of protease and phosphatase inhibitors (Cell Signaling #5872) and incubated on ice for 30 mins. After centrifugation of the lysates at 12,000 rpm for 10 min, protein concentration was quantified by a Bradford assay (Biorad). Equal amounts of protein in the supernatants were loaded onto a 10-20% Tris-glycine SDS-PAGE gel (Invitrogen, Grand Island, NY). The proteins were subsequently transferred to a PVDF membrane. The membrane was blocked with 5% non-fat dry milk/TBST, before incubation with primary and HRP-conjugated secondary antibodies. The primary antibodies included: LC3B (Cell Signaling #2775), p62/SQSTM1 (Cell Signaling #5114), NF-κB p65 (Cell Signaling #8242), Phospho-NF-xB p65 (Cell Signaling #3033) and PD-L1 (Abcam, ab213480). The blots were developed by soaking in ECL substrate (Thermo Fisher Scientific). Densitometric analysis of each protein band on the film was quantified by ImageJ software and normalized to the intensity of a corresponding housekeeping protein.

Western blotting was also used to detect the ER stress marker, CHOP, and Cleaved Caspase-3 (CC-3). In this case, KPC cells were treated with IRIN at the indicated concentrations (75- 600 µM) for 24 h. In a separate experiment, KPC cells were treated with IRIN (300 µM) and tunicamycin (10 µM) for 4 h. Primary antibodies were purchased from Cell Signaling Technology: β-actin (#3700), vinculin (#13901), CHOP (#2895), and Cleaved Caspase-3 (#9664).

### Measurement of the IRIN Effect on CRT Expression and HMGB1 Release

7.5×10⁴ KPC cells were seeded into 24-well plates. After cell attachment, KPC cells were treated with IRIN (300 µM), OX (500 µM), DOX (20 µM or PTX (12 µM for 24 h. The cell culture media were collected in 1.5 mL tube and spun down (2,000 rpm for 5 min) to collect the supernatants for HMGB1 detection by an ELISA kit (Catalog# ST51011, IBL International GmbH). Surface CRT expression was measured by flow cytometry in the same experiment, as previously described. ^{[27]} Briefly, the loosely attached cells were combined with trypsin-treated adhered cells. The cells were washed in cold PBS and then stained with a primary anti-CRT antibody (Abcam, ab2907, 1: 140) in 200 µL BD staining buffer for 0.5 h on ice. The cells were washed in cold PBS and stained with an Alexa Fluor® 680-conjugated secondary antibody (LifeScience Technologies #A21244) for 30 min on ice. After washing in cold PBS, the cells were assessed in a LSRII flow cytometer (BD Biosciences).

### Animal Purchase and Study Approval

Female B6/129SF1/J mice (JAX 101043) were purchased from The Jackson Laboratory, and maintained under pathogen-free conditions. All animal experiments were performed according to protocols approved by the UCLA Animal Research Committee.

### Assessment of the Immunogenic Effects of IRIN in a Vaccination Experiment

The vaccination schedule is highlighted in FIG. 2, panel C. Eight million KPC cells were seeded in a tissue culture dish. After cellular attachment, IRIN (300 µM or OX (500 µM were added for 24 h. Cells were collected and washed before resuspended in 0.8 mL cold PBS. For vaccination, each mouse received subcutaneous (SC) injection of a 100 µL suspension of chemo-treated cells in the right flank. Control animals received SC injection with 0.1 mL PBS. The vaccination was repeated after 7 days. Fourteen days after the 1^{st} injection, the animals received SC injection of normal KPC cells (1 million cells in 0.1 mL PBS) in the contralateral (left) flank. Tumor growth was measured by a digital caliper every 2-3 days, and the tumor volume calculated according to the formula: length × width² /2. Animals were sacrificed on day 26 and the tumors were collected, weighed and fixed in 10% formalin, followed by paraffin embedding and sectioning to derive to 4 µm thick slices for IHC analysis. Primary antibodies to CD8 (#14-0808-82) and FoxP3 (#13-5773-82) were purchased from ThermoFisher. IHC staining was performed in the UCLA Translational Pathology Core Laboratory (TPCL). The slides were scanned and images assessed by using Aperio ImageScope software (Leica).

### Synthesis, Purification, and Characterization of IRIN Silicasomes

The irinotecan-loaded silicasomes were prepared as previously reported described^{[16c]} Briefly, bare MSNPs were synthesized at 18 L scale and purified by extensive acidic ethanol washing to remove the CTAC detergent^{[16c]}.The trapping agent (TEA₈SOS) was prepared from a sucrose octasulfate sodium salt. For lipid coating, 40 mg/mL of the purified MSNPs, exposed to 80 mM TEA₈SOS solution for soaking in, were added to an ~50% (w/v) lipid solution. This ethanol suspended solution contained DSPC/Chol/DSPE-PEG₂₀₀₀, in the molar ratio of 3:2:0.15. This yields a MSNP:lipid ratio of 1:1.25 (w/w). The suspension was introduced by a flow pump into a flow cell (Sonics & Materials, Inc., #53630-0651) that provides probe sonication (Ultrasonic Processor Model VCX500, 80% amplitude) at a 15 s/15 s on/off cycle and a flow rate of 5 mL/min^{[16c]}. In order to remove the free trapping agent, the sample was purified through centrifugation (4,000 rpm for 5 min), followed by purification, using size exclusion chromatography. For IRIN import, TEA₈SOS-loaded particles were mixed with IRIN and incubated at 65° C. for 1 hour. The IRIN silicasomes were purified and filtered across a 0.2 µm filter for sterilization.

The final product was fully characterized as previously described by us^{[16c]} Briefly, the loading capacity was calculated as the weight ratio of irinotecan to MSNP. MSNP mass was determined by TGA. Particle hydrodynamic size and zeta potential were measured by a ZETAPALS instrument (Brookhaven Instruments Corporation). The final product was visualized by cryoEM (TF20 FEI Tecnai-G2) to confirm the uniformity and integrity of the coated lipid bilayer. A chromogenic LAL assay (QCL-1000 300 Test Kit, Lonza) was performed to test the endotoxin levels. Sterilization of the final product was confirmed by microbial (HPC Count sampler, Millipore Corp., MHPC10025), yeast and mold counting (Yeast and mold sampler, Millipore Corp., MY0010025) tests.

### Assessment of Cellular Responses to the IRIN Silicasome

The of the IRIN silicasome on lysosomal alkalization was performed at drug concentrations of 75 µM and 300 µM in KPC cells. Empty silicasomes (in which a 500 µg/mL particle dose is representative of an encapsulated IRIN dose of 300 µM) was included as control. The assessment of LC3B, p62 and PD-L1 immunoblotting and IF staining were performed in KPC cells during treatment with the IRIN silicasome or empty silicasomes, as described for the free drug.

### Assessment of the Treatment Response to Combination Therapy With the IRIN Silicasome Plus Anti-PD-1 in an Orthotopic KPC Tumor Model

The KPC-derived orthotopic tumor model in immunocompetent B6129SF1/J mouse was established as described in FIG. 4, panel A. ^{[16]} Briefly, 30 µL of DMEM/Matrigel (1:1 v/v), containing ~1×10⁶ KPC-luc cells, was injected into the tail of the pancreas in female B6129SF1/J mice (8~10 weeks) by a short survival surgery procedure.^{[16]} In the first survival experiment (FIG. 4, panel B), tumor-bearing mice were randomly assigned into 6 groups (n = 5-7) and received IV injection of the IRIN formulations at API dose of 40 mg/kg. Anti-PD-1 antibody was injected at 100 µg/animal IP. To assess survival rate, animals were monitored daily up to the stage of spontaneous death or approaching moribund status (defined in FIG. 4, panel A)^{[16, 36]}. The survival data were plotted as Kaplan-Meier curves, followed by data analysis to derive mean survival time. Statistical analysis and p values were obtained by Log Rank testing (Mantel-Cox), using GraphPad Prism 7.00 software.

### Assessment of Immune Parameters in Response to Silicasome Treatment in the Orthotopic KPC Tumor Model

Tumor-bearing mice received IV injection to deliver an IRIN dose of 40 mg/kg (free IRIN or IRIN silicasome) per injection every 3 days for a total of 3 administrations. Control animals received saline only. Animals were sacrificed 72 h after the last injection. To confirm the impact on tumor growth, ex vivo bioluminescence imaging was performed to assess image intensity at the primary and metastatic tumor sites. Primary tumors were fixed in 10% formalin, followed by paraffin embedding and sectioning to provide 4 µm slices for IHC analysis in the UCLA Translational Pathology Core Laboratory (TPCL). The slides were scanned and images were assessed by using Aperio ImageScope software (Leica). Primary antibodies to CD8 (#14-0808-82) and FoxP3 (#13-5773-82) were purchased from ThermoFisher; CRT (ab2907), while antibodies to HMGB1 (ab18256), granzyme B (ab4059), perforin (ab16074) and IFN-γ (ab9657) were purchased from Abcam. The antibody to LC-3 (#0231-100/LC3-5F10) was purchased from Nanotools, while the antibody to PD-L1 (#64988) was purchased from Cell Signaling Technology.

### Assessment of Anti-PD1 Combination Therapy With the Silicasomes vs ONIVYDE® in the Orthotopic KPC Model

The treatment schedule and frequency of administration are outlined in FIG. 7. Orthotopic KPC tumor-bearing mice were randomly assigned into 6 groups (n = 6). The treatment groups included: animals receiving IV injection of IRIN (ONIVYDE® or IRIN silicasome) at 40 mg/kg; IP injections of anti-PD-1 antibody monotherapy (100 µg/injection); or an anti-PD-1/chemo combination, for a total of 6 administrations. Kaplan-Meier analysis was performed as described in FIG. 6

### Statistical Analysis

Comparative analysis of differences between groups was performed using the 2-tailed Student’s t-test (Excel software, Microsoft) for two-group comparison. One-way ANOVA followed by a Tukey’s test (Origin software, OriginLab) was performed for multiple group comparisons. Data were expressed as mean ± SD or SEM, as stated in the figure legends. The survival analysis was performed by Log Rank testing (Mantel-Cox), using GraphPad Prism 7.00 software. A statistically significant difference was considered at *p < 0.05.

### REFERENCES

a) F. Bray, J. Ferlay, I. Soerjomataram, R. L. Siegel, L. A. Torre, A. Jemal, CA Cancer J Clin 2018, 68, 394; b) R. L. Siegel, K. D. Miller, A. Jemal, CA Cancer J Clin 2019, 69, 7.
a) F. Yang, C. Jin, D. L. Fu, A. L. Warshaw, World J Gastroenterol 2019, 25, 2839; b) T. Conroy, F. Desseigne, M. Ychou, O. Bouche, R. Guimbaud, Y. Becouarn, A. Adenis, J. L. Raoul, S. Gourgou-Bourgade, C. de la Fouchardiere, J. Bennouna, J. B. Bachet, F. Khemissa-Akouz, D. Pere-Verge, C. Delbaldo, E. Assenat, B. Chauffert, P. Michel, C. Montoto-Grillot, M. Ducreux, U. Groupe Tumeurs Digestives of, P. Intergroup, N Engl J Med 2011, 364, 1817; c) M. Suker, B. R. Beumer, E. Sadot, L. Marthey, J. E. Faris, E. A. Mellon, B. F. El-Rayes, A. Wang-Gillam, J. Lacy, P. J. Hosein, S. Y. Moorcraft, T. Conroy, F. Hohla, P. Allen, J. Taieb, T. S. Hong, R. Shridhar, I. Chau, C. H. van Eijck, B. G. Koerkamp, The Lancet. Oncology 2016, 17, 801; d) T. Conroy, P. Hammel, M. Hebbar, M. Ben Abdelghani, A. C. Wei, J. L. Raoul, L. Chone, E. Francois, P. Artru, J. J. Biagi, T. Lecomte, E. Assenat, R. Faroux, M. Ychou, J. Volet, A. Sauvanet, G. Breysacher, F. Di Fiore, C. Cripps, P. Kavan, P. Texereau, K. Bouhier-Leporrier, F. Khemissa-Akouz, J. L. Legoux, B. Juzyna, S. Gourgou, C. J. O’Callaghan, C. Jouffroy-Zeller, P. Rat, D. Malka, F. Castan, J. B. Bachet, C. C. T. Grp, U.-G.-P. Grp, New England Journal of Medicine 2018, 379, 2395; e) Q. P. Janssen, S. Buettner, M. Suker, B. R. Beumer, P. Addeo, P. Bachellier, N. Bahary, T. Bekaii-Saab, M. A. Bali, M. G. Besselink, B. A. Boone, I. Chau, S. Clarke, M. Dillhoff, B. F. El-Rayes, J. M. Frakes, D. Grose, P. J. Hosein, N. B. Jamieson, A. A. Javed, K. Khan, K. P. Kim, S. C. Kim, S. S. Kim, A. H. Ko, J. Lacy, G. A. Margonis, M. D. McCarter, C. J. McKay, E. A. Mellon, S. Y. Moorcraft, K. I. Okada, A. Paniccia, P. J. Parikh, N. A. Peters, H. Rabl, J. Samra, C. Tinchon, G. van Tienhoven, E. van Veldhuisen, A. Wang-Gillam, M. J. Weiss, J. W. Wilmink, H. Yamaue, M. Y. V. Homs, C. H. J. van Eijck, M. H. G. Katz, B. Groot Koerkamp, Journal of the National Cancer Institute 2019, 111, 782
A. Wang-Gillam, C. P. Li, G. Bodoky, A. Dean, Y. S. Shan, G. Jameson, T. Macarulla, K. H. Lee, D. Cunningham, J. F. Blanc, R. A. Hubner, C. F. Chiu, G. Schwartsmann, J. T. Siveke, F. Braiteh, V. Moyo, B. Belanger, N. Dhindsa, E. Bayever, D. D. Von Hoff, L. T. Chen, N.-S. Group, Lancet 2016, 387, 545
a) S. L. Topalian, F. S. Hodi, J. R. Brahmer, S. N. Gettinger, D. C. Smith, D. F. McDermott, J. D. Powderly, R. D. Carvajal, J. A. Sosman, M. B. Atkins, New England Journal of Medicine 2012, 366, 2443; b) X. Wang, Z. Bao, X. Zhang, F. Li, T. Lai, C. Cao, Z. Chen, W. Li, H. Shen, S. Ying, Oncotarget 2017, 8, 59901; c) L. Chen, X. Han, The Journal of clinical investigation 2015, 125, 3384
a) D. Kabacaoglu, K. J. Ciecielski, D. A. Ruess, H. Algul, Front Immunol 2018, 9, 1878; b) A. Rosenberg, D. Mahalingam, Journal of gastrointestinal oncology 2018, 9, 143; c) R. J. Torphy, Y. Zhu, R. D. Schulick, Ann Gastroenterol Surg 2018, 2, 274
Z. I. Hu, J. Shia, Z. K. Stadler, A. M. Varghese, M. Capanu, E. Salo-Mullen, M. A. Lowery, L. A. Diaz, Jr., D. Mandelker, K. H. Yu, A. Zervoudakis, D. P. Kelsen, C. A. Iacobuzio-Donahue, D. S. Klimstra, L. B. Saltz, I. H. Sahin, E. M. O’Reilly, Clin Cancer Res 2018, 24, 1326
a) K. C. Soares, A. A. Rucki, A. A. Wu, K. Olino, Q. Xiao, Y. Chai, A. Wamwea, E. Bigelow, E. Lutz, L. D. Liu, S. Yao, R. A. Anders, D. Laheru, C. L. Wolfgang, B. H. Edil, R. D. Schulick, E. M. Jaffee, L. Zheng, J Immunother 2015, 38, 1; b) M. Tsukamoto, K. Imai, T. Ishimoto, Y. Komohara, Y. i. Yamashita, S. Nakagawa, N. Umezaki, T. Yamao, Y. Kitano, T. Miyata, Cancer science 2019, 110, 310; c) X. L. Liang, J. Sun, H. W. Wu, Y. F. Luo, L. L. Wang, J. L. Lu, Z. W. Zhang, J. C. Guo, Z. Y. Liang, T. H. Liu, Diagnostic Pathology 2018, 13, 5
a) C. Lu, A. V. Paschall, H. Shi, N. Savage, J. L. Waller, M. E. Sabbatini, N. H. Oberlies, C. Pearce, K. Liu, Journal of the National Cancer Institute 2017, 109; b) L. Wang, Q. Ma, X. Chen, K. Guo, J. Li, M. Zhang, World J Surg 2010, 34, 1059
L. Zheng, Vol. 109, JNCI: Journal of the National Cancer Institute, 2017, djw304
a) A. Henriksen, A. Dyhl-Polk, I. Chen, D. Nielsen, Cancer Treat Rev 2019, 78, 17; b) J. P. Neoptolemos, J. Kleeff, P. Michl, E. Costello, W. Greenhalf, D. H. Palmer, Nature reviews. Gastroenterology & hepatology 2018, 15, 333; c) R. A. Skelton, A. Javed, L. Zheng, J. He, J Surg Oncol 2017, 116, 55
a) K. M. Heinhuis, W. Ros, M. Kok, N. Steeghs, J. H. Beijnen, J. H. M. Schellens, Annals of oncology : official journal of the European Society for Medical Oncology 2019, 30, 219; b) M. Aglietta, C. Barone, M. B. Sawyer, M. J. Moore, W. H. Miller, Jr., C. Bagala, F. Colombi, C. Cagnazzo, L. Gioeni, E. Wang, B. Huang, K. D. Fly, F. Leone, Annals of oncology : official journal of the European Society for Medical Oncology 2014, 25, 1750; c) C. K. Looi, F. F. Chung, C. O. Leong, S. F. Wong, R. Rosli, C. W. Mai, Journal of experimental & clinical cancer research : CR 2019, 38, 162
a) A. D. Garg, D. Nowis, J. Golab, P. Vandenabeele, D. V. Krysko, P. Agostinis, Biochimica et biophysica acta 2010, 1805, 53; b) X. Zhao, K. Yang, R. Zhao, T. Ji, X. Wang, X. Yang, Y. Zhang, K. Cheng, S. Liu, J. Hao, H. Ren, K. W. Leong, G. Nie, Biomaterials 2016, 102, 187; c) J. Pol, E. Vacchelli, F. Aranda, F. Castoldi, A. Eggermont, I. Cremer, C. Sautes-Fridman, J. Fucikova, J. Galon, R. Spisek, E. Tartour, L. Zitvogel, G. Kroemer, L. Galluzzi, Oncoimmunology 2015, 4, e1008866; d) L. Galluzzi, A. Buque, O. Kepp, L. Zitvogel, G. Kroemer, Cancer Cell 2015, 28, 690; e) L. Bezu, L. C. Gomes-da-Silva, H. Dewitte, K. Breckpot, J. Fucikova, R. Spisek, L. Galluzzi, O. Kepp, G. Kroemer, Frontiers in immunology 2015, 6, 187; f) G. Kroemer, L. Galluzzi, O. Kepp, L. Zitvogel, Annu Rev Immunol 2013, 31, 51; g) C. Pfirschke, C. Engblom, S. Rickelt, V. Cortez-Retamozo, C. Garris, F. Pucci, T. Yamazaki, V. Poirier-Colame, A. Newton, Y. Redouane, Y. J. Lin, G. Wojtkiewicz, Y. Iwamoto, M. Mino-Kenudson, T. G. Huynh, R. O. Hynes, G. J. Freeman, G. Kroemer, L. Zitvogel, R. Weissleder, M. J. Pittet, Immunity 2016, 44, 343
O. Kepp, L. Menger, E. Vacchelli, C. Locher, S. Adjemian, T. Yamazaki, I. Martins, A. Q. Sukkurwala, M. Michaud, L. Senovilla, L. Galluzzi, G. Kroemer, L. Zitvogel, Cytokine & Growth Factor Reviews 2013, 24, 311
a) M. Michaud, I. Martins, A. Q. Sukkurwala, S. Adjemian, Y. Ma, P. Pellegatti, S. Shen, O. Kepp, M. Scoazec, G. Mignot, S. Rello-Varona, M. Tailler, L. Menger, E. Vacchelli, L. Galluzzi, F. Ghiringhelli, F. di Virgilio, L. Zitvogel, G. Kroemer, Science 2011, 334, 1573; b) D. V. Krysko, A. D. Garg, A. Kaczmarek, O. Krysko, P. Agostinis, P. Vandenabeele, Nature reviews. Cancer 2012, 12, 860
a) H. Han, D. Valdeperez, Q. Jin, B. Yang, Z. Li, Y. Wu, B. Pelaz, W. J. Parak, J. Ji, ACS Nano 2017, 11, 1281; b) H. Meng, Y. Zhao, J. Dong, M. Xue, Y. S. Lin, Z. Ji, W. X. Mai, H. Zhang, C. H. Chang, C. J. Brinker, J. I. Zink, A. E. Nel, ACS Nano 2013, 7, 10048; c) J. Nam, S. Son, K. S. Park, W. P. Zou, L. D. Shea, J. J. Moon, Nature Reviews Materials 2019, 4, 398; d) J. Shi, P. W. Kantoff, R. Wooster, O. C. Farokhzad, Nature reviews. Cancer 2017, 17, 20; e) E. L. Siegler, Y. J. Kim, P. Wang, Journal of Cellular Immunotherapy 2016, 2, 69; f) W. Jiang, C. A. von Roemeling, Y. X. Chen, Y. Q. Qie, X. J. Liu, J. Z. Chen, B. Y. S. Kim, Nature Biomedical Engineering 2017, 1, 1; g) S. Musetti, L. Huang, ACS Nano 2018, 12, 11740; h) Q. Zhang, F. Liu, K. T. Nguyen, X. Ma, X. Wang, B. Xing, Y. Zhao, Advanced Functional Materials 2012, 22, 5144; i) G. Yang, S. Z. F. Phua, W. Q. Lim, R. Zhang, L. Feng, G. Liu, H. Wu, A. K. Bindra, D. Jana, Z. Liu, Advanced Materials 2019, 31, 1901513
a) X. S. Liu, A. Situ, Y. A. Kang, K. R. Villabroza, Y. P. Liao, C. H. Chang, T. Donahue, A. E. Nel, H. Meng, Acs Nano 2016, 10, 2702; b) X. Liu, P. Lin, I. Perrett, J. Lin, Y. P. Liao, C. H. Chang, J. Jiang, N. Wu, T. Donahue, Z. Wainberg, A. E. Nel, H. Meng, J Clin Invest 2017, 127, 2007; c) X. Liu, J. Jiang, R. Chan, Y. Ji, J. Lu, Y. P. Liao, M. Okene, J. Lin, P. Lin, C. H. Chang, X. Wang, I. Tang, E. Zheng, W. Qiu, Z. A. Wainberg, A. E. Nel, H. Meng, ACS Nano 2019, 13, 38
a) S. R. Hingorani, L. Wang, A. S. Multani, C. Combs, T. B. Deramaudt, R. H. Hruban, A. K. Rustgi, S. Chang, D. A. Tuveson, Cancer Cell 2005, 7, 469; b) W. W. Tseng, D. Winer, J. A. Kenkel, O. Choi, A. H. Shain, J. R. Pollack, R. French, A. M. Lowy, E. G. Engleman, Clin Cancer Res 2010, 16, 3684
H. Y. Wei, J. Song, H. Li, Y. Li, S. S. Zhu, X. D. Zhou, X. W. Zhang, L. Yang, Asian Journal of Pharmaceutical Sciences 2013, 8, 303
G. Griffiths, B. Hoflack, K. Simons, I. Mellman, S. Kornfeld, Cell 1988, 52, 329
a) A. Mahli, M. Saugspier, A. Koch, J. Sommer, P. Dietrich, S. Lee, R. Thasler, J. Schulze-Luehrmann, A. Luehrmann, W. E. Thasler, M. Muller, A. Bosserhoff, C. Hellerbrand, Gut 2018, 67, 746; b) A. Kawai, H. Uchiyama, S. Takano, N. Nakamura, S. Ohkuma, Autophagy 2007, 3, 154
a) G. Kroemer, M. Jaattela, Nature reviews. Cancer 2005, 5, 886; b) J. P. Luzio, P. R. Pryor, N. A. Bright, Nature reviews. Molecular cell biology 2007, 8, 622
C. Mauvezin, P. Nagy, G. Juhász, T. P. Neufeld, Nature communications 2015, 6, 1
X. Wang, W. K. K. Wu, J. Gao, Z. Li, B. Dong, X. Lin, Y. Li, Y. Li, J. Gong, C. Qi, Z. Peng, J. Yu, L. Shen, Journal of experimental & clinical cancer research : CR 2019, 38, 140
A. Garcia-Diaz, D. S. Shin, B. H. Moreno, J. Saco, H. Escuin-Ordinas, G. A. Rodriguez, J. M. Zaretsky, L. Sun, W. Hugo, X. Wang, G. Parisi, C. P. Saus, D. Y. Torrejon, T. G. Graeber, B. Comin-Anduix, S. Hu-Lieskovan, R. Damoiseaux, R. S. Lo, A. Ribas, Cell Rep 2017, 19, 1189
a) A. D. Garg, A. M. Dudek, G. B. Ferreira, T. Verfaillie, P. Vandenabeele, D. V. Krysko, C. Mathieu, P. Agostinis, Autophagy 2013, 9, 1292; b) A. D. Garg, P. Agostinis, Photochemical & photobiological sciences : Official journal of the European Photochemistry Association and the European Society for Photobiology 2014, 13, 474
a) A. D. Garg, A. M. Dudek-Peric, E. Romano, P. Agostinis, International Journal of Developmental Biology 2015, 59, 131; b) F. Radogna, M. Diederich, Biochem Pharmacol 2018, 153, 12
M. Obeid, A. Tesniere, F. Ghiringhelli, G. M. Fimia, L. Apetoh, J. L. Perfettini, M. Castedo, G. Mignot, T. Panaretakis, N. Casares, D. Metivier, N. Larochette, P. van Endert, F. Ciccosanti, M. Piacentini, L. Zitvogel, G. Kroemer, Nat Med 2007, 13, 54
a) C. M. Oslowski, F. Urano, in Methods in enzymology, Vol. 490, Elsevier 2011, p. 71; b) S. Oyadomari, M. Mori, Cell Death & Differentiation 2004, 11, 381; c) S. A. Oakes, F. R. Papa, Annual review of pathology 2015, 10, 173; d) M. M. Khan, W. L. Yang, P. Wang, Shock 2015, 44, 294
H. Yang, H. Shen, J. Li, L. W. Guo, Autophagy 2019, 15, 1539
E. Bahar, H. Kim, H. Yoon, International Journal of Molecular Sciences 2016, 17, 1558
S. Dolai, S. Pal, R. K. Yadav, S. Adak, Journal of Biological Chemistry 2011, 286, 13638
M. Cirone, M. S. Gilardini Montani, M. Granato, A. Garufi, A. Faggioni, G. D′Orazi, Journal of Experimental & Clinical Cancer Research 2019, 38, 262
O. Kepp, L. Senovilla, I. Vitale, E. Vacchelli, S. Adjemian, P. Agostinis, L. Apetoh, F. Aranda, V. Barnaba, N. Bloy, Oncoimmunology 2014, 3, e955691
a) H. Meng, A. E. Nel, Advanced drug delivery reviews 2018, 130, 50; b) A. Dimou, K. N. Syrigos, M. W. Saif, Ther Adv Med Oncol 2012, 4, 271; c) X. Liu, J. Jiang, H. Meng, Theranostics 2019, 9, 8018
D. C. Drummond, C. O. Noble, Z. X. Guo, K. Hong, J. W. Park, D. B. Kirpotin, Cancer Research 2006, 66, 3271
K. P. Olive, M. A. Jacobetz, C. J. Davidson, A. Gopinathan, D. McIntyre, D. Honess, B. Madhu, M. A. Goldgraben, M. E. Caldwell, D. Allard, K. K. Frese, G. Denicola, C. Feig, C. Combs, S. P. Winter, H. Ireland-Zecchini, S. Reichelt, W. J. Howat, A. Chang, M. Dhara, L. Wang, F. Ruckert, R. Grutzmann, C. Pilarsky, K. Izeradjene, S. R. Hingorani, P. Huang, S. E. Davies, W. Plunkett, M. Egorin, R. H. Hruban, N. Whitebread, K. McGovern, J. Adams, C. Iacobuzio-Donahue, J. Griffiths, D. A. Tuveson, Science 2009, 324, 1457
P. Tardi, S. Johnstone, N. Harasym, S. Xie, T. Harasym, N. Zisman, P. Harvie, D. Bermudes, L. Mayer, Leuk Res 2009, 33, 129
J. W. Wojtkowiak, J. M. Rothberg, V. Kumar, K. J. Schramm, E. Haller, J. B. Proemsey, M. C. Lloyd, B. F. Sloane, R. J. Gillies, Cancer Res 2012, 72, 3938
a) M. Femke, A. K. Goey, R. H. van Schaik, R. H. Mathijssen, S. Bins, Clinical pharmacokinetics 2018, 57, 1229; b) C. Bailly, Pharmacol Res 2019, 148, 104398
R. H. Mathijssen, R. J. van Alphen, J. Verweij, W. J. Loos, K. Nooter, G. Stoter, A. Sparreboom, Clin Cancer Res 2001, 7, 2182
S. John, J. Mls, M. Cervinka, E. Rudolf, Anti-Cancer Agent Me 2013, 13, 811
a) T. Iwai, M. Sugimoto, D. Wakita, K. Yorozu, M. Kurasawa, K. Yamamoto, Oncotarget 2018, 9, 31411; b) J. A. McKenzie, R. M. Mbofung, S. Malu, M. Zhang, E. Ashkin, S. Devi, L. Williams, T. Tieu, W. Peng, S. Pradeep, C. Xu, S. Zorro Manrique, C. Liu, L. Huang, Y. Chen, M. A. Forget, C. Haymaker, C. Bernatchez, N. Satani, F. Muller, J. Roszik, A. Kalra, T. Heffernan, A. Sood, J. Hu, R. Amaria, R. E. Davis, P. Hwu, Journal of the National Cancer Institute 2018, 110, 777
B. Bockorny, V. Semenisty, T. Macarulla, E. Borazanci, B. M. Wolpin, S. M. Stemmer, T. Golan, R. Geva, M. J. Borad, K. S. Pedersen, J. O. Park, R. A. Ramirez, D. G. Abad, J. Feliu, A. Munoz, M. Ponz-Sarvise, A. Peled, T. M. Lustig, O. Bohana-Kashtan, S. M. Shaw, E. Sorani, M. Chaney, S. Kadosh, A. Vainstein Haras, D. D. Von Hoff, M. Hidalgo, Nat Med 2020, 26, 878
a) T. Verfaillie, N. Rubio, A. D. Garg, G. Bultynck, R. Rizzuto, J. P. Decuypere, J. Piette, C. Linehan, S. Gupta, A. Samali, P. Agostinis, Cell Death Differ 2012, 19, 1880; b) A. D. Garg, D. V. Krysko, T. Verfaillie, A. Kaczmarek, G. B. Ferreira, T. Marysael, N. Rubio, M. Firczuk, C. Mathieu, A. J. Roebroek, W. Annaert, J. Golab, P. de Witte, P. Vandenabeele, P. Agostinis, EMBO J 2012, 31, 1062
D. Y. Wong, W. W. Ong, W. H. Ang, Angewandte Chemie 2015, 54, 6483
a) E. R. Lutz, A. A. Wu, E. Bigelow, R. Sharma, G. Mo, K. Soares, S. Solt, A. Dorman, A. Wamwea, A. Yager, Cancer immunology research 2014, 2, 616; b) M. Orth, P. Metzger, S. Gerum, J. Mayerle, G. Schneider, C. Belka, M. Schnurr, K. Lauber, Radiat Oncol 2019, 14, 141
a) S. Yasmin-Karim, P. T. Bruck, M. Moreau, S. Kunjachan, G. Z. Chen, R. Kumar, S. Grabow, S. K. Dougan, W. Ngwa, Front Immunol 2018, 9, 2030; b) I. Garrido-Laguna, M. Hidalgo, Nat Rev Clin Oncol 2015, 12, 319
M. R. Kano, Y. Bae, C. Iwata, Y. Morishita, M. Yashiro, M. Oka, T. Fujii, A. Komuro, K. Kiyono, M. Kaminishi, Proc Natl Acad Sci U S A 2007, 104, 3460
C. Feig, J. O. Jones, M. Kraman, R. J. Wells, A. Deonarine, D. S. Chan, C. M. Connell, E. W. Roberts, Q. Zhao, O. L. Caballero, S. A. Teichmann, T. Janowitz, D. I. Jodrell, D. A. Tuveson, D. T. Fearon, Proc Natl Acad Sci U S A 2013, 110, 20212
J. A. Burger, A. Peled, Leukemia 2009, 23, 43
a) S. T. Wang, H. P. Deng, P. Huang, P. Sun, X. H. Huang, Y. Su, X. Y. Zhu, J. Shen, D. Y. Yan, Rsc Advances 2016, 6, 12472; b) M. R. di Nunzio, Y. Douhal, J. A. Organero, A. Douhal, Physical chemistry chemical physics : PCCP 2018, 20, 14182.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating a cancer in a mammal, said method comprising:
administering to said mammal, or causing to be administered to said mammal, an effective amount of:
i) one or more checkpoint inhibitor(s); and
ii) one or more camptothecin analogs, and, optionally, or one or more autophagy inhibitors wherein said camptothecin analog, and one or more autophagy inhibitors, when present, are provided inside a delivery vehicle where said delivery vehicle comprises:
a nanoparticle comprising one or more cavities disposed within said nanoparticle and an outside surface where said one or more cavities are in fluid communication the outside surface of said nanoparticle;
said one or more camptothecin analogs, and said one or more autophagy inhibitors, when present, are disposed within said one or more cavities; and
a lipid bilayer is disposed on the surface of said nanoparticle where said lipid bilayer fully encapsulates the nanoparticle.

2. The method of claim 1, wherein said one or more checkpoint inhibitors and said one or more camptothecin analogs are administered to said subject simultaneously.

3. The method of claim 2, wherein said one or more checkpoint inhibitors and said one or more camptothecin analogs are administered in a combined formulation.

4. The method of claim 1, wherein said one or more checkpoint inhibitors and said one or more camptothecin analogs are administered at different times.

5. The method according to any one of claims 1-4, wherein said camptothecin analog comprises irinotecan.

6. The method according to any one of claims 1-5, wherein said camptothecin analog comprises a camptothecin analog other than irinotecan.

7. The method of claim 6, wherein said camptothecin analog comprises an analog selected from the group consisting of belotecan (CKD-602), topotecan, silatecan (db-67, ar-67), cositecan (bnp-1350), exatecan, lurtotecan, gimatecan (st1481), rubitecan, homocamptothecin, trastuzumab deruxtecan, Rubitecan, Beltecan, Exatecan, Lurtotecan, Gimatecan, Diflomotecan, Karenitecan, Silatecan, Namitecan, ZBH-1205, Elomotecan, DRF-1042, Delimotecan, NSC606985, Chimmitecan, Genz-644282, and non-CPT1.

8. The method according to any one of claims 6-7, wherein said camptothecin analog comprises a weakly basic analog.

9. The method according to any one of claims 6-7, wherein said camptothecin analog comprises a water soluble analog.

10. The method of claim 9, wherein said water soluble analog has a solubility of greater than 5 mg/mL in water, or greater than 8 mg/mL in water, or greater than 10 mg/mL in water, or greater than about 12 mg/mL in water or greater than about 15 mg/mL in water, or greater than about 20 mg/mL in water, or greater than about 22 mg/mL in water.

11. The method according to any one of claims 7-10, wherein said camptothecin analog comprises belotecan (CKD-602).

12. The method according to any one of claims 1-11, wherein said checkpoint inhibitor comprises one or more checkpoint inhibitors selected from the group consisting of a PD-L1 inhibitor, a PD-1 inhibitor, and a CTLA-4 inhibitor.

13. The method of claim 12, wherein said checkpoint inhibitor comprises one or more PD-L1 inhibitors.

14. The method of claim 13, wherein said checkpoint inhibitor comprises an anti-PD-L1 antibody.

15. The method of claim 14, wherein said checkpoint inhibitor comprises an anti-PD-L1 antibody selected from the group consisting of Atezolizumab, Avelumab, Durvalumab, BMS-936559, RG-7446. MPDL3280A, MEDI-4736, and MSB0010718C.

16. The method of claim 13, wherein said checkpoint inhibitor comprises a peptidic PD-L1 inhibitor.

17. The method of claim 16, wherein said PD-L1 inhibitor comprise a moiety selected from the group consisting of AUNP12, CA-170, and BMS-986189.

18. The method according to any one of claims 12-17, wherein said checkpoint inhibitor comprises a PD1 inhibitor.

19. The method of claim 18, wherein said checkpoint inhibitor comprises an anti-PD1 antibody.

20. The method of claim 19, wherein said checkpoint inhibitor comprises an anti-PD1 antibody selected from the group consisting of Nivolumab, Pembrolizumab, Cemiplimab, avelumab, durvalumab, and atezolizumab.

21. The method of claim 18, wherein said checkpoint inhibitor comprises an fc fusion with PD-L2.

22. The method of claim 21, wherein said checkpoint inhibitor comprises AMP224.

23. The method according to any one of claims 12-22, wherein said checkpoint inhibitor comprises CTLA-4 inhibitor.

24. The method of claim 23, wherein said CTLA-4 inhibitor comprises Ipilimumab.

25. The method according to any one of claims 1-11, wherein said checkpoint inhibitor comprises a bispecific antibody that binds to two checkpoint inhibitors, or an antibody that binds to a checkpoint inhibitor attached to a cytokine.

26. The method of claim 25, wherein said checkpoint inhibitor comprises a bispecific antibody that binds to two checkpoint inhibitors.

27. The method of claim 26, wherein said bispecific antibody comprises an antibody that binds to PD-1 attached to an antibody that binds to PD-L1, or an antibody that binds to PD-1 attached to an antibody that binds to CTLA4, or an antibody that binds to PD-L1 attached to an antibody that binds to CTLA4.

28. The method of claim 27, wherein said bispecific antibody comprises an antibody that binds to PD-1 attached to an antibody that binds to CTLA4.

29. The method of claim 25, wherein said checkpoint inhibitor comprises a cytokine attached to an antibody that binds to a checkpoint inhibitor.

30. The method of claim 29, wherein said checkpoint inhibitor comprises a cytokine attached to an antibody selected from the group consisting of anti-PD-1, anti-PD-L1, and CTLA4.

31. The method of claim 30, wherein said checkpoint inhibitor comprises cytokine attached to an anti-PD-1 antibody.

32. The method of claim 31, wherein said checkpoint inhibitor comprises an IL-7 attached to an anti-PD-1 antibody.

33. The method according to any one of claims 1-32, wherein said drug delivery vehicle contains one or more autophagy inhibitors.

34. The method of claim 33, wherein said one or more autophagy inhibitors comprises an agent selected from the group consisting of chloroquine, hydroxychloroquine, and a member of the bafilomycin family.

35. The method of claim 34, wherein said one or more autophagy inhibitors comprises chloroquine.

36. The method according to any one of claims 34-35, wherein said one or more autophagy inhibitors comprises hydroxychloroquine.

37. The method according to any one of claims 34-36, wherein said one or more autophagy inhibitors comprises a member of the bafilomycin family.

38. The method of claim 37 wherein said one or more autophagy inhibitors comprises a member of the bafilomycin family selected from the group consisting of bafilomycin A1, bafilomycin B1, bafilomycin B2, bafilomycin C1, bafilomycin C2, bafilomycin C1 amide, bafilomycin C2 amide, 9-hydroxybafilomycin D, 29-hydroxybafilomycin D, bafilomycin D, and bafilomycin E.

39. The method of claim 33, wherein said one or more autophagy inhibitors comprises one or more autophagy inhibitors shown in Table .

40. The method according to any one of claims 33-39, wherein said one or more autophagy inhibitors comprises said autophagy inhibitor comprises an autophagy-inhibiting nanoparticle.

41. The method of claim 40, wherein said autophagy-inhibiting nanoparticle comprises a metal or metal oxide, a rare earth or rare earth oxide, or silica.

42. The method of claim 41, wherein said autophagy-inhibiting nanoparticle comprises a metal or metal oxide.

43. The method of claim 41, wherein said autophagy-inhibiting nanoparticle comprises a metal.

44. The method of claim 43, wherein said autophagy-inhibiting nanoparticle comprise a metal selected from the group consisting of gold, silver, iron, copper, and titanium.

45. The method of claim 41, wherein said autophagy-inhibiting nanoparticle comprises a metal oxide.

46. The method of claim 45, wherein said autophagy-inhibiting nanoparticle comprises a metal oxide selected from the group consisting of zinc oxide, iron oxide, iron oxide/gold, copper oxide, titanium dioxide, and ferroferic oxide.

47. The method of claim 41, wherein said autophagy-inhibiting nanoparticle comprises a rare earth or rare earth oxide.

48. The method of claim 47, wherein said autophagy-inhibiting nanoparticle comprise a rare earth selected from the group consisting of scandium, yttrium, lanthanum, cerium, praseodymium, neodymium, promethium, samarium, europium, gadolinium, terbium, dysprosium, holmium, erbium, thulium, ytterbium, and lutetium.

49. The method of claim 47, wherein said autophagy-inhibiting nanoparticle comprise a rare earth oxide selected from the group consisting of cerium oxide, and europium hydroxide.

50. The method according to any one of claims 1-49, wherein said nanoparticle comprise a single cavity.

51. The method of claim 50, wherein said nanoparticle comprises a nanobowl.

52. The method according to any one of claims 1-49, wherein said nanoparticle comprises a plurality of cavities.

53. The method of claim 52, wherein said nanoparticle comprises a porous inorganic nanoparticle, a metal-organic framework nanoparticle, or a porous organic nanoparticle.

54. The method of claim 53, wherein said nanoparticle comprise a porous inorganic nanoparticle.

55. The method of claim 54, wherein said nanoparticle comprise a porous silica nanoparticle, a porous calcium carbonate nanoparticle, or a porous calcium phosphate nanoparticle.

56. The method of claim 55, wherein said nanoparticle comprises a porous silica nanoparticle.

57. The method of claim 56, wherein said nanoparticle comprises a mesoporous silica nanoparticle (MSN), a mesoporous organosilica nanoparticle (MONs), or a periodic mesoporous organosilica (PMO) nanoparticle.

58. The method of claim 57, wherein said nanoparticle comprises a mesoporous silica nanoparticle (MSN).

59. The method of claim 58, wherein said nanoparticle comprises undoped and unfunctionalized silica.

60. The method according to any one of claims 57-58, wherein said nanoparticle comprises a mesoporous silica /hydroxyapatite (MSNs/HAP) hybrid nanoparticle.

61. The method according to any one of claims 57-58, wherein said nanoparticle comprises a cleavable silsesquioxane, or a bridged silsesquioxane (BS).

62. The method according to any one of claims 57-58, wherein said nanoparticle comprises an inorganically doped silica.

63. The method of claim 62, wherein said nanoparticle comprises a calcium-, iron-, manganese-, or zirconium-doped silica.

64. The method according to any one of claims 57-58, wherein said nanoparticle comprises an imine-doped silica.

65. The method of claim 55, wherein said nanoparticle comprises a mesoporous calcium carbonate nanoparticle.

66. The method of claim 55, wherein said nanoparticle comprises a mesoporous calcium phosphate nanoparticle.

67. The method of claim 53, wherein said nanoparticle comprises a porous biocompatible polymer.

68. The method of claim 67, wherein said nanoparticle comprise a porous biocompatible polymer selected from the group consisting of polymers of the polyaryletherketone (PAEK) family (e.g., polyether ether ketone (PEEK), carbon reinforced PEEK, polyether ketone ketone (PEKK), PEKEKK (polyetherketoneetherketoneketone), polyaryletherketone (PAEK), polyetherketone (PEK), Polyetherketone Etherketone Ketone (PEKEKK), and the like), polycaprolactone (PCL), polylactic acid (PLA), polyglycolic acid (PGA), polyphenylene, self-reinforced polyphenylene, polyphenylsulphone, polysulphone, polyethylene terephthalate (PET), polyethylene, polyurethane, oligocarbonatedimethacrylate (OCM-2) porous polymer, carbonate- and phthalate-containing dimethacrylates, and the like.

69. The method of claim 67, wherein said nanoparticle comprises a hydrogel.

70. The method of claim 69, wherein said hydrogel comprises a hydrogel formed from one or more materials selected from the group consisting of poly(N-isopropylacrylamide) (PNIPA), poly(N-isopropylacrylamide-co-1-vinylimidazole) (PNIPA-VI), poly(acrylamide) (PAAm), poly(acrylamide), poly(N,N-dimethylacrylamide), poly(N,N-diethylacrylamide), poly(1-vinylimidazole), poly(sodium acrylate), poly(sodium methacrylate), poly(2-hydroxyethylmethacrylate) (HEMA), poly(N,N-dimethylaminoethyl methacrylate) (DMAEMA), poly(N-[tris(hydroxymethyl)methyl]acrylamide), poly(1-(3-methacryloxy)propylsulfonic acid) (sodium salt), poly(allylamine), poly(N-acryloxysuccinimide), poly(N-vinylcaprolactam), poly(1-vinyl-2-pyrrolidone), poly(2-acrylamido-2-methyl-1-propanesulfonic acid) (sodium salt), poly((3-acrylamidopropyl) trimethylammonium chloride), and poly(diallyldimethylammonium chloride).

71. The method of claim 53, wherein said nanoparticle comprises a metal organic framework (MOF).

72. The method of claim 71, wherein said nanoparticle comprises a metal organic framework selected from the group consisting of zeolitic imidazolate frameworks (ZIFs), Universitetet i Oslo (University of Oslo) frameworks (UiOs), and (Materials of Institut Lavoisier frameworks (MILs).

73. The method of claim 72, wherein said nanoparticle comprises a metal organic framework selected from the group consisting of ZIF-8, ZIF-67, ZIF-90, Fe-BTC, HKUST-1, and MIL-53, MIL-89, MIL-88A, MIL-100, UiO-66, UiO-66-NH₂, MOF-801, MOF-804, Fe-NDC-M, MOF-1201, MOF-1203, and Fe-NDC-O MOFs.

74. The method of claim 73, wherein said nanoparticle comprises a MIL-88A MOF.

75. The method of claim 73, wherein said nanoparticle comprises a ZIF-8 MOF.

76. The method of claim 73, wherein said nanoparticle comprises a UiO-66 MOF, or a UiO-66-NH₂ MOF.

77. The method according to any one of claims 1-76, wherein said drug delivery vehicles have an average hydrodynamic diameter ranging from about 30 nm or about 50 nm up to about 300 nm, or from about 30 nm or about 50 nm up to about 200 nm, or from about 30 nm or about 50 nm up to about 170 nm, or from about 30 nm or about 50 nm up to about 150 nm, or from about 30 nm or about 50 nm up to about 100 nm, or from about 30 nm or about 50 nm up to about 80 nm, or from about 30 nm or about 50 nm up to about 70 nm, or from about 60 nm up to about 70 nm by DLS.

78. The method of claim 77, wherein said drug delivery vehicles have an average hydrodynamic diameter ranging from about 145 nm up to about 165 nm by DLS or from about 150 nm up to about 161 nm by DLS.

79. The method according to any one of claims 1-78, wherein said nanoparticle has an average pore size that ranges from about 1 to about 20 nm, or from about 1 to about 10 nm, or from about 1 to about 5 nm, or from about 1 to about 4 nm, or from about 1 to about 3 nm, or from about 2 to about 3 nm.

80. The method according to any one of claims 1-79, wherein said lipid bilayer comprises a phospholipid, and cholesterol (CHOL) and/or a cholesterol derivative.

81. The method of claim 80, wherein said lipid bilayer comprises a phospholipid and cholesterol (CHOL).

82. The method according to any one of claims 80-81, wherein said phospholipid comprises a saturated fatty acid with a C14-C20 carbon chain, and/or an unsaturated fatty acid with a C14-C20 carbon chain, and/or a natural lipid comprising a mixture of fatty acids with C12-C20 carbon chains.

83. The method of claim 82, wherein said phospholipid comprises one or more phospholipids selected from the group consisting of distearoylphosphatidylcholine (DSPC), phosphatidylcholine (DPPC), 1,2-dimyristoleoyl-sn-glycero-3-phosphocholine (DMPC), 1,2-dioleoyl-sn-glycero-3-phosphocholine (DOPC), 1,2-distearoyl-sn-glycero-3-phospho-rac-glycerol (DSPG), 1,2-dipalmitoyl-sn-glycero-3-phosphoglycerol (DPPG), 1,2-dieicosenoyl-sn-glycero-3-phosphocholine, and diactylphosphatidylcholine (DAPC), 1, 2-Distearoyl-sn-glycero-3-phosphoethanolamine (DSPE), and dipalmitoyl phosphatidylethanolamine.

84. The method of claim 82, wherein said phospholipid comprises a natural lipid selected from the group consisting of egg phosphatidylcholine (egg PC), and soy phosphatidylcholine (soy PC).

85. The method of claim 82, wherein said phospholipid comprises distearoylphosphatidylcholine (DSPC).

86. The method according to any one of claims 80-85, wherein said lipid bilayer comprises an mPEG phospholipid with a phospholipid C14-C18 carbon chain, and a PEG molecular weight ranging from about 350 Da to 5000 Da.

87. The method of claim 86, wherein said lipid bilayer comprises 1, 2-Distearoyl-sn-glycero-3-phosphoethanolamine-Poly(ethylene glycol) (DSP-PEG), or dipalmitoyl phosphatidylethanolamine grafted poly(ethylene glycol) (PE-PEG).

88. The method of claim 87, wherein said PE-PEG comprises DS PE-PEG_{2K}.

89. The method of claim 87, wherein said PE-PEG comprises DSPE-PEG_{5K}.

90. The method according to any one of claims 85-89, wherein said lipid bilayer comprises DPSC, cholesterol, and DSPE-PEG.

91. The method of claim 90, wherein the molar ratio of DPSC : cholesterol : PE-PEG ranges from 20-90% DSPC : 10%-50% Chol : 1%-10% DS PE-PEG.

92. The method of claim 91, wherein the molar ratio of DSPC : Chol :DS PE-PEG is about 3 : 2 : 0.15.

93. The method according to any one of claims 80-92, wherein said nanoparticles have a particle (e.g., MSNP):lipid ratio of 1:1.25 (w/w) or greater.

94. The method according to any one of claims 80-92, wherein said lipid bilayer comprises a cholesterol derivative selected from the group consisting of cholesterol hemisuccinate (CHEMS), lysine-based cholesterol (CHLYS), and PEGylated cholesterol (Chol-PEG).

95. The method of claim 94, wherein said cholesterol derivative is in place of said cholesterol.

96. The method according to any one of claims 94-95, wherein said lipid bilayer comprises CHEMS.

97. The method of claim 96, wherein said lipid bilayer comprises CHEMS ranging from about 5% (mol percent) up to about 30% total lipid.

98. The method of claim 97, wherein said lipid bilayer comprises about 10% or about 20% CHEMS or about 30% CHEMS or about 40% CHEMS.

99. The method according to any one of claims 1-98, wherein said one or more camptothecin analogs, and/or one or more autophagy inhibitors are loaded into said nanoparticle with a cargo trapping agent (e.g., protonating agent).

100. The method of claim 99, wherein said cargo trapping agent before reaction with the one or more camptothecin analogs, and/or one or more autophagy inhibitors is selected from the group consisting of triethylammonium sucrose octasulfate (TEA₈SOS), citric acid, (NH₄)₂SO₄, an ammonium salt, a trimethylammonium salt, and a triethylammonium salt.

101. The method according to any one of claims 1-100, wherein said drug delivery vehicle is conjugated to a moiety selected from the group consisting of a targeting moiety, a fusogenic peptide, and a transport peptide.

102. The method of claim 101, wherein said drug delivery vehicle is conjugated to a peptide that binds a receptor on a cancer cell or tumor blood vessel.

103. The method of claim 102, wherein said drug delivery vehicle is conjugated to an iRGD peptide.

104. The method of claim 102, wherein said drug delivery vehicle is conjugated to a targeting ligand shown in Table 4.

105. The method according to any one of claims 101-104, wherein said drug delivery vehicle is conjugated to transferrin, and/or ApoE, and/or folate.

106. The method according to any one of claims 101-105, wherein said drug delivery vehicle is conjugated to a targeting moiety that comprises an antibody that binds to a cancer marker.

107. The method of claim 106, wherein said drug delivery vehicle is conjugated to a targeting moiety that comprises an antibody that binds a cancer marker shown in Table 3.

108. The method according to any one of claims 106-107, wherein said antibody is selected from the group consisting of an intact immunoglobulin, an F(ab)′₂, a Fab, a single chain antibody, a diabody, an affibody, a unibody, and a nanobody.

109. The method according to any one of claims 1-108, wherein said drug delivery vehicles in suspension are stable for at least 1 month, or at least 2 months, or at least 3 months, or at least 4 months, or at least 5 months, or at least 6 months when stored at 4° C.

110. The method according to any one of claims 1-109, wherein said drug delivery vehicles form a stable suspension on rehydration after lyophilization.

111. The method according to any one of claims 1-110, wherein said methods, show reduced drug toxicity as compared to free one or more camptothecin analogs alone or in combination with said one or more autophagy inhibitors.

112. The method according to any one of claims 1-111, wherein said drug delivery vehicles have colloidal stability in physiological fluids with pH 7.4 and remains monodisperse to allow systemic biodistribution and are capable of entering a disease site by vascular leakage (EPR effect) or transcytosis.

113. The method drug carrier according to any one of claims 1-112, wherein said drug delivery vehicles are colloidally stable.

114. The method according to any one of claims 1-113, wherein said method comprises a component of a primary therapy in a chemotherapeutic regimen.

115. The method according to any one of claims 1-113, wherein said method comprises an adjunct therapy in a treatment regime that additionally comprises chemotherapy using another chemotherapeutic agent, and/or surgical resection of a tumor mass, and/or radiotherapy.

116. The method according to any one of claims 1-115, wherein said cancer comprises a solid tumor.

117. The method of claim 116, wherein said cancer comprises a cancer selected from the group consisting of pancreatic cancer, gastric cancer, hepatocellular carcinoma, head and neck squamous cell carcinoma, urothelial carcinoma, cervical cancer, non-small cell lung cancer, and broadly for non-respectable solid tumors with high microsatellite instability (MSI-H) or DNA mismatch repair deficiency.

118. The method according to any one of claims 114-117, wherein said cancer comprises pancreatic cancer.

119. The method according to any one of claims 114-117, wherein said cancer comprises colorectal cancer.

120. The method according to any one of claims 114-117, wherein said cancer comprises lung cancer.

121. The method according to any one of claims 114-117, wherein said cancer is a cancer selected from the group consisting of breast cancer, lung cancer, melanoma, pancreas cancer, liver cancer, acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm’s tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm’s tumor.

122. The method according to any one of claims 1-121, wherein administration of said one or more checkpoint inhibitor(s), and/or said one or more camptothecin analogs, and/or said one or more autophagy inhibitors is via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

123. The method according to any one of claims 1-121, wherein administration of said one or more checkpoint inhibitor(s), and/or said one or more camptothecin analogs, and/or said one or more autophagy inhibitors comprises systemic administration via injection or cannula.

124. The method according to any one of claims 1-121, wherein administration of said one or more checkpoint inhibitor(s), and/or said one or more camptothecin analogs, and/or said one or more autophagy inhibitors comprises administration to an intra-tumoral or peri-tumoral site.

125. The method according to any one of claims 1-124, wherein said mammal is a human.

126. The method according to any one of claims 1-124, wherein said mammal is a non-human mammal.

127. A pharmaceutical formulation comprising:
nanoparticle drug carrier according to any one of claims 1-113;
a checkpoint inhibitor; and
a pharmaceutically acceptable carrier.

128. The pharmaceutical formulation of claim 127, wherein said formulation is an emulsion, dispersion, or suspension.

129. The pharmaceutical formulation of claim 128, wherein said suspension, emulsion, or dispersion is stable for at least 1 month, or at least 2 months, or at least 3 months, or at least 4 months, or at least 5 months, or at least 6 months when stored at 4° C.

130. The pharmaceutical formulation according to any one of claims 127-129, wherein the nanovesicle drug carriers, and/or the a nanoparticle drug carriers, and/or the a nanomaterial carriers in said formulation show a substantially unimodal size distribution; and/or show a PDI less than about 0.2, or less than about 0.1.

131. The pharmaceutical formulation according to any one of claims 127-130, wherein said formulation is formulated for administration via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

132. The pharmaceutical formulation according to any one of claims 127-130, wherein said formulation is a sterile injectable.

133. The pharmaceutical formulation according to any one of claims 127-132, wherein said formulation is a unit dosage formulation.
